SEQUENCE LISTING (11 sequences)

SEQ ID NO: 1 - protein, 26 aa, Apis mellifera
  MISC_FEATURE (26)..(26): C terminus CONH2
GIGAVLKVLTTGLPALISWIKRKRQQ

SEQ ID NO: 2 - protein, 26 aa, Artificial Sequence
  C terminus COO-
  MISC_FEATURE (26)..(26): Modified C terminus
GIGAVLKVLTTGLPALISWIKRKRQQ

SEQ ID NO: 3 - protein, 26 aa, Artificial Sequence
  Melittin variant Mel-P1
GIGAVLKVLATGLPALISWIKRKRQQ

SEQ ID NO: 4 - protein, 26 aa, Artificial Sequence
  Melittin variant Mel-P2
GIGAVLKVLTTGLPALISWIKRARQQ

SEQ ID NO: 5 - protein, 26 aa, Artificial Sequence
  Melittin variant Mel-P3
GIGAVLKVLATGLPALISWIKRARQQ

SEQ ID NO: 6 - protein, 26 aa, Artificial Sequence
  Melittin variant Mel-P4
GIGAVLKVLATGLPALISWIQAAQQL

SEQ ID NO: 7 - protein, 26 aa, Artificial Sequence
  Melittin variant Mel-P5
GIGAVLKVLATGLPALISWIKAAQQL

SEQ ID NO: 8 - protein, 26 aa, Artificial Sequence
  Melittin variant Mel-P6
GIGAVLKVLATGLPALISWIKRAQQL

SEQ ID NO: 9 - protein, 26 aa, Artificial Sequence
  Melittin variant Mel-P7
GIGAVLKVLTTGLPALISWIKRAQQQ

SEQ ID NO: 10 - protein, 26 aa, Artificial Sequence
  Melittin variant Mel-P8
GIGAVLKGLATGLPALISWIQRAQQQ

SEQ ID NO: 11 - protein, 26 aa, Artificial Sequence
  Melittin variant Mel-P9
GIGAVLKGLTTGLPALISWIKAAQQL

(19) United States
(12) Patent Application Publication
(10) Pub. No.: US 2024/0009269 A1
(43) Pub. Date: Jan. 11, 2024

(54) MEMBRANE-ACTIVE PEPTIDES AND METHODS FOR REVERSIBLE BLOOD- BRAIN BARRIER OPENING

(71) Applicant: THE JOHNS HOPKINS UNIVERSITY, Baltimore, MD (US)

(72) Inventors: Peter Charles SEARSON, Baltimore, MD (US); Kalina HRISTOVA, Baltimore, MD (US); Raleigh LINVILLE, Baltimore, MD (US); Alexander KOMIN, Baltimore, MD (US); Piotr WALCZAK, Baltimore, MD (US)

(73) Assignee: THE JOHNS HOPKINS UNIVERSITY, Baltimore, MD (US)

(21) Appl. No.: 18/253,084

(22) PCT Filed: Nov. 18, 2021
(86) PCT No.: PCT/US2021/059938

§ 371 Date: May 16, 2023

Related U.S. Application Data
(60) Provisional application No. 63/116,381, filed on Nov. 20, 2020.

(51) Int. Cl. A61K 38/17; A61K 9/00; A61M 31/00

(52) U.S. Cl. CPC A61K 38/1767; A61K 9/0019; A61M 31/005; A61M 2210/0693; G01R 33/5601

(57) ABSTRACT

The present disclosure is directed to a composition or combination comprising at least one membrane-active peptide, such as melittin, and at least one therapeutic and/or diagnostic agent. Methods of using the membrane-active peptides of the disclosure to open a blood-brain barrier and to deliver a therapeutic and/or a diagnostic agent to a central nervous system (CNS) of a subject in need thereof are also provided. When administered as an intra-arterial injection into the cerebrovasculature, membrane-active peptides, such as melittin, support reversible blood-brain barrier opening without neurological damage

### CROSS REFERENCE TO RELATED APPLICATIONS

This application claims priority to U.S. Patent Application No. 63/116,381, filed on Nov. 20, 2020, the entire disclosure of which is hereby incorporated by reference in its entireties.

### GOVERNMENT INTEREST

This invention was made with government support under grants HDTRA1-15-0046 awarded by the Defense Threat Reduction Agency. The government has certain rights in the invention.

### SEQUENCE LISTING

The instant application contains a Sequence Listing which has been submitted electronically in ASCII format and is hereby incorporated by reference in its entirety. Said ASCII copy, created on 18 Nov. 2021, is named 0184_105_ST25.txt and is 4,141 bytes in size.

### FIELD

The present disclosure is directed to peptides useful for opening the blood-brain barrier and the use of these peptides for administering therapeutic and diagnostic agents into the brain.

### BACKGROUND

The blood-brain barrier remains a major roadblock for the delivery of therapeutics or diagnostic agents to the brain. While the ability to overcome or circumvent the blood-brain barrier has the potential to enable the development of drug, gene, and cell therapies for currently untreatable diseases, drug therapies to date have been largely limited to a relatively small number of lipophilic molecules that can passively diffuse across endothelial cell membranes.

In the 1990s, inhibition of multi-spectrum efflux transporters was explored as a strategy for increasing the number of small molecules that could enter the brain at clinically relevant doses, but this approach failed due to adverse side effects. Other strategies included hijacking receptor-mediated transport systems, such as the transferrin receptor, but these too have had limited success. For example, modulating transcellular permeability by efflux pump inhibition resulted in only a 2-3 fold increase in efflux substrate permeability in vivo and in vitro. Accordingly, such techniques to modulate blood-brain barrier permeability have resulted in only modest increases in therapeutic penetration.

In contrast to approaches that involve transcellular transport, blood-brain barrier opening has been achieved using reversible mechanical or chemical disruption of cell junctions to enable transient paracellular transport between endothelial cells. These approaches have the advantage of allowing entry of any therapeutic or diagnostic agent into the brain over a limited period of time, followed by complete recovery.

The most well-known method for mechanically-induced blood-brain barrier opening, magnetic resonance-guided focused ultrasound (FUS), exploits distortion of microbubbles injected into circulation to disrupt cell junctions. FUS enable precise spatial control of an opening, which can be advantageous compared to other methods, such as catheterization of arteries with distinct perfusion territories. However, FUS confers risk for off-target effects and is highly inefficient since only a small fraction of therapeutics enter the brain (as low as 0.009% of injected dose) due to systemic drug administration.

A well-known chemical method for blood-brain barrier opening is the intra-arterial injection of hyperosmotic agents, such as mannitol, which has been used in both preclinical models and clinical studies for several decades. Intra-arterial injection of mannitol results in endothelial cell shrinkage, which can induce local transient disruption of endothelial junctions. This approach has been used to improve delivery of chemotherapeutics, stem cells and viral vectors into the brain. However, widespread clinical use has been limited due to the lack of reproducibility. Further, mannitol only induces blood-brain barrier opening close to its solubility limit and, accordingly, generally requires complete displacement of blood volume.

Consequently, there remains a need for potent and safe compositions as well as methods, which may be reliably used to open the blood-brain barrier. The ability to provide compositions that may be modified to obtain customizable pharmacological properties is also desired.

### BRIEF DESCRIPTION OF THE DRAWINGS

The application file contains at least one drawing executed in color. Copies of this patent application publication with color drawings will be provided by the Office upon request and payment of the necessary fee.

Embodiments of the present disclosure will become more fully understood from the detailed description and the accompanying drawings, wherein:

FIGS. 1A-1D: show the development of membrane-active peptide-induced blood-brain barrier opening (mapBBBO) as described in the Examples. FIG. 1A shows a 3D model of melittin (RSCB Protein Data Bank (doi: 10.2210/pdb6dst/pdb)). FIG. 1B shows cytotoxicity assays, which are used to determine doses of melittin that minimally compromise brain microvascular endothelial cell and neuronal viability. FIG. 1C shows a tissue engineered human microvessel model, which is used to determine dose dependence and mechanisms of mapBBBO. FIG. 1D shows the intra-arterial injection of a MAP into a brain.

FIGS. 2A-2D: show melittin-induced cytotoxicity on derived human brain microvascular endothelial cells (dhBMECs) and human cortical neurons (HCNs) as described in the Examples. FIG. 2A shows a time course of cell viability for dhBMECs and HCNs incubated with 5 μM melittin. FIG. 2B shows viability half-life for dhBMECs and HCNs (mean±Standard Error of the Mean (SEM). FIGS. 2C and 2D show phase diagrams for dhBMECs and HCNs, respectively (98% cell viability).

FIGS. 3A-3D: show melittin-induced blood-brain barrier (BBB) opening within tissue-engineered dhBMECs microvessels as described in the Examples. FIG. 3A shows an experimental imaging protocol (microvessels were exposed to 1.5, 5 or 10 μM melittin for 2, 5 or 10 minutes). FIG. 3B shows 2-minute 1.5 μM (low dose), 5-minute 5 μM (intermediate dose), and 10-minute 10 μM (high dose) melittin time course fluorescence images. FIG. 3C shows heat maps for each melittin dose; the green bar corresponds to the Regions of Interest (ROI) displayed in FIG. 3A. FIG. 3D shows focal leaks visible from binarized images of 500 kDa dextran fluorescence at 30 minutes after initial melittin exposure for each dose (data collected across n=9 microvessels exposed to doses from 3-100 μM·minute).

FIGS. 4A-4D: show dose dependence of melittin-induced BBB opening within tissue-engineered dhBMECs microvessels (data collected across n=9 microvessels exposed to doses from 3-100 μM·minute; red lines represent lines of best fit) as described in the Examples. FIG. 4A shows the average permeability (Pavg) of 500 kDa dextran. FIG. 4B shows 500 kDa dextran cumulative focal leak density. FIG. 4C shows a 500 kDa dextran focal leak reversibility constant, defined as the time in which the last new focal leak emerges after initial melittin dosing. FIG. 4D shows focal leak density 24 hours later.

FIGS. 5A-5F: show cellular dynamics of melittin-induced BBB opening within tissue-engineered dhBMECs microvessels as described in the Examples. FIG. 5A shows a schematic illustration of barrier-disruptive and non-disruptive events observed during melittin exposure. FIG. 5B shows representative, phase contrast time course images of dhBMEC cell loss and cell division in microvessels. Events are manually traced using live-cell phase contrast imaging of dhBMEC monolayers; mitosis is visible as cell compression, alignment of chromosomes, and the formation of daughter cells; cell loss is visible as cell shrinkage and subsequent disappearance from the monolayer. FIGS. 5C-5E show cell loss, cell division, and net turnover rates for dhBMECs across melittin doses; cell loss displays a strong positive correlation to melittin dosage, while proliferation displays a weak negative correlation. FIG. 5F shows spatial correlation of cell loss events and focal leaks; percent of focal leaks with detected cell loss within 50 μm of the leak and within 4 minutes of the leak formation versus melittin dose; data collected across n=9 microvessels exposed to doses from 3-100 μM·minute. Red lines represent lines of best fit; black dotted lines represent mean rates for dhBMEC microvessels under homeostatic conditions.

FIGS. 6A-6F: show mechanisms of junctional disruption during melittin-induced blood-brain barrier opening (BBBO) within tissue-engineered dhBMECs microvessels as described in the Examples. FIG. 6A shows epifluorescence images of 500 kDa dextran-fluorescein at 0 minutes and 18 minutes (8 minutes after a ten-minute 10 μM melittin perfusion). FIG. 6B shows focal leaks visible as a paracellular pathway of fluorescence; 10 μm above or below this location the endothelium is intact. FIG. 6C shows time course of cumulative cell loss, cell gain, and focal leak formation for 50 μM·minute melittin dose; event counting was initiated 30 minutes before dosing. FIG. 6D shows cell swelling occurring at the site of focal leaks after the focal leak formation. FIG. 6E shows that tight junctions display stable localization under baseline conditions in dhBMECS with GFP-tagged ZO1. FIG. 6F shows that exposure to melittin induces cell contraction (white arrows) of discrete dhBMECs; confocal images were collected across n=3 BBB microvessels exposed to 50 μM·minute melittin.

FIGS. 7A-7G: show a profile of melittin-induced BBB opening in mice as described in the Examples. FIGS. 7A and 7B show a time course of T1-weighted gadolinium-enhanced magnetic resonance imaging and Evans Blue leakage following 3 μM·minute melittin-induced BBB opening; representative images are shown. FIG. 7C shows brain-region specific dynamics of melittin-induced BBBO; a significant difference between T1 intensities in the ipsilateral and contralateral hemispheres is observed in the hippocampus and thalamus/hypothalamus immediately after melittin dosing. FIG. 7D shows quantification of the area of BBBO using thresholding of T1 hyperintensities. n=4, 5 and 3 mice at 0 hours, 24 hours and 7 days, respectively for the 3 μM·min melittin dose. FIG. 7E shows T2-weighted gadolinium-enhanced imaging conducted prior to BBBO correlates perfusion and BBBO territories. FIGS. 7F-7G show that the fractional areas of brain perfusion and BBBO are well correlated (n=5); statistical significance was calculated by student's paired t-test and a one-way ANOVA with Tukey's post hoc test, * p<0.05; data presented as means±SEM.

FIGS. 8A-8E: show magnetic resonance imaging (MRI) and histological assessment 7 days following melittin-induced BBB opening as described in the Examples. FIG. 8A shows T2-weighted and susceptibility weighted imaging (SWI) images 7 days after BBBO showed no sign of brain damage or microhemorrhage, respectively. FIG. 8B shows histological staining with eriochrome cyanine, which revealed that there was no myelin demyelination 7 days after BBBO. FIG. 8C shows an immunohistochemical detection of neuroinflammation markers glial fibrillary acidic protein (GFAP) and ionized calcium binding adaptor molecule 1 (Iba1) in the contralateral and ipsilateral BBBO region. FIG. 8D shows an immunohistochemical detection of damage markers using a neuronal nuclear antigen (NeuN) and apoptotic marker (caspase-3) in the contralateral and ipsilateral BBBO region. FIG. 8E shows a comparison of normalized intensity of immunohistochemical images between the contralateral (control) anipsilateral BBBO region; representative images are shown. n=3 mice for each imaging technique or stain; statistical significance was calculated by a student's paired t-test; data presented as means±SEM.

FIGS. 9A-9D: show melittin-induced disruption of stem-cell derived brain microvascular endothelial cells (dhBMECs) in a transwell assay as described in the Examples. FIG. 9A shows dhBMECs monolayers expressing tight junction proteins zona occludens-1 (ZO-1), occludin, and claudin-5 under baseline conditions. FIG. 9B shows transendothelial electrical resistance (TEER) of dhBMEC monolayers before and after exposure to 5 μM melittin for 90 minutes on the day of the exposure (day 0) and the next day. FIG. 9C shows 500 kDa dextran permeability across dhBMECs, increased after treatment with 5 μM melittin for 90 minutes. FIG. 9D shows phase contrast image of dhBMECs cultured on glass slides and treated with 5 μM melittin for 90 minutes before and after washes with buffer; melittin treatment results in cell swelling and detachment; n=7 transwell replicates across three independent dhBMEC differentiations; statistical significance was calculated by a student's paired t-test,*** p<0.001, **p<0.01; data presented as means±SEM.

FIGS. 10A-10E: show cell viability of human cortical neurons (HCN) and dhBMECs after a continuous exposure to melittin as described in the Examples. FIG. 10A shows a schematic of a cytotoxicity assay; cells were incubated with Ethidium homodimer-1 (EthD1) dye, which entered cells with permeabilized membranes, and Hoechst dye, which stained nuclei of all cells; the cells were incubated with different melittin concentration together with the assay dyes and imaged using an epifluorescence microscope. FIG. 10B shows examples of fluorescence images and processed images to identify the number of EhtD1-positive and Hoechst-positive cells; these images are cropped portions of larger images that were used for the analysis in Cell Profiler [Carpenter, A. E., et al., Genome Biol., 2006. 7(10): p. R100]; cell viability was measured by the cell entry of membrane-impermeable Ethidium homodimer-1 dye. FIG. 10C shows the effect of exposure of different melittin concentrations on HCN viability. FIG. 10D shows the effect of exposure of different melittin concentrations on dhBMEC viability. FIG. 10E shows a direct comparison of HCN and dhBMEC viability for different melittin concentrations; data presented as means±SEM. n=5 experiments for dhBMECs and n=3 experiments for HCNs.

FIGS. 11A-11D: show fabrication and perfusion of tissue-engineered BBB microvessels as described in the Examples. FIG. 11A shows a fabrication schematic (from top to bottom): (i) rods are suspended within 7 mg mL-1 type I collagen hydrogels held within rectangular chambers patterned in polydimethylsiloxane (PDMS), (ii) rod removal leaves behind a 150 μm diameter channel that is stiffened using treatment with 20 mM genipin, (iii) dhBMECs are seeded into channel, and (iv) channels are perfused at approximately 2 dyne cm⁻². FIG. 11B shows an image of a flow system comprised on inlet and outlet medium reservoirs. FIG. 11C shows an image of a device undergoing live-cell imaging on microscope. FIG. 11D shows quantification of flow rates and shear stress over two days after seeding (day 0) (n=14 microvessels); data presented as means±SEM.

FIGS. 12A-12B show an analysis of BBB opening and recovery within tissue-engineered BBB microvessels as described in the Examples. FIG. 12A shows 6 mm by 0.6 mm images, which are sectioned into ten adjacent squares (ROIs #1-10); the normalized fluorescence intensity is plotted over time; average permeability is calculated using the line of best fit from t=0 to t=90 minutes, while instantaneous permeability is calculated using the line of best fit for 10-minute periods for each ROI. FIG. 12B shows raw 500 kDa dextran images binarized in ImageJ before manual counting of events to quantify focal leaks; red asterisks represent counted focal leaks.

FIGS. 13A-13D show melittin-induced BBB disruption within tissue-engineered BBB microvessels and an experimental imaging protocol as described in the Examples. FIG. 13A shows microvessels perfused with a control, i.e., dextran; control phase contrast and 500 kDa dextran fluorescence time course; a single representative ROI is shown at times 0, 30, 60 and 90 minutes. Microvessel structure remains stable and 500 kDa dextran displays negligible permeability. FIG. 13B shows microvessels perfused with dextran and 5 μM melittin for 90 minutes; melittin-exposed phase contrast and 500 kDa dextran fluorescence time course. Formation of defects in the endothelium (red arrows) and high 500 kDa dextran permeability are observed. FIG. 13C shows a comparison of 500 kDa dextran permeability in a 2D transwell assay and in BBB microvessels. FIG. 13D shows the leakage site density in control and melittin-treated microvessels. n=3 microvessels for each condition; statistical significance was calculated by one-way ANOVA with Tukey's post hoc test and a student's unpaired t-test, ***p<0.001; data presented as means±SEM.

FIG. 14 shows the reversibility of BBB opening 24 hours after melittin exposure. Thresholding of 500 kDa dextran fluorescence images collected 24 hours after melittin exposure are shown, as described in the Examples. Permeability was reversible 24 hours after the exposure, as 500 kDa dextran remains confined to lumen. Sustained disruption of microvessel barrier was visible as plumes of dye entering the hydrogel.

FIGS. 15A-15D show chemical structures of exemplary peptides of the disclosure as described in the Examples. FIG. 15A shows melittin-carboxy (SEQ ID NO: 2); FIG. 15B shows melittin-carboxyamide (SEQ ID NO: 1); FIG. 15C shows melittin variant Mel-P3 (SEQ ID NO: 5); FIG. 15D shows melittin variant Mel-P5 (SEQ ID NO: 7); FIG. 15E shows melittin “scramble” as described in the Examples.

FIGS. 16A-16H: show that melittin variants, but not scrambled melittin, induce reversible BBB opening as described in the Examples. FIGS. 16A-16D show a comparison of BBB opening metrics between scrambled melittin (Mel-Scramble) and melittin (red line) for different doses (25, 50 and 100 μM·minute). FIG. 16A shows an average of 500 kDa dextran permeability. FIG. 16B shows focal leak density. FIG. 16C shows focal leak reversibility constant, and FIG. 16D shows focal leak density one day after melittin exposure. FIGS. 16E-16H show a comparison of BBB opening metrics between melittin variants (symbols) and melittin (red line) for different doses (25, 50 and 100 μM·minutes). FIG. 16E shows average 500 kDa dextran permeability. FIG. 16F shows a focal leak density. FIG. 16G shows focal leak reversibility constant. FIG. 16H shows a focal leak density one day after melittin exposure; total of n=12 microvessels exposed to 25, 50 and 100 μM·min Mel-Scramble, Mel-CONH₂, MelP3, and MelP5; line of best fit for Mel-COO-data is shown in red as a comparison.

FIG. 17 shows immunofluorescence imaging of zona occludens-1 (ZO1) junctions after melittin exposure as described in the Examples. A large effect of melittin on ZO1 expression after 3 μm·minute (1.5 μM melittin, 2 minute incubation), 25 μM·minute (5 μM melittin, 5 minutes incubation) and 100 μM·minute (10 μM melittin, 10 minute incubation) was not observed; red arrows indicate that melittin treatment was associated with the detachment of cells from the monolayer. Significant cell detachment observed at a melittin dose of 450 μM·minute (5 μM melittin, 90 minute incubation). Representative images are shown.

FIG. 18 shows a time course analysis of dhBMEC behavior as described in the Examples. Plots of cumulative cell divisions, cell loss, and focal leaks for melittin doses are shown. The left y-axis represents cumulative % of all dhBMECs gained (green line) or lost (red line). The right y-axis represents cumulative number of focal leaks (black line). Cell loss was observed after focal leak formation. Each graph represents a single melittin dose (n=9 total).

FIGS. 19A-19C show an Evans Blue leakage assay to determine a minimum effective dose as described in the Examples. Representative images are shown of Evans Blue leakage across six mouse brain slices following 1 μM·minute (FIG. 19A), 3 μM·minute (FIG. 19B), and 5 μM·minute melittin exposure (FIG. 19C). At least n=3 mice were analyzed for each dose.

FIGS. 20A-20C show a comparison of melittin- and mannitol-induced BBB opening as described in the Examples. FIGS. 20A-20B show patterns of gadolinium distribution following 3 μM·minute melittin and 1.4 M·minute mannitol. Similar variability in BBB opening was observed. Imaging and administration protocols were standardized for both agents; each image represents a unique mouse. FIG. 20C shows a comparison of BBB opening area between approaches; n=5 for melittin and n=4 for mannitol; statistical significance was calculated by a student's unpaired t-test; data presented as means±SEM.

FIGS. 21A-21B show a perfusion territory of intra-arterial injections as described in the Examples. FIG. 21A shows dynamic gadolinium-T2 MRI images of mouse brain before the intra-arterial infusion of gadolinium, and 35 seconds and 75 seconds after the infusion at a rate of 150 μL minute⁻¹; mainly deep brain structures (hippocampus particularly) are perfused. FIG. 21B shows thresholding of gadolinium-T2 pre-melittin and gadolinium-T1 post-melittin to correlate perfusion and BBB opening territories.

FIGS. 22A-22D show an MRI and histological assessment 24 hours following melittin-induced BBB opening as described in the Examples. FIG. 22A shows that T2-weighted and SWI images 24 hours after BBB opening (BBBO) demontrated no sign of brain damage or microhemorrhage, respectively. FIG. 22B shows immunohistochemical detection of neuroinflammation markers glial fibrillary acidic protein (GFAP) and ionized calcium binding adaptor molecule 1 (Iba1) in the contralateral and ipsilateral BBBO region. FIG. 22C shows immunohistochemical detection of damage markers using a neuronal nuclear antigen (NeuN) and apoptotic marker (caspase-3) in the contralateral and ipsilateral BBBO region. FIG. 22D shows a comparison of normalized intensity of immunohistochemical images between the contralateral (control) and ipsilateral BBBO region. Representative images are shown; n=3 mice for each imaging technique or stain; statistical significance was calculated by a student's paired t-test; data presented as means±SEM.

FIG. 23 shows mechanisms of mapBBBO. At low doses (below 20 μM·minute in microvessels), endothelial cells are able to dynamically retain cell-cell contacts during cell division and cell loss, thereby maintaining barrier function and preventing focal leaks, which was not associated with disruption of cell-cell junctions or loss of viability. At intermediate doses (20 μM·min-50 μM·min in microvessels), the tensile forces resulting from cell contraction are sufficiently large to induce local disruption of weak cell junctions resulting in the formation of focal leaks. The density of contraction and disruption events is sufficiently low that they are isolated, and recovery of normal barrier function occurs rapidly. At large doses (>50 μM·minute in microvessels) the density of contraction events is sufficiently high that clusters of disrupted cell-cell junctions result in dramatic cell loss.

FIGS. 24A-24B show a retrospective comparison of mannitol and melittin-induced BBBO within tissue-engineered microvessels. FIG. 24A shows a focal leak density over a mannitol dose.

FIG. 24B shows a focal leak density over a melittin dose.

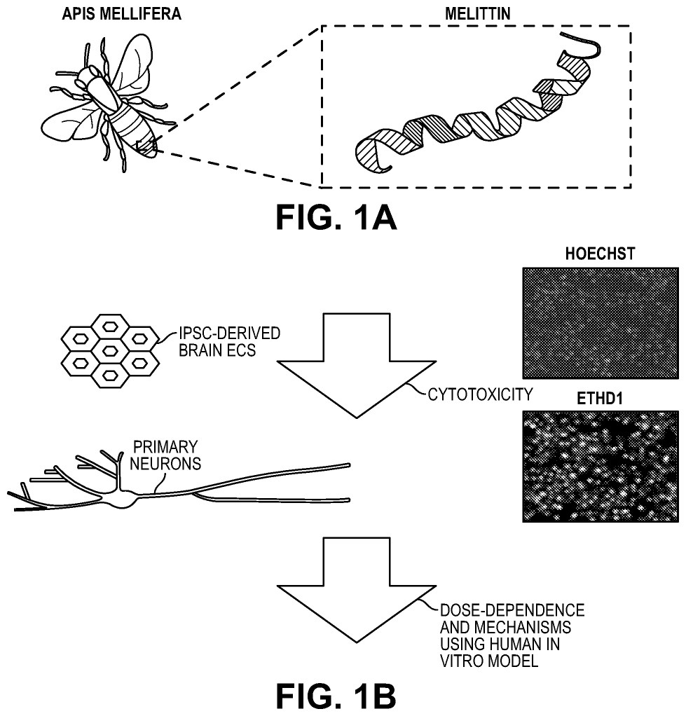

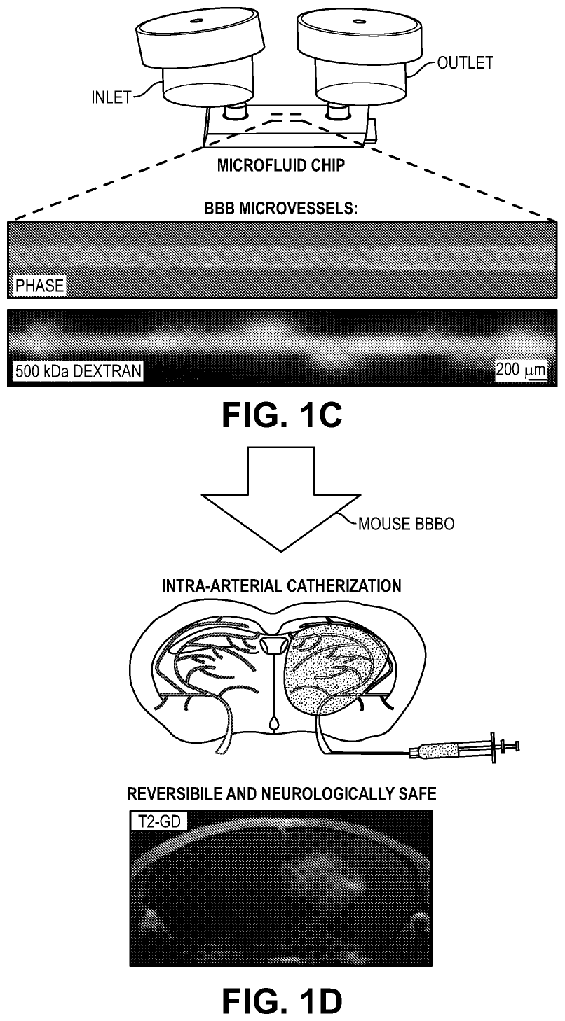

### SUMMARY

The present inventors have surprisingly recognized that membrane-active peptides may be reliably administered as herein described to open the blood-brain barrier. Blood-brain barrier opening using the membrane-active peptides of the disclosure have distinct advantages over existing approaches, including: (1) dramatic fold-increases in brain penetration for a wide range of molecular weights, (2) opening far from the solubility limit, (3) a large peptide design space to optimize opening, reversibility and therapeutic effect, and (4) lack of systemic and neuronal toxicity due to intra-arterial doses, which are diluted upon exiting the brain.

In one aspect, the present disclosure is directed to a composition or combination including: at least one membrane-active peptide comprising an amino acid sequence represented by Formula (I):

X₁X₂X₃X₄X₅X₆Z₁X₇X₈Z₂Z₃X₉X₁₀X₁₁X₁₂X₁₃X₁₄Z₄X₁₅X₁₆U₁U₂U₃U₄U₅U₆-B₁ (I),

wherein X₁-X₁₆ are each independently selected from a natural or a non-natural hydrophobic amino acid residue; wherein Z₁-Z₄ are each independently selected from a natural or a non-natural hydrophilic or hydrophobic amino acid residue, and wherein at least two of Z₁-Z₄ is independently selected from a natural or a non-natural hydrophilic amino acid residue; wherein U₁-U₆ are each independently selected from a natural or a non-natural hydrophobic or hydrophilic amino acid residue, and wherein at least three of U₁-U₆ is a natural or a non-natural hydrophilic amino acid residue, wherein B₁ is a terminus selected from COO— or CONH₂; at least one therapeutic and/or diagnostic agent; and wherein the composition or combination is optionally formulated for intraarterial injection.

In another aspect, the present disclosure is directed to a method of opening a blood-brain barrier in a subject including: administering to a subject at least one membrane-active peptide as herein described.

In yet another aspect, the present disclosure is directed to a method of delivering a therapeutic and/or a diagnostic agent to a central nervous system (CNS) of a subject in need thereof comprising: administering to the subject a composition or combination comprising at least one membrane-active peptide as herein described.

### DETAILED DESCRIPTION

The following description of the embodiments is merely exemplary in nature and is in no way intended to limit the disclosure, its application, or uses.

As used throughout, ranges are used as shorthand for describing each and every value that is within the range. Any value within the range can be selected as the terminus of the range. In addition, all references cited herein are hereby incorporated by reference in their entireties. In the event of a conflict in a definition in the present disclosure and that of a cited reference, the present disclosure controls.

Unless otherwise specified, all percentages and amounts expressed herein and elsewhere in the specification should be understood to refer to percentages by weight. The amounts given are based on the active weight of the material.

### Compositions and Combinations

Peptides

The present disclosure is directed to a composition or combination comprising at least one membrane-active peptide and at least one therapeutic and/or diagnostic agent as described herein. As also herein described, membrane-active peptides of the present disclosure are capable of opening a blood-brain barrier.

The membrane-active peptides of the present disclosure can be of any suitable length. Typically, the membrane-active peptides range from about 26 to 100 amino acids in length, such as about 26 to 50 amino acids in length, such as about 26 amino acids in length, with the provision that the activity of the peptide in blood-brain barrier opening is maintained. The membrane-active peptides also include those that are no more than 30 amino acids, no more than 29 amino acids, no more than 28 amino acids, no more than 27 amino acids, no more than 26 amino acids, or no more than 25 amino acids in length, no more than 24 amino acids, no more than 23 amino acids. Membrane-active peptide may be less than 100 amino acids, less than 50 amino acids, less than 30 amino acids, or less than 25 amino acids, with the provision that the activity of the peptide in blood-brain barrier opening is maintained.

Typically, the present membrane-active peptides are 26 amino acids in length and include natural and/or non-natural amino acids. As used herein, “natural amino acids” refer to the naturally encoded 20 common amino acids (alanine, arginine, asparagine, aspartic acid, cysteine, glutamine, glutamic acid, glycine, histidine, isoleucine, leucine, lysine, methionine, phenylalanine, proline, serine, threonine, tryptophan, tyrosine, and valine) and pyrolysine and selenocysteine.

As used herein a “non-natural amino acid,” an “amino acid analog”, a “non-canonical amino acid,” a “modified amino acid,” an “unnatural amino acid,” and the like may all be used interchangeably, and are meant to include amino acid-like compounds that are similar in structure and/or overall shape to one or more of the twenty common amino acids found in naturally occurring proteins and peptides.

In some embodiments, the non-natural amino acids of the present disclosure are selected or designed to provide additional characteristics to the twenty natural amino acids. For example, the membrane-active peptides of the disclosure may optionally be modified to include one or more non-natural amino acids, which alter or enhance certain properties of the instant membrane-active peptides including stability, e.g., thermal, hydrolytic, oxidative, resistance to enzymatic degradation, and the like, facility of purification and processing, spectroscopic properties, chemical and/or photochemical properties, half-life and the like.

In some embodiments, the non-natural amino acids share backbone structures and/or side chain structures of one or more natural amino acids, but include a modification, such as substitution of an atom (such as N) for a related atom (such as S), addition of a group (e.g., methyl, or hydroxyl.) or atom (such as Cl or Br, etc.), substitution of a covalent bond (single bond for double bond, etc.) or combinations thereof.

Examples of suitable unnatural amino acids for use in the present membrane-active peptides include D-amino acids, homo-amino acids, β-homo-amino acids, N-methyl amino acids and α-methyl amino acids. D-amino acids, which are the mirror image of the naturally occurring L-isomers, may increase resistance against degradation enzymes. Homo-amino acids are unnatural amino acids, wherein a methylene (CH₂) group is added to the α-carbon leading to increased biological stability. β-homo-amino acids are analogs of standard amino acids in which the carbon skeleton has been lengthened by insertion of one carbon atom immediately after the acid group. These amino acids can increase the in vivo half-life of peptides. N-methyl amino acids are amino acids that carry a methyl group at the nitrogen instead of a proton, leading to enhanced enzymatic stability. α-methyl amino acids are another type of natural amino acid variant, in which the proton on the α-carbon atom in between the amino and carboxy group of a natural amino acid has been substituted by a methyl group. α-methyl amino acids may increase the proteolytic stability of a peptide.

In some embodiments, the non-natural amino acids of the present disclosure comprise natural amino acids substituted with an alkyl, aryl, acyl, azido, cyano, halo, hydrazine, hydrazide, hydroxyl, alkenyl, alkynyl, ether, thiol, sulfonyl, seleno, ester, thioacid, borate, boronate, phospho, phosphono, phosphine, heterocyclic, enone, imine, aldehyde, hydroxylamine, keto, an amino or any combination thereof. In some embodiments, the non-natural amino acid includes a photoactivatable cross-linker. In some embodiments, the non-natural amino acid is a spin-labeled amino acid; a fluorescent amino acid; a metal binding amino acid; a metal-containing amino acid; a radioactive amino acid; a biotin or biotin-analogue containing amino acid; a keto containing amino acid; an amino acid comprising polyethylene glycol or polyether; a heavy atom substituted amino acid; a chemically cleavable or photocleavable amino acid; an amino acid with an elongated side chain; an amino acid containing a toxic group; a sugar substituted amino acid; a carbon-linked sugar-containing amino acid; a redox-active amino acid; an α-hydroxy containing acid; an amino thio acid; a cyclic amino acid other than proline or histidine, an aromatic amino acid other than phenylalanine, tyrosine or tryptophan, and/or the like.

Other examples of non-natural amino acids that may be used in the membrane-active peptides of the present disclosure include hydroxyproline, norleucine, 3-nitrotyrosine, nitroarginine and naphtylalanine.

In typical embodiments, the membrane-active peptides of the present disclosure include at least one amphipathic region. As used herein, an “amphipathic region” refers to a peptide region that possesses both hydrophobic and hydrophilic elements or characteristics, for example, a peptide region possessing a hydrophilic surface and a hydrophobic surface. In typical embodiments, the amphipathic region will be associated with a particular secondary structure, such as a helical structure, typically an amphipathic α-helical structure.

In certain embodiments, the amphipathic region of a membrane-active peptides of the present disclosure includes one or more (e.g., 1, 2, 3, 4, 5, 6, 7, 8, 9, 10 or more) hydrophobic amino acid residues, such as aromatic hydrophobic amino acid residues. Examples of aromatic hydrophobic amino acid residues include tryptophan (W), phenylalanine (F) and tyrosine (Y) and non-natural analogs thereof. In certain embodiments, the amphipathic region of the present disclosure includes one or more (e.g., 1, 2, 3, 4, 5, 6, 7, 8, 9, 10 or more) non-aromatic hydrophobic amino acid residues selected from glycine (G), proline (P), alanine (A), valine (V), leucine (L), isoleucine (I), methionine (M), and cysteine (C), more typically glycine (G), proline (P), alanine (A), valine (V), leucine (L) and isoleucine (I) and non-natural analogs thereof. In certain embodiments, the membrane-active peptides of the present disclosure have an amphipathic region that includes a combination of aromatic and non-aromatic natural and/or non-natural hydrophobic amino acid residues.

In certain embodiments, the amphipathic region of a membrane-active peptide of the present disclosure includes one or more (e.g., 1, 2, 3, 4, 5, 6, 7, 8, 9, 10 or more) hydrophilic amino acid residues. Examples of suitable hydrophilic amino acid residues include one or more polar amino acid residues such as lysine (K), arginine (R), histidine (H), threonine (T), serine (S), alanine (A), aspartic acid (D), glutamic acid (E), asparagine (N), glutamine (Q), and non-natural analogs thereof. Typically the hydrophilic amino acid residues include lysine (K), arginine (R), histidine (H), threonine (T), serine (S) and non-natural analogs thereof. In some embodiments, the amphipathic region includes from 1 to 8 hydrophilic amino acid residues, such as 1 to 4 hydrophilic amino acid residues or non-natural analogs thereof.

Specific examples of amphipathic regions, which maybe included in a membrane-active peptides of the present disclosure include: KVLTTGLPALISWI (residues 7-20 of SEQ ID NO: 1), KVLATGLPALISWI (residues 7-20 of SEQ ID NO: 3), KGLATGLPALIISSWI (residues 7-20 of SEQ ID NO: 9) and KGLTTGLPALISSWI (residues 7-20 of SEQ ID NO: 10).

In some embodiments, the at least one membrane-active peptide of the present disclosure comprises an amino acid sequence represented by Formula (I):

X₁X₂X₃X₄X₅X₆Z₁X₇X₈Z₂Z₃X₉X₁₀X₁₁X₁₂X₁₃X₁₄Z₄X₁₅X₁₆U₁U₂U₃U₄U₅U₆-B₁ (I)

In some embodiments, X₁-X₁₆ are each independently selected from a natural or a non-natural hydrophobic amino acid residue, such as hydrophobic amino acids selected from tryptophan (W), phenylalanine (W), tyrosine (Y), glycine (G), proline (P), alanine (A), valine (V), leucine (L), isoleucine (I), methionine (M), and cysteine (C), more typically glycine (G), proline (P), alanine (A), valine (V), leucine (L) and isoleucine (I) and non-natural analogs thereof.

In some embodiments, at least one of X₇, X₈, X₉, X₁₀ and/or X₁₁ is proline or a non-natural analog thereof; more typically X₁₁ is proline or a non-natural analog thereof. In other embodiments, X₁-X₆, X₁₂-X₁₄, X₁₅, X₁₆ are not proline or a non-natural analog thereof. In some embodiments, none of X₁-X₆ are proline.

In some embodiments, X₁, X₃ and X₉ are glycine, alanine, typically glycine, or a non-natural analog thereof. In some embodiments, X₂, X₄, X₅, X₆, X₈, X₁₀, X₁₂, X₁₃, X₁₄, X₁₆ are each independently selected from the group consisting of isoleucine, valine, leucine, alanine, glycine, more typically isoleucine, valine, leucine, alanine and a non-natural analog thereof. In some embodiments, X₇ is selected from the group consisting of valine, leucine, isoleucine, alanine, glycine, such as valine, glycine and a non-natural analog thereof. In some embodiments, X₁₅ is selected from the group consisting of tryptophan, phenylalanine, tyrosine, and a non-natural analog thereof.

In some embodiments, Z₁-Z₄ are each independently selected from a natural or a non-natural hydrophobic amino acid residue, such as tryptophan (W), phenylalanine (W), tyrosine (Y), glycine (G), proline (P), alanine (A), valine (V), leucine (L), isoleucine (I), methionine (M), and cysteine (C), more typically glycine (G), proline (P), alanine (A), valine (V), leucine (L) and isoleucine (I) and non-natural analogs thereof; however, more typically, Z₁-Z₄ are each independently selected from a hydrophilic amino acid or non-natural analog thereof, such as lysine (K), arginine (R), histidine (H), threonine (T), serine (S), alanine (A), glutamine (Q), aspartic acid (D), glutamic acid (E), asparagine (N), even more typically as lysine (K), arginine (R), histidine (H), threonine (T), serine (S), glutamine (Q) or a non-natural analog thereof. Yet even more typically, at least two of Z₁-Z₄ are each independently a hydrophilic residue, such as lysine (K), arginine (R), histidine (H), threonine (T), serine (S), alanine (A), and glutamine (Q), more typically lysine (K), arginine (R), histidine (H), threonine (T), serine (S), glutamine (Q) or a non-natural analog thereof.

In some embodiments, Z₁ is a natural or non-natural basic amino acid residue selected from amino acids such as lysine (K), arginine (R), histidine (H) or a non-natural analog thereof, more typically lysine (K), arginine (R) or a non-natural analog thereof.

In some embodiments, Z₂ is selected from the group consisting of threonine, alanine and a non-natural analog thereof.

In some embodiments, Z₃ and Z₄ are each independently selected from the group consisting of threonine, serine and a non-natural analog thereof.

In some embodiments, U₁-U₆ are each independently selected from a natural or a non-natural hydrophobic amino acid, such as tryptophan (W), phenylalanine (F), tyrosine (Y), glycine (G), proline (P), alanine (A), valine (V), leucine (L), isoleucine (I), methionine (M) and cysteine (C), more typically alanine (A), valine (V) and non-natural analogs thereof. Typically, U₁ to U₆ are not proline or a non-natural analog thereof.

In some embodiments, U₁-U₆ are each independently selected from a hydrophilic amino acid or non-natural analog thereof, such as lysine (K), arginine (R), histidine (H), threonine (T), serine (S), alanine (A), glutamine (Q), aspartic acid (D), glutamic acid (E), asparagine (N), more typically, lysine (K), arginine (R), histidine (H), threonine (T), serine (S), and glutamine (Q), even more typically lysine (K), arginine (R) and glutamine (Q) or a non-natural analog thereof. In some embodiments, at least three of U₁-U₆ is independently selected from a natural or a non-natural hydrophilic amino acid, such as lysine (K), arginine (R), histidine (H), threonine (T), serine (S), alanine (A), glutamine (Q), aspartic acid (D), glutamic acid (E), asparagine (N), typically lysine (K), arginine (R), histidine (H), threonine (T), serine (S), glutamine (Q), more typically lysine (K), arginine (R), glutamine (Q) or non-natural analogs thereof.

In other embodiments, U₁-U₆ are each independently selected from basic amino acids, such as lysine (K), arginine (R) and histidine (H) or non-natural analogs thereof, more typically lysine (K), arginine (R) or non-natural analogs thereof.

In some embodiments, U₁, U₄ and U₅ are each independently selected from a natural or non-natural hydrophilic amino acid residue, such as (K), arginine (R), histidine (H), threonine (T), serine (S), alanine (A), glutamine (Q) aspartic acid (D), glutamic acid (E), asparagine (N), more typically lysine (K), arginine (R), histidine (H), threonine (T), serine (S), glutamine (Q), even more typically lysine (K), arginine (R) and glutamine (Q) or a non-natural analog thereof. In some embodiments, U₂, U₃ and U₆ are each independently selected from a natural or a non-natural hydrophilic or hydrophobic amino acid residue, such as tryptophan (W), phenylalanine (F), tyrosine (Y), glycine (G), proline (P), alanine (A), valine (V), leucine (L), isoleucine (I), methionine (M) and cysteine (C), more typically alanine (A), valine (V) and non-natural analogs thereof.

In some embodiments, Formula I does not contain proline or a non-natural analog thereof.

In other embodiments, at least one of Z₁, X₁-X₁₆ and/or U₁ to U₆ is proline or a non-natural analog thereof.

In some embodiments, at least one of Z₁, X₁, X₂, X₇, X₈, Z₂, Z₃, X₉, X₁₀, X₁₁, X₁₅ and/or X₁₆ is proline or a non-natural analog thereof, wherein X₃-X₆, X₁₂-X₁₄, Z₄ and U₁ to U₆ are not proline or a non-natural analog thereof.

In yet other embodiments, at least one of Z₁, X₇ , X₈, Z₂, Z₃, X₉, X₁₀ and/or X₁₁ is proline or a non-natural analog thereof, wherein X₁-X₆, X₁₂-X₁₄, X₁₅, X₁₆, Z₄ and U₁ to U₆ are not proline or a non-natural analog thereof.

B₁ represents a terminus of the membrane-active peptides of the present disclosure. In some embodiments, the B₁ terminus is selected from COO—. In other embodiments, the B₁ terminus is selected from CONH₂.

In some embodiments, the at least one membrane-active protein is represented by Formula II:

GIGAVLZ₁X₇LZ₂TGLPALISWIU₁U₂U₃U₄U₅U₆-B₁ (II)

In some embodiments, Z₁ is selected from the group consisting of lysine, arginine and a non-natural analog thereof, X₇ is selected from valine, glycine or a non-natural analog thereof, Z₂ is selected from threonine, alanine or a non-natural analog thereof; and U₁-U₆ are each independently selected from the group consisting of lysine (K), arginine (R), alanine (A), leucine (L), glutamine (Q) and a non-natural analog thereof.

Specific examples of portion U peptide sequences (U₁-U₆) include: KRKRQQ (e.g., residues 21-26 of SEQ ID NO: 1), KRARQQ (e.g., residues 21-26 of SEQ ID NO: 3), QAAQQL (residues 21-26 of SEQ ID NO: 6), KAAQQL (e.g., residues 21-26 of SEQ ID NO: 7), KRAQQL (e.g., residues 21-26 of SEQ ID NO: 8), KRAQQQ (e.g., residues 21-26 of SEQ ID NO: 9), QRAQQQ (e.g., residues 21-26 of SEQ ID NO: 10), and KAAQQL (residues 21-26 of SEQ ID NO: 11).

In some embodiments, a membrane-active peptide of the present disclosure is melittin (also referred to herein as SEQ ID NO: 1, melittin, melittin-CONH₂ or melittin caboxyamide), which is the main component of European honey bee venom (Apis mellifera). In some embodiments, the at least one membrane-active peptide of the present disclosure is a modified melittin, wherein the carboxy terminus is COO—, instead of the CONH₂ found in melittin (also referred to herein as SEQ ID NO: 2, melittin-COO— or melittin carboxyamide). In some embodiments, the free carboxy C-terminus maximizes water solubility as described in the examples.

In some embodiments, a membrane-active peptide of the present disclosure has at least 75% identity to SEQ ID NO: 1 and is able to open the blood-brain barrier. As used herein “Percent (%) amino acid sequence identity” refers to the percentage of amino acid residues in a candidate sequence that are identical with the amino acid residues in the reference polypeptide sequence, such as a membrane-active peptide, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for example, using publicly available software such as BLAST or software available commercially, for example from DNASTAR. Two or more polypeptide sequences can be anywhere from 0-100% identical, or any integer value there between. In the context of the present disclosure, two polypeptides are “substantially identical” when at least 80% of the amino acid residues (such as at least about 85%, at least about 90%, at least about 92.5%, at least about 95%, at least about 98%, or at least about 99%) are identical.

In certain embodiments, a membrane-active peptide of the disclosure has 80% identity to SEQ ID NO: 1, such as 85% identity, such as 90% identity, such as 95% identity, such as 99% identity to SEQ ID NO: 1 and is capable of opening the blood-brain barrier. Examples of variants, which retain an ability to open the blood-brain barrier include those set forth as SEQ ID NOS: 3-11, which are also shown in the table below.

Table 1. Residues changed from SEQ ID NO: 1 (parent melittin sequence) in variants capable of opening the blood-brain barrier.

In certain embodiments, the at least one membrane-active peptide are selected from one or more of SEQ ID NOS: 1-11.

In some embodiments, the N-terminus of a membrane-active peptide as described herein is acetylated. In other embodiments, the N-terminus of a membrane-active peptide as described herein is conjugated to a fatty acid. In some embodiments, the N-terminus of a membrane-active peptide as described herein is conjugated to a sterol.

The membrane-active peptides of the disclosure can be prepared by various means, including recombinant expression, purification from cell culture and chemical synthesis. The membrane-active peptides of the disclosure can be provided in purified or substantially purified form i.e. substantially free from other polypeptides, e.g. free from naturally-occurring polypeptides, particularly from other host cell polypeptides, and are generally at least about 50% pure (by weight), and usually at least about 90% pure i.e. less than about 50%), and more typically less than about 10%) (e.g. 5%) of a composition is made up of other expressed polypeptides. Thus, in some embodiments, the membrane-active peptides of the disclosure are separated from the whole organism with which the molecule is expressed.

TABLE 1
Melittin and Melittin variants

| Peptide | Sequence* | Δ | + |
|---|---|---|---|
| SEQ ID NO: 1 | G I G A V L K V L T T G L P A L I S W I K R K R Q Q | 0 | 6 |
| (melittin) | | | |
| SEQ ID NO: 3 | G I G A V L K V L A T G L P A L I S W I K R K R Q Q | 1 | 6 |
| (Mel-P1) | | | |
| SEQ ID NO: 4 | G I G A V L K V L T T G L P A L I S W I K R A R Q Q | 1 | 5 |
| (Mel-P2) | | | |
| SEQ ID NO: 5 | G I G A V L K V L A T G L P A L I S W I K R A R Q Q | 2 | 5 |
| (Mel-P3) | | | |
| SEQ ID NO: 6 | G I G A V L K V L A T G L P A L I S W I Q A A Q Q L | 6 | 2 |
| (Mel-P4) | | | |
| SEQ ID NO: 7 | G I G A V L K V L A T G L P A L I S W I K A A Q Q L | 5 | 3 |
| (Mel-P5) | | | |
| SEQ ID NO: 8 | G I G A V L K V L A T G L P A L I S W I K R A Q Q L | 4 | 4 |
| (Mel-P6) | | | |
| SEQ ID NO: 9 | G I G A V L K V L T T G L P A L I S W I K R A Q Q Q | 2 | 4 |
| (Mel-P7) | | | |
| SEQ ID NO: 10 | G I G A V L K G L A T G L P A L I S W I Q R A Q Q Q | 4 | 3 |
| (Mel-P8) | | | |
| SEQ ID NO: 11 | G I G A V L K G L T T G L P A L I S W I K A A Q Q L | 4 | 3 |
| (Mel-P9) | | | |

*Bolded residues are basic amino acids; bolded underlined are changed from
SEQ ID NO: 1; A is the number of amino acid changes overall; “+” is the total
charge of the peptide.

Intraarterial Formulations

The compositions and/or combinations of the present disclosure may be formulated for any suitable mode of administration as described herein. For example, the compositions and/or combinations may be formulated for systemic administration. In other embodiments, the compositions and/or combinations may be formulated for local administration.

In some embodiments, the composition and/or combinations are formulated for parenteral, intradermal, subcutaneous, intravenous, or oral, administration. More typically, the therapeutic and/or diagnostic agent is formulated for systemic intravenous administration. Even more typically, the therapeutic and/or diagnostic agent is formulated for intra-arterial administration as described herein. In some embodiments, the present compositions may be formulated for intra-arterial administration to help to localize a composition and/or combination of the disclosure at specific sites as also herein described.

The term “combination” as used herein generally refers to a first composition comprising a membrane-active peptide of the present disclosure and another composition comprising a therapeutic and/or diagnostic agent of the present disclosure. The phrase “a composition or combination” refers to a composition containing a membrane-active peptide as well as a therapeutic and/or diagnostic agent or separate compositions, wherein the therapeutic and/or diagnostic agent is in one or more different composition(s) from that of the membrane-active peptide.

Typically, the compositions and/or combinations of the disclosure are formulated for intraarterial administration. Suitable formulations include solutions, suspensions, or emulsions (dispersions). Sterile liquids, such as water, saline, aqueous dextrose and related sugar solutions are typical liquid carriers that are included in the intraaterial formulations of the disclosure. Pharmaceutically acceptable preservatives, surfactants, viscosity enhancers, buffers, sodium chloride and water may also be included to form aqueous sterile solutions, suspensions and emulsions. An appropriate buffer system (e.g., sodium phosphate, sodium acetate, sodium citrate, or sodium borate) may also be used. Electrolytes, such as sodium, potassium, calcium, magnesium and/or chloride; an energy source, such as dextrose; and a buffer to maintain the pH of the solution may be included to obtain physiologically balanced solutions of the membrane-active peptides and optionally, the therapeutic and/or diagnostic agent of the disclosure. As used herein, the term “physiologically balanced solution” means a solution which is adapted to maintain the physical structure and function of tissues when introduced into the body. Various physiologically balanced solutions are known (e.g., Lactated Ringers Solution).

In some embodiments, one or more membrane-active peptides of the disclosure and optionally, a therapeutic or diagnostic agent of the disclosure, is formulated to avoid streaming. Streaming can occur during drug infusions when the currents within the arterial stream determine the distribution of a drug. Maldistribution can result in high concentrations of drug in some territories while bypassing others. In some embodiments, the present membrane-active peptides and optionally a therapeutic or diagnostic agent of the disclosure are formulated as a bolus to avoid streaming during infusion, enhance arterial concentration and/or to help to localize a membrane-active peptide and optionally, the therapeutic and/or diagnostic agents of the present disclosure at specific sites.

In some embodiments, the volume of the bolus is formulated to maximize capillary contact, i.e., displace blood in the arteries and the capillaries, but not the veins. Typically, excessive volume is avoided to mitigate rapid transit though the capillary networks, which decreases the time available for drug uptake. The concentration of the membrane-active peptides and optionally, the therapeutic or diagnostic agents of the present disclosure, may also be adjusted based on brain tissue uptake. Such determinations are within the skill of those in the art.

Diagnostic and Therapeutic Agents

In some embodiments, the compositions and/or combinations of the disclosure include a diagnostic and/or therapeutic agent. The present compositions, combinations and methods as described herein, may be used to increase drug delivery of any sized compound. Examples of suitable compounds include, but are not limited to an inorganic molecule, a peptide, a peptide mimetic, an antibody, a nucleic acid molecule or a combination thereof.

In some embodiments, the combinations and/or compositions include a diagnostic agent. Any suitable diagnostic agent may be used. Typical examples include contrast agents, radiopharmaceuticals, antisense radiopharmaceuticals, and peptide radiopharmaceuticals. Typical contrast agents include gadolinium and/or Feraheme or a combination thereof. Other suitable contrast agents include gadoterate, gadodiamide, gadobenate, gadopentetate, gadoteridol, gadoversetamide, gadoxetate, gadobutrol, gadoterate, gadodiamide, gadobenate, gadopentetate, gadoteridol, gadofosveset, gadoversetamide, gadoxetate and gadobutrol.

In some embodiments, the combinations or compositions include a therapeutic agent. The therapeutic agent of the composition and combination of the present disclosure may include any suitable therapeutic agent capable of treating a neurological disorder, including brain cancer. The therapeutic agent can include, for example, small molecule drugs, peptides, proteins, antibodies, RNAs (e.g., antisense therapy, RNAi therapy), DNAs (e.g., gene therapy, CRISPR therapy), anti-neoplastics, anti-infectives, anti-inflammatories (e.g., steroids, NSAIDs), seizure medications, psychotrophic medications, and medications for neurodegenerative diseases.

In some embodiments, the therapeutic agent can include any neurologically active agents, including those that act at synaptic and neuroeffector junction sites, e.g., cholinergic agonists, anticholinesterase agents, catecholamines and other sympathomimetic drugs, adrenergic receptor antagonists, an antimuscarinic drugs, and agents that act at the neuromuscular junction and autonomic ganglia.

Examples of suitable cholinergic agonists include choline chloride, acetylcholine chloride, methacholine chloride, carbachol chloride, bethanechol chloride, pilocarpine, muscarine, arecoline and the like. Suitable anticholinesterase agents include carbaril, physostigmine, neostigmine, edrophonium, pyridostigmine, demecarium, ambenonium, tetrahydroacridine and the like.

Suitable catecholamines and other sympathomimetic drugs include endogenous catecholamines, β-adrenergic agonists, α-adrenergic agonists and other miscellaneous adrenergic agonists. Within the subclass of endogenous catecholamines, suitable examples include epinephrine, norepinephrine, dopamine and the like. Suitable examples within the subclass of β-adrenergic agonists include isoproterenol, dobutamine, metaproterenol, terbutaline, albuterol, isoetharine, pirbuterol, bitolterol, ritodrine and the like. The subclass of α-adrenergic agonists includes methoxamine, phenylephrine, mephentermine, metaraminol, clonidine, guanfacine, guanabenz, methyldopa and the like. Other miscellaneous adrenergic agents include amphetamine, methamphetamine, methylphenidate, pemoline, ephedrine and ethylnorepinephrine and the like.

Adrenergic receptor antagonists include the subclasses of α-adrenergic receptor antagonists and β-adrenergic receptor antagonists. Suitable examples of α-adrenergic receptor antagonists include phenoxybenzamine and related haloalkylamines, phentolamine, tolazoline, prazosin and related drugs, ergot alkaloids and the like. Either selective or nonselective β-adrenergic receptor antagonists are suitable for use in the present disclosure, as are other miscellaneous β-adrenergic receptor antagonists.

Suitable antimuscarinic drugs include atropine, scopolamine, homatropine, belladonna, methscopolamine, methantheline, propantheline, ipratropium, cyclopentolate, tropicamide, pirenzepine and the like.

Therapeutic agents, which act at the neuromuscular junction and autonomic ganglia, are also contemplated. Suitable examples of such neurologically active agents include tubocurarine, alcuronium, β-erythroidine, pancuronium, gallamine, atracurium, decamethonium, succinylcholine, nicotine, labeline, tetramethylammonium, 1,1-dimethyl-4-phenylpiperazinium, hexamethonium, pentolinium, trimethaphan and mecamylamine, and the like.

The therapeutic agents of the disclosure also include those agents, which act on the central and peripheral nervous system. Such neurologically active agents can include nonpeptide neurotransmitters, peptide neurotransmitters and neurohormones, proteins associated with membranes of synaptic vessels, neuromodulators, neuromediators, sedative-hypnotics, antiepileptic therapeutic agents, therapeutic agents effective in the treatment of Parkinsonism and other movement disorders, opioid analgesics and antagonists and antipsychotic compounds.

Nonpeptide neurotransmitters include the subclasses of neutral amino acids, such as glycine and γ-aminobutyric acid and acidic amino acids, such as glutamate, aspartate, and NMDA receptor antagonist-MK801 (Dizocilpine Maleate). Other suitable nonpeptide neurotransmitters include acetylcholine and monoamines, such as dopamine, norepinephrine, 5-hydroxytryptamine, histamine, and epinephrine.

Neuroactive peptides, which are neurotransmitters and neurohormones, are also contemplated. These include subclasses of hypothalamic-releasing hormones, neurohypophyseal hormones, pituitary peptides, invertebrate peptides, gastrointestinal peptides and peptides found in the heart, e.g., atrial naturetic peptide.

Suitable examples of hypothalamic releasing hormones include thyrotropin-releasing hormones, gonadotropin-releasing hormone, somatostatins, corticotropin-releasing hormone and growth hormone-releasing hormone. Suitable examples of neurohypophyseal hormones include vasopressin, oxytocin, and neurophysins. Suitable examples of pituitary peptides include adrenocorticotropic hormone, beta-endorphin, alpha-melanocyte- stimulating hormone, prolactin, luteinizing hormone, growth hormone, and thyrotropin. Suitable invertebrate peptides include FMRF amide, hydra head activator, proctolin, small cardiac peptides, myomodulins, buccolins, egg-laying hormone and bag cell peptides. Suitable gastrointestinal peptides include vasoactive intestinal peptide, cholecystokinin, gastrin, neurotensin, methionine-enkephalin, leucine-enkephalin, insulin and insulin-like growth factors I and II, glucagon, peptide histidine isoleucineamide, bombesin, motilin and secretins.

Other suitable neuroactive peptides include angiotensin II, bradykinin, dynorphin, opiocortins, sleep peptide(s), calcitonin, CGRP (calcitonin gene-related peptide), neuropeptide Y, neuropeptide Yy, galanin, substance K (neurokinin), physalaemin, Kassinin, uperolein, eledoisin and atrial naturetic peptide.

Proteins associated with membranes of synaptic vesicles are also contemplated. These include the subclasses of calcium-binding proteins and other synaptic vesicle proteins. Examples of calcium-binding proteins include the cytoskeleton-associated proteins, such as caldesmon, annexins, calelectrin (mammalian), calelectrin (torpedo), calpactin I, calpactin complex, calpactin II, endonexin I, endonexin II, protein II, synexin I; and enzyme modulators. Synaptic vesicle proteins include inhibitors of mobilization (such as synapsin Ia,b and synapsin IIa,b), possible fusion proteins such as synaptophysin, and proteins of unknown function such as p29, VAMP-1,2 (synaptobrevin), VAT-1, rab 3A, and rab 3B.

Suitable therapeutic agents also include neuromodulators, such as CO₂ and ammonia, steroids and steroid hormones, adenosine and other purines, and prostaglandins; neuromediators, such as cyclic AMP, cyclic GMP and cyclic nucleotide-dependent protein phosphorylation reactions; and sedative-hypnotics, such as benzodiazepines and buspirone, barbiturates, and miscellaneous sedative-hypnotics.

Suitable therapeutic agents also include antiepileptic drugs, such as hydantoins including phenytoin, mephenytoin, and ethotoin; anticonvulsant barbiturates e.g., phenobarbital and mephobarbital; deoxybarbiturates, such as primidone; iminostilbenes, e.g., carbamazepine; succinimides, e.g., ethosuximide, methsuximide, and phensuximide; valproic acid; oxazolidinediones, e.g., trimethadione and paramethadione; benzodiazepines and other antiepileptic agents such as phenacemide, acetazolamide, and progabide.

Neurologically active agents that are effective in the treatment of Parkinsonism and other movement disorders are also contemplated. These agents include dopamine, levodopa, carbidopa, amantadine, baclofen, diazepam, dantrolene, dopaminergic agonists, such as apomorphine; ergolines, such as bromocriptine, pergolide and lisuride; and anticholinergic drugs, such as benztropine mesylate, trihexyphenidyl hydrochloride, procyclidine hydrochloride, biperiden hydrochloride, ethopropazine hydrochloride, and diphenhydramine hydrochloride.

The therapeutic agents of the disclosure also include opioid analgesics and antagonists. Suitable examples include endogenous opioid peptides, such as enkephalins, endorphins, and dynorphins; morphine and related opioids, such as levorphanol and congeners; meperidine and congeners, such as piperidine, phenylpiperidine, diphenoxylate, loperamide, and fentanyl; methadone and congeners, such as methadone and propoxyphene; pentazocine; nalbuphine; butorphanol; buprenorphine; meptazinol; opioid antagonists, such as naloxone hydrochloride; and centrally active antitussive agents, such as dextromethorphan.

Neurologically active agents that can be used to treat depression, anxiety or psychosis are also useful as therapeutic agents of the present disclosure. Suitable antipsychotic compounds include phenothiazines, thioxanthenes, dibenzodiazepines, butyrophenones, diphenylbutylpiperidines, indolones, and rauwolfia alkaloids. Mood alteration drugs, which may be used as therapeutic agents of the disclosure, include tricyclic antidepressants, including tertiary amines and secondary amines, atypical antidepressants, and monoamine oxidase inhibitors. Examples of suitable therapeutic agents useful in the treatment of anxiety include benzodiazepines.

Other suitable therapeutic agents include neuroactive proteins, such as human and chimeric mouse/human monoclonal antibodies, erythropoietin and G-CSF, orthoclone OKT3, interferon-gamma, interleukin-1 receptors, t-PA (tissue-type plasminogen activator), recombinant streptokinase, superoxide dismutase and tissue factor pathway inhibitor (TFPI).

Other neurologically active agent useful as therapeutic agents include neuroactive nonprotein drugs, such as neurotransmitter receptors and pharmacological targets in Alzheimer's disease; muscarinic agonists for the central nervous system; serotonic receptors and agents, thiazole-containing 5-hydroxytryptamine-3 receptor antagonists; acidic amino acids as probes of glutamate receptors and transporters; and L-2-(carboxycyclopropyl)glycines; and N-Methyl-D-aspartic acid receptor antagonists.

Therapeutic agents also include biotechnology drugs including those under development. Such drugs are exemplified in Tables 1 and 2 of U.S. Pat. No. 5,604,198, which is herein incorporated by reference in its entirety.

Suitable therapeutic agents also include cancer agents that may be administered through the blood-brain barrier. Such anti-cancer agents include 20-epi-1,25 dihydroxyvitamin D3, 4-ipomeanol, 5-ethynyluracil, 9-dihydrotaxol, abiraterone, acivicin, aclarubicin, acodazole hydrochloride, acronine, acylfulvene, adecypenol, adozelesin, aldesleukin, all-tk antagonists, altretamine, ambamustine, ambomycin, ametantrone acetate, amidox, amifostine, aminoglutethimide, aminolevulinic acid, amrubicin, amsacrine, anagrelide, anastrozole, andrographolide, angiogenesis inhibitors, antagonist D, antagonist G, antarelix, anthramycin, anti-dorsalizing morphogenetic protein-1, antiestrogen, antineoplaston, antisense oligonucleotides, aphidicolin glycinate, apoptosis gene modulators, apoptosis regulators, apurinic acid, ARA-CDP-DL-PTBA, arginine deaminase, asparaginase, asperlin, asulacrine, atamestane, atrimustine, axinastatin 1, axinastatin 2, axinastatin 3, azacitidine, azasetron, azatoxin, azatyrosine, azetepa, azotomycin, baccatin III derivatives, balanol, batimastat, benzochlorins, benzodepa, benzoylstaurosporine, beta lactam derivatives, beta-alethine, betaclamycin B, betulinic acid, BFGF inhibitor, bicalutamide, bisantrene, bisantrene hydrochloride, bisaziridinylspermine, bisnafide, bisnafide dimesylate, bistratene A, bizelesin, bleomycin, bleomycin sulfate, BRC/ABL antagonists, breflate, brequinar sodium, bropirimine, budotitane, busulfan, buthionine sulfoximine, cactinomycin, calcipotriol, calphostin C, calusterone, camptothecin derivatives, canarypox IL-2, capecitabine, caracemide, carbetimer, carboplatin, carboxamide-amino-triazole, carboxyamidotriazole, carest M3, carmustine, cam 700, cartilage derived inhibitor, carubicin hydrochloride, carzelesin, casein kinase inhibitors, castanospermine, cecropin B, cedefingol, cetrorelix, chlorambucil, chlorins, chloroquinoxaline sulfonamide, cicaprost, cirolemycin, cisplatin, cis-porphyrin, cladribine, clomifene analogs, clotrimazole, collismycin A, collismycin B, combretastatin A4, combretastatin analog, conagenin, crambescidin 816, crisnatol, crisnatol mesylate, cryptophycin 8, cryptophycin A derivatives, curacin A, cyclopentanthraquinones, cyclophosphamide, cycloplatam, cypemycin, cytarabine, cytarabine ocfosfate, cytolytic factor, cytostatin, dacarbazine, dacliximab, dactinomycin, daunorubicin hydrochloride, decitabine, dehydrodidemnin B, deslorelin, dexifosfamide, dexormaplatin, dexrazoxane, dexverapamil, dezaguanine, dezaguanine mesylate, diaziquone, didemnin B, didox, diethylnorspermine, dihydro-5-azacytidine, dioxamycin, diphenyl spiromustine, docetaxel, docosanol, dolasetron, doxifluridine, doxorubicin, doxorubicin hydrochloride, droloxifene, droloxifene citrate, dromostanolone propionate, dronabinol, duazomycin, duocarmycin SA, ebselen, ecomustine, edatrexate, edelfosine, edrecolomab, eflornithine, eflornithine hydrochloride, elemene, elsamitrucin, emitefur, enloplatin, enpromate, epipropidine, epirubicin, epirubicin hydrochloride, epristeride, erbulozole, erythrocyte gene therapy vector system, esorubicin hydrochloride, estramustine, estramustine analog, estramustine phosphate sodium, estrogen agonists, estrogen antagonists, etanidazole, etoposide, etoposide phosphate, etoprine, exemestane, fadrozole, fadrozole hydrochloride, fazarabine, fenretinide, filgrastim, finasteride, flavopiridol, flezelastine, floxuridine, fluasterone, fludarabine, fludarabine phosphate, fluorodaunorunicin hydrochloride, fluorouracil, flurocitabine, forfenimex, formestane, fosquidone, fostriecin, fostriecin sodium, fotemustine, gadolinium texaphyrin, gallium nitrate, galocitabine, ganirelix, gelatinase inhibitors, gemcitabine, gemcitabine hydrochloride, glutathione inhibitors, hepsulfam, heregulin, hexamethylene bisacetamide, hydroxyurea, hypericin, ibandronic acid, idarubicin, idarubicin hydrochloride, idoxifene, idramantone, ifosfamide, ilmofosine, ilomastat, imidazoacridones, imiquimod, immunostimulant peptides, insulin-like growth factor-1 receptor inhibitor, interferon agonists, interferon alpha-2A, interferon alpha-2B, interferon alpha-N1, interferon alpha-N3, interferon beta-IA, interferon gamma-IB, interferons, interleukins, iobenguane, iododoxorubicin, iproplatin, irinotecan, irinotecan hydrochloride, iroplact, irsogladine, isobengazole, isohomohalicondrin B, itasetron, jasplakinolide, kahalalide F, lamellarin-N triacetate, lanreotide, lanreotide acetate, leinamycin, lenograstim, lentinan sulfate, leptolstatin, letrozole, leukemia inhibiting factor, leukocyte alpha interferon, leuprolide acetate, leuprolide/estrogen/progesterone, leuprorelin, levamisole, liarozole, liarozole hydrochloride, linear polyamine analog, lipophilic disaccharide peptide, lipophilic platinum compounds, lissoclinamide 7, lobaplatin, lombricine, lometrexol, lometrexol sodium, lomustine, lonidamine, losoxantrone, losoxantrone hydrochloride, lovastatin, loxoribine, lurtotecan, lutetium texaphyrin, lysofylline, lytic peptides, maitansine, mannostatin A, marimastat, masoprocol, maspin, matrilysin inhibitors, matrix metalloproteinase inhibitors, maytansine, mechlorethamine hydrochloride, megestrol acetate, melengestrol acetate, melphalan, menogaril, merbarone, mercaptopurine, meterelin, methioninase, methotrexate, methotrexate sodium, metoclopramide, metoprine, meturedepa, microalgal protein kinase C inhibitors, MIF inhibitor, mifepristone, miltefosine, mirimostim, mismatched double stranded RNA, mitindomide, mitocarcin, mitocromin, mitogillin, mitoguazone, mitolactol, mitomalcin, mitomycin, mitomycin analogs, mitonafide, mitosper, mitotane, mitotoxin fibroblast growth factor-saporin, mitoxantrone, mitoxantrone hydrochloride, mofarotene, molgramostim, monoclonal antibody, human chorionic gonadotrophin, monophosphoryl lipid a/myobacterium cell wall SK, mopidamol, multiple drug resistance gene inhibitor, multiple tumor suppressor 1-based therapy, mustard anticancer agent, mycaperoxide B, mycobacterial cell wall extract, mycophenolic acid, myriaporone, n-acetyldinaline, nafarelin, nagrestip, naloxone/pentazocine, napavin, naphterpin, nartograstim, nedaplatin, nemorubicin, neridronic acid, neutral endopeptidase, nilutamide, nisamycin, nitric oxide modulators, nitroxide antioxidant, nitrullyn, nocodazole, nogalamycin, n-substituted benzamides, O6-benzylguanine, octreotide, okicenone, oligonucleotides, onapristone, ondansetron, oracin, oral cytokine inducer, ormaplatin, osaterone, oxaliplatin, oxaunomycin, oxisuran, paclitaxel, paclitaxel analogs, paclitaxel derivatives, palauamine, palmitoylrhizoxin, pamidronic acid, panaxytriol, panomifene, parabactin, pazelliptine, pegaspargase, peldesine, peliomycin, pentamustine, pentosan polysulfate sodium, pentostatin, pentrozole, peplomycin sulfate, perflubron, perfosfamide, perillyl alcohol, phenazinomycin, phenylacetate, phosphatase inhibitors, picibanil, pilocarpine hydrochloride, pipobroman, piposulfan, pirarubicin, piritrexim, piroxantrone hydrochloride, placetin A, placetin B, plasminogen activator inhibitor, platinum complex, platinum compounds, platinum-triamine complex, plicamycin, plomestane, porfimer sodium, porfiromycin, prednimustine, procarbazine hydrochloride, propyl bis-acridone, prostaglandin J2, prostatic carcinoma antiandrogen, proteasome inhibitors, protein A-based immune modulator, protein kinase C inhibitor, protein tyrosine phosphatase inhibitors, purine nucleoside phosphorylase inhibitors, puromycin, puromycin hydrochloride, purpurins, pyrazofurin, pyrazoloacridine, pyridoxylated hemoglobin polyoxyethylene conjugate, RAF antagonists, raltitrexed, ramosetron, RAS farnesyl protein transferase inhibitors, RAS inhibitors, RAS-GAP inhibitor, retelliptine demethylated, rhenium RE 186 etidronate, rhizoxin, riboprine, ribozymes, RH retinamide, RNAi, rogletimide, rohitukine, romurtide, roquinimex, rubiginone B1, ruboxyl, safingol, safingol hydrochloride, saintopin, sarcnu, sarcophytol A, sargramostim, SDI 1 mimetics, semustine, senescence derived inhibitor 1, sense oligonucleotides, signal transduction inhibitors, signal transduction modulators, simtrazene, single chain antigen binding protein, sizofiran, sobuzoxane, sodium borocaptate, sodium phenylacetate, solverol, somatomedin binding protein, sonermin, sparfosate sodium, sparfosic acid, sparsomycin, spicamycin D, spirogermanium hydrochloride, spiromustine, spiroplatin, splenopentin, spongistatin 1, squalamine, stem cell inhibitor, stem-cell division inhibitors, stipiamide, streptonigrin, streptozocin, stromelysin inhibitors, sulfinosine, sulofenur, superactive vasoactive intestinal peptide antagonist, suradista, suramin, swainsonine, synthetic glycosaminoglycans, talisomycin, tallimustine, tamoxifen methiodide, tauromustine, tazarotene, tecogalan sodium, tegafur, tellurapyrylium, telomerase inhibitors, teloxantrone hydrochloride, temoporfin, temozolomide, teniposide, teroxirone, testolactone, tetrachlorodecaoxide, tetrazomine, thaliblastine, thalidomide, thiamiprine, thiocoraline, thioguanine, thiotepa, thrombopoietin, thrombopoietin mimetic, thymalfasin, thymopoietin receptor agonist, thymotrinan, thyroid stimulating hormone, tiazofurin, tin ethyl etiopurpurin, tirapazamine, titanocene dichloride, topotecan hydrochloride, topsentin, toremifene, toremifene citrate, totipotent stem cell factor, translation inhibitors, trestolone acetate, tretinoin, triacetyluridine, triciribine, triciribine phosphate, trimetrexate, trimetrexate glucuronate, triptorelin, tropisetron, tubulozole hydrochloride, turosteride, tyrosine kinase inhibitors, tyrphostins, UBC inhibitors, ubenimex, uracil mustard, uredepa, urogenital sinus-derived growth inhibitory factor, urokinase receptor antagonists, vapreotide, variolin B, velaresol, veramine, verdins, verteporfin, vinblastine sulfate, vincristine sulfate, vindesine, vindesine sulfate, vinepidine sulfate, vinglycinate sulfate, vinleurosine sulfate, vinorelbine, vinorelbine tartrate, vinrosidine sulfate, vinxaltine, vinzolidine sulfate, vitaxin, vorozole, zanoterone, zeniplatin, zilascorb, zinostatin, zinostatin stimalamer, and zorubicin hydrochloride, as well as salts, homologs, analogs, derivatives, enantiomers and/or functionally equivalent compositions thereof.

Other suitable anti-cancer agents include Ributaxin, Herceptin, QUADRAMET®, Panorex, IDEC-Y2B8, BEC2, C225, Oncolym, Smart M195, Atragen, Ovarex, BEXXAR®, LDP-03, ior t6, MDX-210, MDX-11, MDX-22, OV103, 3622W94, anti-VEGF, Zenapax, MDX-220, MDX-447, MELIMMUNE-2, MELIMMUNE-1, Ceacide, Pretarget, NovoMAb-G2, TNT, Gliomab-H, GNI-250, EMD-72000, LymphoCide, CMA 676, Monopharm-C, 4B5, ior egf.r3, ior c5, BABS, anti-FLK-2, MDX-260, ANA Ab, Smart 1D10 Ab, SMART ABL 364 Ab and ImmuRAIT-CEA.

Therapeutic Antibodies

Suitable therapeutic agents also include therapeutic antibodies. The term “antibody” as used herein refers to immunoglobulin molecules and immunologically active portions of immunoglobulin (Ig) molecules, i.e., molecules that contain an antigen-binding site that specifically binds (immunoreacts with) an antigen, comprising at least one, and typically two, heavy (H) chain variable regions (abbreviated herein as VH), and at least one and typically two light (L) chain variable regions (abbreviated herein as VL). Such antibodies include polyclonal, monoclonal, chimeric, single chain, Fab, Fab′ and F(ab′)2 fragments. The VH and VL regions can be further subdivided into regions of hypervariability, i.e. “complementarity determining regions” (“CDR”), interspersed with regions that are more conserved, i.e. “framework regions” (FR). Each VH and VL is composed of three CDR's and four FRs, arranged from amino-terminus to carboxy-terminus in the following order: FR1, CDR1, FR2, CDR2, FR3, CDR3, FR4. In general, antibody molecules obtained from humans relate to any of the classes IgG, IgM, IgA, IgE and IgD, which differ from one another by the nature of the heavy chain present in the molecule. Certain classes have subclasses as well, such as IgG1, IgG2, and others. Furthermore, in humans, the light chain may be a kappa chain or a lambda chain. Reference herein to antibodies includes a reference to all such classes, subclasses and types of human antibody species.

Antibodies can be prepared using intact polypeptide as the immunizing agent or fragments thereof. A typical antigenic polypeptide fragment is typically 15-100 contiguous amino acids of a protein antigen of interest. In some embodiments, the peptide is located in a non-transmembrane domain of the polypeptide, e.g., in an extracellular or intracellular domain. An exemplary antibody or antibody fragment binds to an epitope that is accessible from the extracellular milieu and that alters the functionality of the protein. In certain embodiments, the present disclosure comprises therapeutic agents, which are antibodies that recognize and are specific for one or more epitopes of a protein antigen of interest.

The preparation of monoclonal antibodies are well known and can be obtained, for example, by injecting mice or rabbits with a composition comprising an antigen, verifying the presence of antibody production by removing a serum sample, removing the spleen to obtain B lymphocytes, fusing the lymphocytes with myeloma cells to produce hybridomas, cloning the hybridomas, selecting positive clones that produce antibodies to the antigen, and isolating the antibodies from the hybridoma cultures. Monoclonal antibodies can be isolated and purified from hybridoma cultures by techniques well known in the art.

In other embodiments, the antibody can be recombinantly produced, e.g., produced by phage display or by combinatorial methods. Phage display and combinatorial methods can be used to isolate recombinant antibodies that bind to a target disease peptide in the brain or fragments thereof (as described in, e.g., Ladner et al. U.S. Pat. No. 5,223,409, which is incorporated herein by reference in its entirety. Human monoclonal antibodies, humanized antibodies, Fab fragments, single chain antibodies and bispecific antibodies are contemplated.

Heteroconjugate antibodies are also contemplated as useful therapeutic agents. Heteroconjugate antibodies are composed of two covalently joined antibodies. Such antibodies have, for example, been proposed to target immune system cells to unwanted cells (see e.g., U.S. Pat. No. 4,676,980, which is herein incorporated by reference in its entirety) and for treatment of HIV infection (WO 91/00360, WO 92/200373, EP 03089, which are each herein incorporated by reference in its entirety). It is contemplated that the antibodies can be prepared in vitro using known methods in synthetic protein chemistry, including those involving crosslinking agents. For example, immunotoxins can be constructed using a disulfide exchange reaction or by forming a thioether bond. Examples of suitable reagents for this purpose include iminothiolate and methyl-4-mercaptobutyrimidate and those disclosed, for example, in U.S. Pat. No. 4,676,980, which is herein incorporated by reference in its entirety.

Suitable therapeutic agents also include immunoconjugates comprising an antibody conjugated to a chemical agent, or a radioactive isotope (i.e., a radioconjugate) for administration to the brain. Conjugates of an antibody and cytotoxic agent are prepared according to well known methods using a variety of bifunctional protein-coupling agents such as N-succinimidyl-3-(2-pyridyldithiol) propionate (SPDP), iminothiolane (IT), bifunctional derivatives of imidoesters (such as dimethyl adipimidate HCL), active esters (such as disuccinimidyl suberate), aldehydes (such as glutareldehyde), bis-azido compounds (such as bis (p-azidobenzoyl) hexanediamine), bis-diazonium derivatives (such as bis-(p-diazoniumbenzoyl)-ethylenediamine), diisocyanates (such as tolyene 2,6-diisocyanate), and bis-active fluorine compounds (such as 1,5-difluoro-2,4-dinitrobenzene). For example, a ricin immunotoxin can be prepared as described in Vitetta et al., Science, 238: 1098 (1987), which is herein incorporated by reference in its entirety. Carbon-14-labeled 1-isothiocyanatobenzyl-3-methyldiethylene triaminepentaacetic acid (MX-DTPA) is an exemplary chelating agent for conjugation of radionucleotide to the antibody. See, e.g., WO94/11026, which is herein incorporated by reference in its entirety.

The antibodies disclosed herein for use as therapeutic agents can also be formulated as immunoliposomes. Liposomes containing an antibody are prepared by methods known in the art, such as described in U.S. Pat. Nos. 4,485,045 and 4,544,545, which are each herein incorporated by reference in its entirety. Liposomes with enhanced circulation time are disclosed in U.S. Pat. No. 5,013,556, which is incorporated herein by reference in its entirety.

Particularly useful liposomes can be generated by reverse-phase evaporation using a lipid composition comprising phosphatidylcholine, cholesterol, and PEG-derivatized phosphatidylethanolamine (PEG-PE). Liposomes are extruded through filters of defined pore size to yield liposomes with the desired diameter. Fab′ fragments of the antibody of the present disclosurecan be conjugated to the liposomes using art known methods.

A therapeutically effective amount of an antibody of the disclosure relates generally to the amount needed to achieve a therapeutic objective, such as a binding interaction between the antibody and its target antigen that, in certain cases, interferes with the functioning of the target, and in other cases, promotes a physiological response. The amount required to be administered will furthermore depend on the binding affinity of the antibody for its specific antigen, and will also depend on the rate at which an administered antibody is depleted from the free volume other subject to which it is administered. Common ranges for therapeutically effective dosing of an antibody or antibody fragment of the disclosure may be, by way of nonlimiting example, from about 0.1 mg/kg body weight to about 500 mg/kg body weight. Common dosing frequencies may range, for example, from twice daily to once a week.

Formulations

The therapeutic and diagnostic agents of the present disclosure including compounds, nucleic acid molecules, polypeptides, and antibodies, and derivatives, fragments, analogs and homologs thereof, can be incorporated into compositions, such as pharmaceutical compositions, optionally comprising the membrane active peptides of the disclosure, suitable for use with the mode of administration.

In some embodiments, a composition comprising the therapeutic and/or diagnostic agent, optionally further comprising the membrane active peptides of the disclosure, is formulated for systemic administration. In other embodiments, a composition comprising the therapeutic and/or diagnostic agent, optionally further comprising the membrane active peptides of the disclosure, is formulated for local administration.

In some embodiments, a composition comprising the therapeutic and/or diagnostic agent, optionally further comprising the membrane active peptides of the disclosure, is formulated for parenteral, intradermal, subcutaneous, intravenous, or oral, administration. More typically, a composition comprising the therapeutic and/or diagnostic agent, optionally further comprising the membrane active peptides of the disclosure, is formulated for systemic intravenous administration. Even more typically, a composition comprising the therapeutic and/or diagnostic agent, optionally further comprising the membrane active peptides of the disclosure, is formulated for intra-arterial administration as described herein.

Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid (EDTA); buffers such as acetates, citrates or phosphates, and agents for the adjustment of tonicity such as sodium chloride or dextrose. The pH can be adjusted with acids or bases, such as hydrochloric acid or sodium hydroxide. The parenteral preparation can be enclosed in ampoules, disposable syringes or multiple dose vials made of glass or plastic.

Compositions, such as pharmaceutical compositions, suitable for intravenous administration (e.g., via a catheter system) include carriers such as physiological saline, bacteriostatic water, Cremophor (BASF, Parsippany, N.J.) or phosphate buffered saline (PBS). In all cases, the composition must be sterile and should be fluid to the extent that easy syringeability exists. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as manitol, sorbitol, sodium chloride in the composition. Prolonged absorption of the injectable compositions can be brought about by including in the composition an agent which delays absorption, for example, aluminum monostearate and gelatin.

Oral compositions generally include an inert diluent or an edible carrier. They can be enclosed in gelatin capsules or compressed into tablets. For the purpose of oral therapeutic administration, the active compound can be incorporated with excipients and used in the form of tablets, troches, or capsules. Oral compositions can also be prepared using a fluid carrier for use as a mouthwash, wherein the compound in the fluid carrier is applied orally and swished and expectorated or swallowed. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

For oral administration, the compositions, such as pharmaceutical compositions, may take the form of, for example, tablets or capsules prepared by conventional means with pharmaceutically acceptable excipients such as binding agents (e.g., pregelatinised maize starch, polyvinylpyrrolidone or hydroxypropyl methylcellulose); fillers (e.g., lactose, microcrystalline cellulose or calcium hydrogen phosphate); lubricants (e.g., magnesium stearate, talc or silica); disintegrants (e.g., potato starch or sodium starch glycolate); or wetting agents (e.g., sodium lauryl sulphate). The tablets may be coated by methods well known in the art. Liquid preparations for oral administration may take the form of, for example, solutions, syrups, or suspensions, or they may be presented as a dry product for constitution with water or other suitable vehicle before use. Such liquid preparations may be prepared by conventional means with pharmaceutically acceptable additives such as suspending agents (e.g., sorbitol syrup, cellulose derivatives or hydrogenated edible fats); emulsifying agents (e.g., lecithin or acacia); non-aqueous vehicles (e.g., almond oil, oily esters, ethyl alcohol or fractionated vegetable oils); and preservatives (e.g., methyl or propyl-p-hydroxybenzoates or sorbic acid). The preparations may also contain buffer salts, flavoring, coloring, and sweetening agents as appropriate.

Preparations for oral administration may be suitably formulated to give controlled release of the active compound. For buccal administration the compositions may take the form of tablets or lozenges formulated in conventional manner. For administration by inhalation, the compounds for use according to the present invention are conveniently delivered in the form of an aerosol spray presentation from pressurized packs or a nebuliser, with the use of a suitable propellant, e.g., dichlorodifluoromethane, trichlorofluoromethane, dichlorotetrafluoroethan-e, carbon dioxide or other suitable gas. In the case of a pressurized aerosol the dosage unit may be determined by providing a valve to deliver a metered amount. Capsules and cartridges of e.g. gelatin for use in an inhaler or insufflator may be formulated containing a powder mix of the compound and a suitable powder base such as lactose or starch. The compounds may be formulated for parenteral administration by injection, e.g., by bolus injection or continuous infusion. Formulations for injection may be presented in unit dosage form, e.g., in ampoules or in multi-dose containers, with an added preservative. The compositions may take such forms as suspensions, solutions, or emulsions in oily or aqueous vehicles, and may contain formulatory agents such as suspending, stabilizing, and/or dispersing agents. Alternatively, the active ingredient may be in powder form for constitution with a suitable vehicle, e.g., sterile pyrogen-free water, before use. The compounds may also be formulated in rectal compositions such as suppositories or retention enemas, e.g., containing conventional suppository bases such as cocoa butter or other glycerides. In addition to the formulations described previously, the compounds may also be formulated as a depot preparation. Such long acting formulations may be administered by implantation (for example subcutaneously or intramuscularly) or by intramuscular injection. Thus, for example, the compounds may be formulated with suitable polymeric or hydrophobic materials (for example as an emulsion in an acceptable oil) or ion exchange resins, or as sparingly soluble derivatives, for example, as a sparingly soluble salt.

For administration by inhalation, the compounds are delivered in the form of an aerosol spray from pressured container or dispenser which contains a suitable propellant, e.g., a gas such as carbon dioxide, or a nebulizer.

In one embodiment, the active compounds are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811.

It is especially advantageous to formulate oral or parenteral compositions in dosage unit form for ease of administration and uniformity of dosage. Dosage unit form as used herein refers to physically discrete units suited as unitary dosages for the subject to be treated; each unit containing a predetermined quantity of active compound calculated to produce the desired therapeutic effect in association with the required pharmaceutical carrier. The specification for the dosage unit forms of the disclosure are dictated by and directly dependent on the unique characteristics of the active compound and the particular therapeutic effect to be achieved, and the limitations inherent in the art of compounding such an active compound for the treatment of individuals.

The nucleic acid molecules of the disclosure can be inserted into vectors and used as gene therapy vectors. Gene therapy vectors can be delivered to a subject by, for example, intravenous injection, local administration (see, e.g., U.S. Pat. No. 5,328,470) or by stereotactic injection (see, e.g., Chen, et al., 1994. Proc. Natl. Acad. Sci. USA 91: 3054-3057). The pharmaceutical preparation of the gene therapy vector can include the gene therapy vector in an acceptable diluent, or can comprise a slow release matrix in which the gene delivery vehicle is imbedded. Alternatively, where the complete gene delivery vector can be produced intact from recombinant cells, e.g., retroviral vectors, the pharmaceutical preparation can include one or more cells that produce the gene delivery system. The pharmaceutical compositions can be included in a container, pack, or dispenser together with instructions for administration.

### Methods

In another aspect, the present disclosure is directed to a method of opening a blood-brain barrier. The method typically comprises administering to the subject a composition or combination comprising at least one membrane-active peptide as described herein.

As used herein, the “brain” or “brain parenchyma” refers to the brain and brain stem tissues and any anatomic feature therein, and can include any anatomical region of the brain, such as the cerebrum (composed of the cortex and the corpus callosum), the diencephalon (composed of the thalamus, pineal body, and the hypothalamus), the brain stem (composed of the midbrain, pons, medulla oblongata), and the cerebellum. The brain or brain parenchyma can also include any functional region of the brain, including the frontal lobe, temporal lobe, central sulcus, parietal lobe, and occipital lobe, as well as deep structures of the limbic system, including the limbic lobe, corpus callosum, mammillary body, olfactory bulb, septal nuclei, amygdala, hippocampus, cingulate gyrus, fornix, and thalamus. The term “brain parenchyma” particularly refers to the functional portion of the brain, as compared to features that are merely structural.

As used herein, the “blood-brain barrier” or “BBB” refers to a highly selective permeability barrier that separates the circulating blood from the brain extracellular fluid in the central nervous system (CNS). The barrier is mainly composed of endothelial cells in the brain capillaries and overlying astrocytic foot processes. The endothelial cells are adjoined continuously by tight junctions composed of transmembrane proteins, such as occludin, claudins and junctional adhesion molecules, which limit paracellular diffusion. This system allows the passage of some molecules by passive diffusion, e.g., oxygen, carbon dioxide and hormones. Cells of the barrier also actively transport metabolic products, such as glucose, across the barrier using specific transport proteins.

As used herein, a “compromised blood-brain barrier,” refers to a blood-brain barrier which has been at least partially disrupted. The term particularly refers to a location where the tight junctions between the capillary endothelial cells of the blood-brain barrier have been compromised such that e.g., therapeutic and/or diagnostic agents may pass or diffuse into the brain parenchyma through the compromised tight junctions.

Without being limited by theory, compositions or combinations comprising the membrane-active peptides of the present disclosure are believed to disrupt the cell-cell junctions between the microvascular endothelial cells enabling transient entry into the brain. More particularly, the compositions or combinations comprising the membrane-active peptides of the instant disclosure may induce strain on surrounding cells, which disrupt the cell-cell junctions of the blood-brain barrier resulting in focal leaks.

Typically, the compositions comprising the membrane-active peptides of the present disclosure reversibly disrupt the cell-cell junctions between the microvascular endothelial cells, such that the focal leaks are only transient. In some embodiments, the blood-brain barrier is opened, e.g. is permeable to therapeutic and/or diagnostic agents for less than 6 days, such as 24 hours or less, such as 90 minutes or less, such as 30 minutes or less, such as five minutes or less.

In some embodiments, a composition or combination comprising at least one membrane-active peptide as described herein is administered to a subject by intra-arterial infusion. In some embodiments, intra-arterial infusion is performed using an infusion device, such as a catheter, such as an intra-arterial catheter, in combination with injection of a contrast agent and magnetic resonance imaging (MRI) to visualize the parenchyma territory that is perfused with the at least one membrane-active peptide. This visualized parenchyma territory is expected to correspond to the blood-brain barrier opening as described, for example, in U.S. Patent Application No. 2017/0079581, which is herein incorporated by reference in its entirety.

As used herein, “MRI contrast agents” are a group of contrast media used to improve the visibility of internal body structures in magnetic resonance imaging (MRI). “Magnetic resonance imaging” or “MRI” as used herein refers to a form of medical imaging that measures the response of the atomic nuclei of body tissues to high-frequency radio waves when placed in a strong magnetic field, and that produces images of the brain and/or internal organs. The most commonly used compounds for contrast enhancement are gadolinium-based. MRI contrast agents alter the relaxation times of atoms within body tissues where they are present after e.g., intra-arterial administration. In MRI scanners, sections of the body are exposed to a very strong magnetic field, then a radiofrequency pulse is applied causing some atoms (including those in contrast agents) to spin and then relax after the pulse stops. This relaxation emits energy which is detected by the scanner and is mathematically converted into an image. The MRI image can be weighted in different ways giving a higher or lower signal.

Typical contrast agents for use in the methods, compositions and combinations of the present disclosure include iron oxide nanoparticle formulation, gadolinium and/or Feraheme or a combination thereof, gadoterate, gadodiamide, gadobenate, gadopentetate, gadoteridol, gadoversetamide, gadoxetate, gadobutrol, gadoterate, gadodiamide, gadobenate, gadopentetate, gadoteridol, gadofosveset, gadoversetamide, gadoxetate and gadobutrol.

In some embodiments, MRI is used with a contrast agent during intra-arterial infusion to adjust the rate of the intra-arterial infusion and/or the position of a tip of an infusion device, e.g. the position of a catheter tip, such as an intra-arterial catheter tip, to precisely determine the area of a blood-brain barrier opening as described, for example, in U.S. Patent Application No. 2017/0079581, which is herein incorporated by reference in its entirety.

For example, as taught in 2017/0079581, the perfusion territory, which typically corresponds to the region of the blood-brain barrier opening, may be affected by even small changes in the tip position of an infusion device, such as a catheter, such as an intra-arterial catheter, even within the same vascular territory. Further, faster rates of infusion are generally associated with a larger perfusion territory. Thus, in some embodiments, the locality of an infusion device-driven, e.g., catheter-driven, parenchymal flow may be precisely modulated by varying the infusion rate and the position of a tip, e.g. a catheter tip, using non-invasive MRI in real time to accurately define the brain area in which the blood-brain barrier opens.

Typically, titration of the injection rate is performed on a subject-by-subject basis. Without being limited by theory, variability in blood-brain barrier opening is understood to stem from the highly integrated and collateralized cerebral circulation from the four major cerebral arteries and microregulation dynamically routing cerebral blood flow. This phenomenon has been reported to be a major driver of variability in clinical outcomes with intra-arterial drug delivery into the brain. See, for example, D. S. Liebeskind, Collateral circulation. Stroke 34, 2279-2284 (2003), which teaches that variability in opening territory is likely due to differences in blood supply and collateral flow.

Exemplary rates of infusion can include any suitable infusion rate, such as, 0.01 milliliters (mL)/second (sec). The infusion rate can also include any range from about 0.001 mL/sec, to about 0.005 mL/sec, to about 0.01 mL/sec, to about 0.015 mL/sec, to about 0.02 mL/sec, to about 0.025 mL/sec, to about 0.03 mL/sec, to about 0.035 mL/sec, to about 0.04 mL/sec, to about 0.045 mL/sec, to about 0.05 mL/sec, to about 0.06 mL/sec, to about 0.07 mL/sec, to about 0.08 mL/sec, to about 0.09 mL/sec, to about 0.10 mL/sec, to about 0.20 mL/sec, to about 0.30 mL/sec, to about 0.40 mL/sec, to about 0.50 mL/sec, to about 0.60 mL/sec, to about 0.70 mL/sec, to about 0.80 mL/sec, to about 0.90 mL/sec, or more.

In addition, the length of time of infusion may be adjusted such that the degree of perfusion of the brain parenchyma is optimized, and in turn, the degree of opening of the blood-brain barrier. For example, perfusion may continuously or discontinuously operate for about 0.1 sec, about 0.2 sec, about 0.3 sec, about 0.4 sec, about 0.5 sec, about 0.6 sec, about 0.7 sec, about 0.8 sec, about 0.9 sec, about 1.0 sec, about 1-1.5 sec, to about 1.25-1.75 sec, to about 1.5-2.0 sec, to about 1.75-3.0 sec, to about 2.0-10.0 sec, to about 5.0-30.0 sec, to about 10.0-50.0 sec, to about 20.0-60.0 sec, to about 1-2 min, to about 2-5 min to about 5-10 min, to about 9-25 min, to about 24-50 min, to about 49-150 min, to up to several hours or more.

In still other embodiments, the placement of the tip of e.g. a catheter, such as an intra-arterial catheter, is positioned in an artery, such as a carotid artery, such as an internal carotid artery, such as a cranial artery (e.g. in the Basilar artery) and may be adjusted and/or moved within the artery during MRI visualization to optimize the perfusion into the brain parenchyma, and thus, in turn, optimize the opening of the blood-brain barrier.

In another aspect, the present disclosure is directed to a method of delivering a therapeutic and/or a diagnostic agent to a central nervous system (CNS) of a subject in need thereof comprising: administering to the subject a composition or combination comprising the at least membrane-active peptide and a therapeutic or diagnostic agent as herein described. In some embodiments, the at least one membrane-active peptide and the diagnostic and/or therapeutic agent is in the same composition. In other embodiments, the at least one membrane-active peptide and the diagnostic and/or therapeutic agent are in separate compositions or formulations. It is to be understood that in the methods described herein, the individual components of a co-administration, or combination can be administered by any suitable means, contemporaneously, simultaneously, sequentially, separately or in a single composition.

In some embodiments, a combination comprising at least one membrane-active peptide of the disclosure and a therapeutic and/or diagnostic agent is administered by the same or by a different route of administration. Examples of routes of administration suitable for the at least one membrane active peptide of the disclosure and/or the diagnostic and/or therapeutic agent include parenteral, e.g., intravenous, intradermal, subcutaneous, oral (e.g., inhalation), subdermal, intraperitoneal and intraarterial infusion via e.g., a catheter as described herein. Typically, the combination comprising at least one membrane-active peptide of the disclosure and the therapeutic and/or diagnostic agent are administered intra-arterially as also herein described.

More typically, the present method includes administering a composition comprising at least one membrane-active peptide to an isolated region of the brain via a catheter, thereby opening a region of the blood-brain barrier; and administering a contrast agent as described herein to said isolated region via the catheter; and locating the regional opening in the blood-brain barrier, wherein said locating comprises magnetic resonance imaging as herein described; and then administering another composition comprising a therapeutically effective amount of a therapeutic agent or administering a diagnostic agent to the located regional opening in the blood-brain barrier.

For example, in some embodiments after determining that the blood-brain barrier or a select portion thereof is opened e.g., after visualization of the parenchyma territory that is perfused with a composition comprising the at least one membrane-active peptide using contrast agents in combination with MRI as herein described, a therapeutic and/or or diagnostic agent as also herein described may be administered by intra-arterial infusion, e.g., through the same or a separate catheter, at the site or proximal to the site of the blood-brain barrier opening in a therapeutically effective amount.

As used herein, “a therapeutically effective amount” refers to the amount of a compound that, when administered to a patient for treating a disease is sufficient to effect such treatment, e.g., the amount of such a substance that produces some desired local or systemic effect at a reasonable benefit/risk ratio applicable to any treatment, e.g., is sufficient to ameliorate at least one sign or symptom of a disease, e.g., to prevent progression of the disease or condition, e.g., prevent tumor growth, decrease tumor size, induce tumor cell apoptosis, reduce tumor angiogenesis, prevent metastasis. When administered for preventing a disease, the amount is sufficient to avoid or delay onset of the disease. The “therapeutically effective amount” will vary depending on the compound, its therapeutic index, solubility, the disease and its severity and the age, weight, etc., of the patient to be treated, and the like. Administration of a therapeutically effective amount of a compound may require the administration of more than one dose of the compound.

Diseases and Disorders

In some embodiments, the subject being administered the composition or combination comprising the membrane-active peptide and a therapeutic and/or diagnostic agent is being treated for a disease or disorder. The term “treating” a disease or disorder refers to reversing, alleviating or inhibiting the process of one or more symptoms of such disorder or condition.

As used herein, a “subject” refers to either a human or non-human animal. By “subject” is meant any animal, such as a mammal, including horses, dogs, cats, pigs, goats, rabbits, hamsters, monkeys, guinea pigs, rats, mice, lizards, snakes, sheep, cattle, fish, and birds. Typically, the subject is a human subject.

In some embodiments, the subject is suffering from a disease or disorder. Diseases can include neurological disorders, which can be categorized according to the primary location affected, the primary type of dysfunction involved, or the primary type of cause. The broadest division is between central nervous system (CNS) disorders and peripheral nervous system (PNS) disorders. The Merck Manual lists brain, spinal cord and nerve disorders in the following overlapping categories, all of which are contemplated by the disclosure: brain damage according to cerebral lobe, i.e., frontal lobe damage, parietal lobe damage, temporal lobe damage, and occipital lobe damage; brain dysfunction according to type: aphasia (language), dysarthria (speech), apraxia (patterns or sequences of movements), agnosia (identifying things/people), and amnesia (memory); spinal cord disorders; peripheral neuropathy & other peripheral nervous system disorders; cranial nerve disorders such as trigeminal neuralgia; autonomic nervous system disorders, such as dysautonomia and multiple system atrophy; seizure disorders, such as epilepsy; movement disorders of the central & peripheral nervous system, such as Parkinson's disease, essential tremor, amyotrophic lateral sclerosis (ALS), Tourette's Syndrome, multiple sclerosis & various types of peripheral neuropathy; sleep disorders, such as narcolepsy; migraines and other types of headache, such as cluster headache and tension headache; lower back and neck pain; central Neuropathy (see neuropathic pain); and neuropsychiatric illnesses (diseases and/or disorders with psychiatric features associated with known nervous system injury, underdevelopment, biochemical, anatomical, or electrical malfunction, and/or disease pathology e.g., attention deficit hyperactivity disorder, autism, Tourette's Syndrome & some cases of obsessive compulsive disorder as well as the neurobehavioral associated symptoms of degeneratives of the nervous system such as Parkinson's disease, essential tremor, Huntington's disease, Alzheimer's disease, multiple sclerosis & organic psychosis.)

The present disclosure contemplates treating a proliferative disease in a subject, such as a benign tumor, such as a benign brain tumor, such as cancer, such as brain cancer. In some embodiments, the subject has a brain tumor, such as a benign brain tumor. The following benign brain tumors are contemplated: meningiomas, acoustic neuromas, pituitary tumors, colloid cysts, arachnoid cysts, craniopharyngiomas, choroid plexus papillomas, hemangioblastomas, epidermoid and dermoid tumors.

As used herein a “meningioma” refers to a tumor that develops from the lining of the brain and spinal cord. In some embodiments, the meningiomas are malignant. Symptoms include seizure, headaches and loss of brain function (sensory problems, loss of coordination, etc.).

As used herein “acoustic neuromas” (a.k.a. vestibular schwannomas) refer to tumors arising from a cranial nerve. The tumor is usually benign and slow growing. The most common symptoms are hearing loss, ringing in the ears, vertigo (dizziness), and headaches.

As used herein “pituitary tumors” refer to tumors of the pituitary gland, which produces hormones to regulate the other glands in the body. These tumors may or may not secrete hormones. Often symptoms develop based on the type of hormone secreted. Pituitary tumors represent approximately 10-15% of all brain tumors. They are most common in the third and fourth decade of life, and males and females are equally affected.

As used herein “colloid cysts” refer to benign tumors that only occur in the third ventricle, an area involved with cerebrospinal fluid flow. Tumors in this area can be life threatening by blocking the flow of cerebrospinal fluid, causing a condition called hydrocephalus. Hydrocephalus may cause headaches, nausea, vomiting, and even comas, which can lead to death.

As used herein, “an arachnoid cyst” refers to a sac of cerebrospinal fluid that develops in the brain. Some of these cysts may develop in infancy, but often they are undiagnosed until a head injury occurs. Arachnoid cysts may cause no symptoms for a long time until they are large enough to put pressure on the brain or cause a deformity.

As used herein “craniopharyngiomas” refer to benign tumors located above and behind the pituitary gland. These tumors grow slowly, but can cause vision problems or pituitary dysfunction.

As used herein, “choroid plexus papillomas” refer to benign tumors that occur in the brain's ventricular system from the cells that make spinal fluid.

As used herein, “hemangioblastomas” refer to benign tumors of blood vessels that are often associated with cysts.

As used herein “epidermoid and dermoid tumors” refer to benign tumors containing accumulated left over skin tissue within the head or spinal canal.

In some embodiments, the subject is suffering from a malignant brain tumor including primary malignant brain tumors and metastatic brain tumors. As used herein, “primary malignant brain tumors” refer to those malignant brain tumors arising from glial cells, which are tissues of the brain other than nerve cells or blood vessels. These tumors can grow quickly and be very destructive. Management of these tumors depends primarily on the health of the patient and the location of the tumor. When feasible, treatment typically includes surgical removal followed by radiation and/or chemotherapy.

As used herein, “metastatic brain tumors” refer to those tumors that originate in tissues outside of the brain, followed by metastasis to the brain. Metastatic tumors account for 10-15% of all brain tumors. The most common tumors that spread to the brain are those that originate in the lung, the breast, the kidney, or melanomas (skin cancer).

### EXAMPLES

The examples and other embodiments described herein are exemplary and not intended to be limiting in describing the full scope of compositions and methods of this disclosure. Equivalent changes, modifications and variations of specific embodiments, materials, compositions and methods may be made within the scope of the present disclosure, with substantially similar results.

### Example 1. Materials and Methods

A. Summary of Study Design

The objective of this study was to evaluate membrane-active peptides (also referred to herein as MAPs) for chemically-induced BBBO. Having identified melittin as a leading candidate, the overall strategy was: (1) to determine cell viability to different melittin concentrations and exposure times and use these results to guide identification of dosing for BBBO. Cytotoxicity assays were conducted on induced pluripotent stem cell (iPSC)-derived brain microvascular endothelial cells (n=5) and human cortical neurons (n=3); (2) Assess dynamics of BBBO through measurement of dye leakage and endothelial cell behavior in a tissue-engineered BBB microvessel model. Microvessel experiments were conducted across a range of melittin doses from 3-100 μM·minutes (n=9). (3) To demonstrate pre-clinical efficacy and safety in a mouse model. Time-course magnetic resonance imaging (MRI) and histological assessment of BBBO was conducted at the minimum effective dose.

In vivo experiments were conducted in at least triplicate, and in some cases additional studies were conducted to better understand variability in melittin-induced BBBO (n=3−6). A cohort of n=5 was used to assess long-term safety after the procedure (30 days). Samples sizes were determine based on experience of previous studies. Microvessels and mice were randomly assigned to melittin doses and experimentalists were blinded while analyzing in vitro and in vivo data. Data were excluded if microvessels imaging planes were not in optical focus to adequately conduct analysis or if iPSC differentiations produced endothelial cells with transendothelial electrical resistance below 1500 Ω cm2. Animals were excluded if their death was related to the complications of surgery, including bleeding and hyperanesthesia. Biological replicates are specified in the Brief Description of the Figures.

B. Statistical Analysis

All statistical analysis was performed using Prism ver. 8 (GraphPad). Metrics are presented as means±standard error of the mean (SEM). The principle statistical tests used were a student's unpaired t-test (two-tailed with unequal variance) for comparison of two unpaired groups, a student's paired t-test (two-tailed with unequal variance) for comparison of two paired groups, and an analysis of variance (ANOVA) for comparison of three or more groups. For ANOVA tests, reported p values were multiplicity adjusted using a Tukey test. Linear regression of metrics across experimental parameters was conducted using least squares fitting with no constraints. An F test was used to determine if linear regression produced a statistically significant non-zero slope; p values were not calculatable in the event of perfect fits. An extra sum-of-squares F test was used to compare slopes between linear fits of dose-dependent behaviors. Differences were considered statistically significant for p<0.05, with the following thresholds: *p<0.05, **p<0.01, ***p<0.001.

C. Cell Culture

Induced pluripotent stem cell (iPSC)-derived brain microvascular endothelial cells (dhBMECs) mimic the barrier function of the human BBB. DhBMECs were differentiated as previously reported. The BC1 iPSC line derived from a healthy individual was used for all experiments. After eleven days of differentiation, cells were detached using Accutase (A1110501, Thermo Fisher Scientific), and seeded onto transwells, glass slides, or type I collagen microchannels coated overnight with 50 μg mL⁻¹ human placental collagen IV (Catalog Number (Cat. No.) C5533, Sigma-Aldrich) and 25 μg mL⁻¹ fibronectin from human plasma (Cat. No. F1141, Sigma-Aldrich). Cells have previously been characterized for expression of tight junction proteins, efflux pumps, and nutrient transporters as well as measurements of barrier function including transendothelial electrical resistance (TEER) or solute permeability. Here, cells with TEER of at least 1500 Ω cm2 were used for all experiments. After detachment, cells were cultured in BBB induction medium: human endothelial cell serum-free medium (HESFM) (Cat. No. 11111044, Thermo Fisher Scientific) supplemented with 1% human platelet poor derived serum (Cat. No. P2918, Sigma-Aldrich), 1% penicillin-streptomycin (Cat. No. 15140122, Thermo Fisher Scientific), 2 ng mL⁻¹ bFGF (Cat. No. 233FB025CF, Fisher Scientific), and 10 μM all-trans retinoic acid (Cat. No R2625, Sigma-Aldrich). After 24 hours, cells were further cultured in BBB maintenance medium: HESFM supplemented with 1% human platelet poor derived serum and 1% penicillin-streptomycin. Human cortical neuron HCN-2 cells (CRL-10742) were obtained from ATCC (Manassas, VA, USA). HCN-2 cells were cultured in DMEM (Cat. No. 31600091, Thermo Fisher Scientific), adjusted to contain 1.5 g L⁻¹ sodium bicarbonate, 4.5 g L⁻¹ D-glucose, and supplemented with 10% heat-inactivated FBS (Cat. No. F4135, Sigma-Aldrich), following ATCC recommendations.

D. Peptide Preparation and Exposure

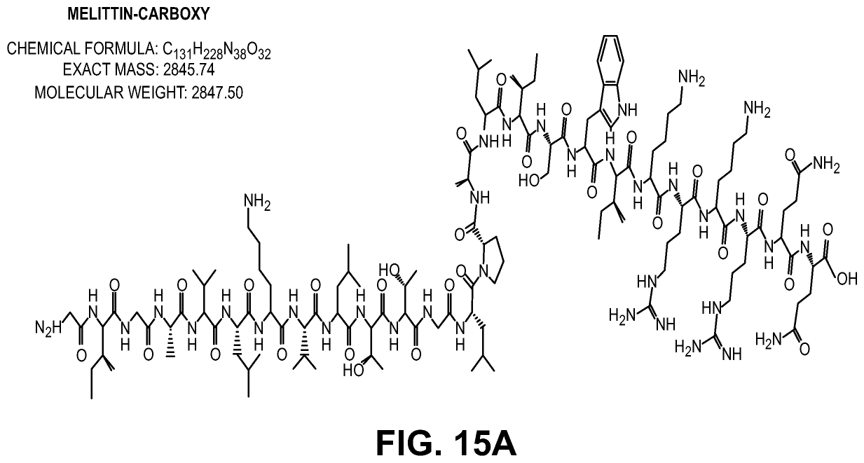

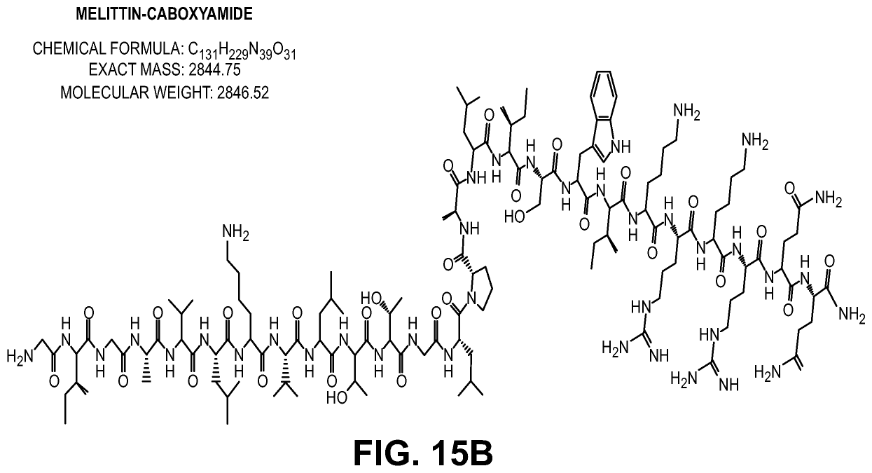

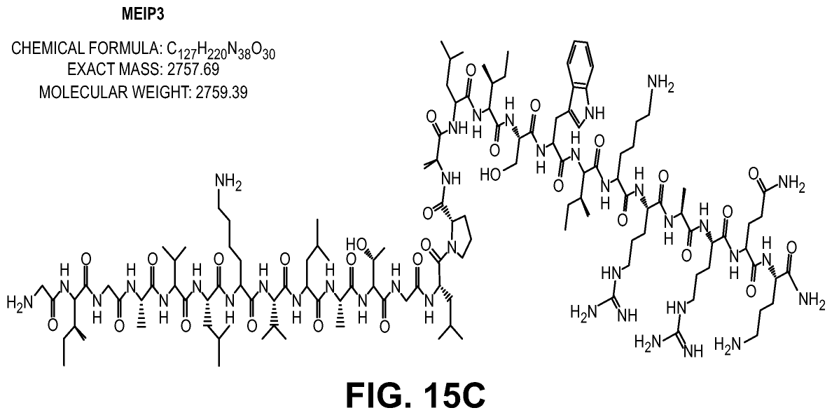

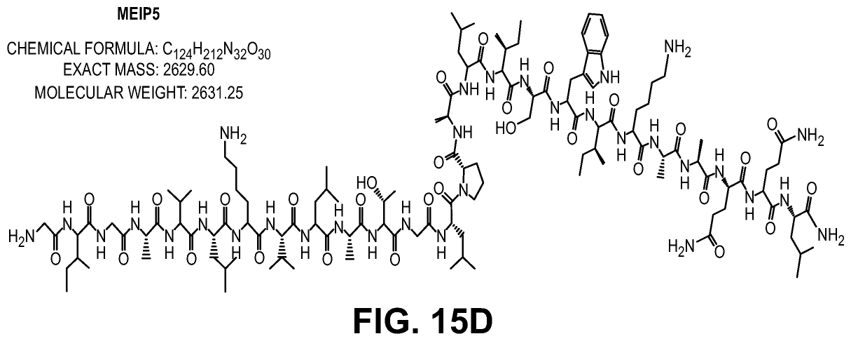

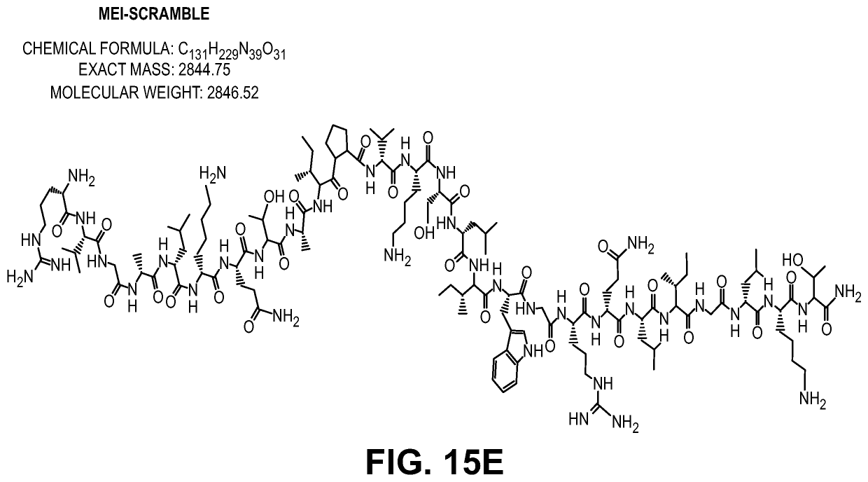

Melittin (melittin-COO—), melittin-CONH₂, scrambled melittin-CONH₂, MelP3, and MelP5 were synthesized at >95% purity by Bio-Synthesis Inc (Lewisville, TX, USA). Peptide sequences and chemical structures are shown in Table 2 and FIGS. 15A-15E. Melittin with a free carboxy C-terminus (melittin-COO—) was used for all experiments unless specified. Peptide stocks were prepared by resuspending lyophilized peptide powders in MilliQ H₂O, following aliquoting and storage at −20° C. Peptide stock aliquots were used for a maximum of three freeze-thaw cycles. Peptide stock solutions were diluted in BBB maintenance medium and prepared fresh prior to experiments.

TABLE 2
Amino acid sequences of selected
melittin variants.

| | | MW |
|---|---|---|
| Peptide | Sequence | (Da) |
| Melittin-COO- | GIGAVLKVLTTGLPALISWIKRKRQQ-OH | 2847.5 |
| Melittin- | GIGAVLKVLTTGLPALISWIKRKRQQ-NH₂ | 2846.5 |
| CONH2^{a} | | |
| MelP3^{a,b} | GIGAVLKVLATGLPALISWIKRARQQ-NH₂ | 2759.4 |
| MelP5^{a,b} | GIGAVLKVLATGLPALISWIKAAQQL-NH₂ | 2631.3 |
| Mel- | RVGALKQTAIPVKSLIWGRQLIGLKT-NH₂ | 2846.5 |
| Scramble^{a,c} | | |

^{a}Variants contain carboxyamide (CONH2) C-terminus.
^{b}Variants discovered in a high-throughput screen for
the ability to have high pore-forming activity in
lipid bilayers.
Amino acids different from wild-type melittin sequence
are bolded.
^{c}Scrambled melittin variant designed to have low
primary and secondary amphipathicity.

E. Transwell Assay

Corning transwell supports (Cat. No. 3470) were coated with 25 μg mL⁻¹ fibronectin and 50 μg mL⁻¹ collagen IV overnight at 37° C. DhBMECs were seeded on transwells in BBB induction medium at 10⁶ cells cm⁻². On day 1 after seeding, the medium was changed to the BBB maintenance medium after one wash with the maintenance medium. On day 2, the TEER for monolayers was measured using EVOM-2 and STX-100 electrode (World Precision Instruments, Sarasota, FL), and monolayers with TEER>1500 Ω cm2 were used for the permeability experiment. TEER of a blank transwell was subtracted from all TEER measurements. The cells were washed with BBB maintenance medium, followed by the addition of 500 kDa dextran-fluorescein (1.2 μM) to the apical side with or without 5 μM melittin. The transwells were incubated while rocking for 90 minutes at 37° C., 5% CO₂. After the permeability experiment, the TEER across monolayers was measured. Dextran fluorescence in the receiving chamber and in the initial apical solution (Cₒ) was measured using the Synergy H4 plate reader (Biotek Instruments Inc., Winooski, VT) and was converted to dextran concentrations using the dextran-fluorescein standard curves. The apparent permeability (Papp) was calculated from Formula 1 below:

$${P}_{app} = \frac{dQ}{dt} \frac{V}{A} \frac{1}{{C}_{o}}$$

where dQ/dt is the increase in dextran concentration in the basolateral chamber, V is the volume of the basolateral chamber, A is the surface area of the transwell support, and Cₒ is the initial dextran concentration in the apical chamber. The permeability experiments were performed with dhBMECs from three independent iPSC differentiations.

F. Immunocytochemistry

Antibodies used in the study are described in Table 3. LabTek 8-chamber slides (Cat. No. 155411, Thermo Fisher Scientific) were coated with 25 μg mL⁻¹ fibronectin and 50 μg mL⁻¹ collagen IV overnight at 37° C. BC1 cell were seeded in BBB induction medium at 5×10⁵ cells cm⁻². On day 1 after seeding, dhBMECs were washed twice with BBB maintenance medium and incubated in the medium overnight. On day 2, the cells were washed twice with BBB maintenance medium and treated with 1.5 μM, 5 μM, and 10 μM of melittin, diluted in the medium, for 2 minutes, 5 minutes, 10 minutes, or 90 minutes. The resulting melittin dosages, calculated as the product of melittin concentration and incubation time, were 3 μM·minutes, 25 μM·minutes, 100 μM minutes, and 450 μM·minutes. A negative control without melittin was included with every experiment. After the melittin incubation, the cells were washed twice with 1×PBS azide and fixed in 3.7% paraformaldehyde solution for 10 minutes or methanol for 15 minutes. The fixed cells were washed with 1×PBS azide and incubated in blocking solution (10% goat serum, 0.3% TritonX-100 in 1×PBS azide) at 4° C. for 1-2 days. The cells were incubated with primary antibodies for 2 hours at room temperature. After the incubation, the cells were washed three times 5-minute each with PBS azide, and incubated with secondary antibodies at 1:200 dilution for one hour at room temperature, followed by three 5-minute washes. Afterwards, the cells were incubated with Fluoromount G, containing DAPI (Cat. No. 00-4959-52, Thermo Fisher Scientific). Epifluorescence images were taken with an inverted microscope Nikon Eclipse TiE using 40×water immersion objective and NIS Advanced Research software (Nikon, Minato, Tokyo, Japan).

TABLE 3
Primary antibodies used in this study.

| Antigen | Description | Vendor | Cat. No. | Dilution |
|---|---|---|---|---|
| Zona occludens-1 | rabbit polyclonal | Life Technologies | 40-2200 | 1:200 |
| Claudin-5 | mouse monoclonal | Life Technologies | 35-2500 | 1:100 |
| Occludin | rabbit polyclonal | Life Technologies | 40-4700 | 1:100 |
| GFAP | rabbit polyclonal | Dako | Z0334 | 1:250 |
| Iba1 | rabbit polyclonal | Wako | 019-19741 | 1:250 |
| Caspase 3 | rabbit polyclonal | Cell Signaling Technology | D175 | 1:400 |
| NeuN | rabbit polyclonal | Cell Signaling Technology | D3S3I | 1:100 |

G. Cytotoxicity Assay

DhBMECs were seeded on polystyrene 96-well plate, coated with 25 μg mL⁻¹ fibronectin and 50 μg mL⁻¹ collagen IV, at 2×10⁵ cells cm⁻² in BBB induction medium. On day 1, the cells were washed twice with BBB maintenance medium with gentle pipetting up-and-down to remove non-adherent cells and incubated in the medium overnight. HCN2 cells were seeded in the tissue culture-treated 96-well plate at 3×10³ cell cm⁻² in HCN2 cell culture medium and cultured for two days at 37° C., 5% CO₂, with the medium change on day 1 after the seeding. The cytotoxicity assay was performed on day 2 after seeding. Briefly, in the cytotoxicity assay, the cells were incubated with two dyes: a membrane-impermeable Ethidium homodimer-1 (EthD1, L3224, Thermo Fisher Scientific) to assess cell viability, and Hoechst 33342 nuclear stain (Cat. No. 62249, Thermo Fisher Scientific) and imaged on an epifluorescence microscope. In more detail, the cells were washed with BBB maintenance medium followed by incubation with 200 μL of 2 μg mL⁻¹ Hoechst 33342 and 10 μM EthD1 solution for 15-30 minutes at 37° C., 5% CO₂. After the incubation, 100 μL of the solutions were replaced with Hoechst 33342 and EthD1 solutions containing melittin using a multichannel pipettor, resulting in final 0.75 μM, 5 μM, and 10 μM melittin concentrations. After the addition of treatment solutions, large 6×6 epifluorescence images were collected every ˜3.5 minutes per condition using a 20×objective and a Nikon Eclipse TiE microscope, equipped with 37° C. chamber, for 60-90 minutes. Out-of-focus portions of images were manually blacked out using ImageJ. Afterwards, the images were analyzed using a custom pipeline in image analysis software CellProfiler 3.1.8 to count Hoechst-positive objects (all cells) and EhtD1-positive objects (dead cells). The fraction of viable cells (F) was calculated using equation 2 below:

$$Fraction viable ( at time i ) = 1 - \frac{{N ( EthD 1 )}_{i} - {N ( EthD 1 )}_{0}}{{N ( Hoechst )}_{max}}$$

where N(EthD1)ᵢ is the number of EthD1-positive objects at time i, N(EthD1)ₒ is the number of EthD1-positive objects at time t=2 minutes (initial time of imaging), N(Hoechst)ₘₐₓ is the maximum number of Hoechst-positive objects counted in an image over the imaging time-course. For dhBMECs, the experiments were repeated a total of five times with two independent iPSC differentiations. In the experiment, all media removal was done by gentle pipetting and not aspiration by vacuum. For HCN2 cells, the experiments were performed a total of three times on day 2 and day 3 after seeding.

H. Three-Dimensional Blood-Brain Barrier Microvessel Fabrication

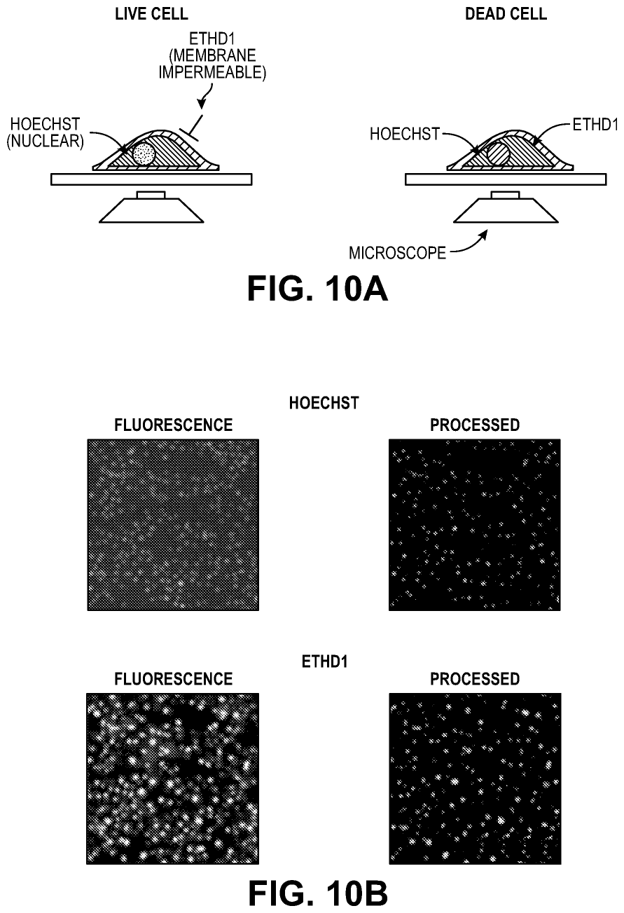

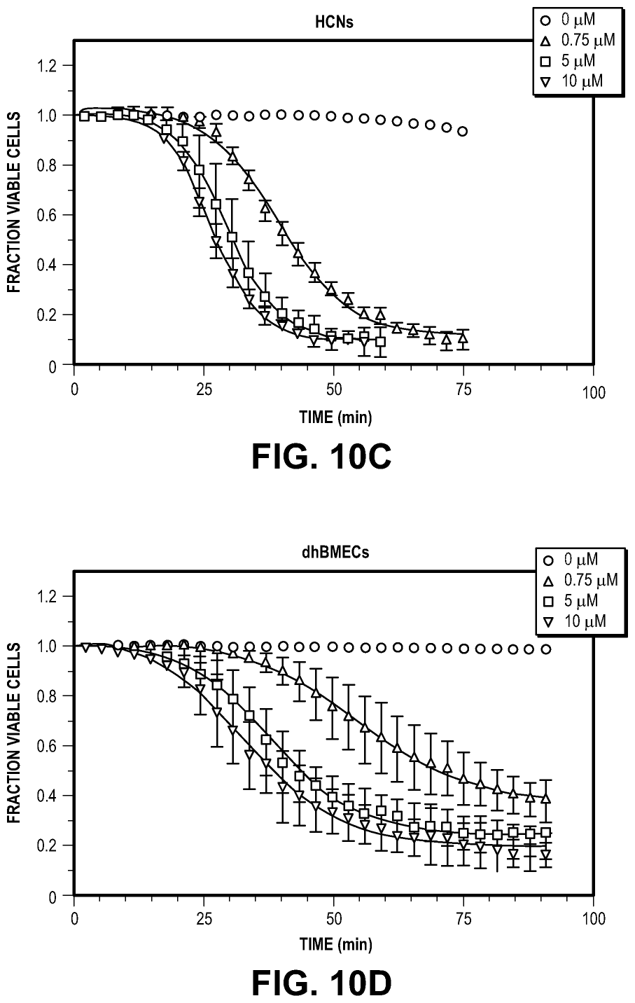

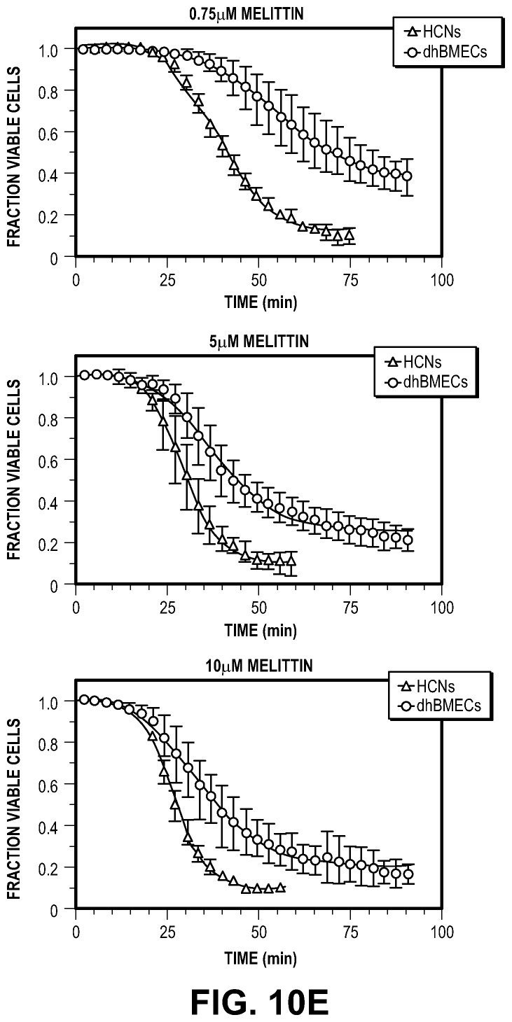

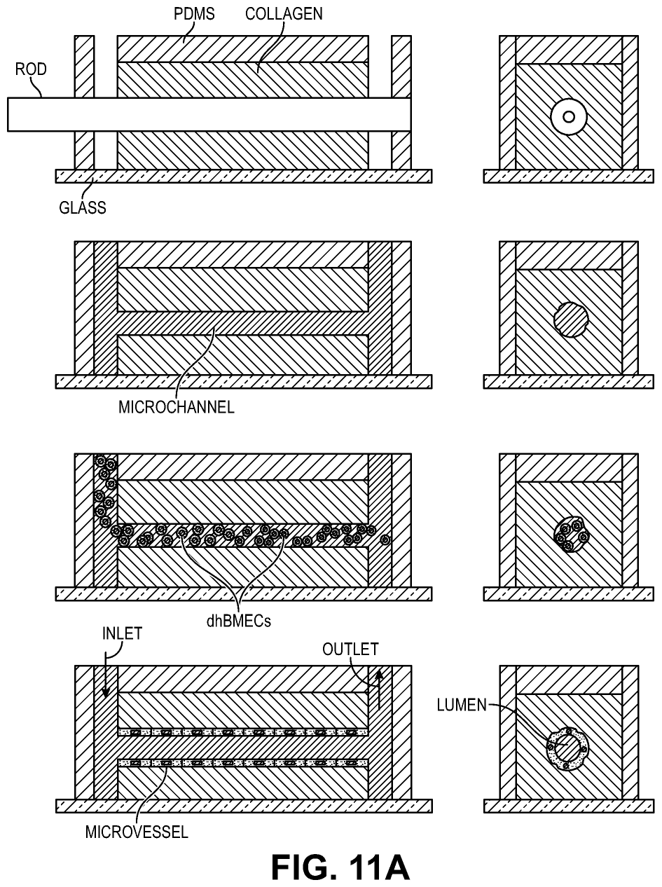

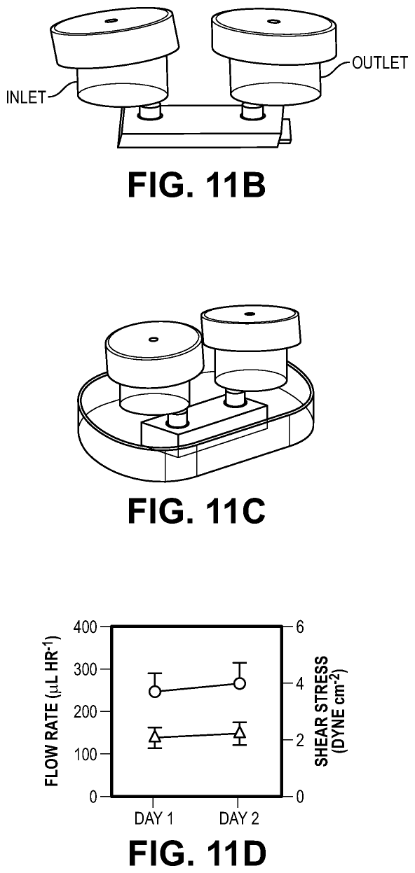

Three-dimensional blood-brain barrier microvessels were fabricated as previously reported (FIGS. 10A and 10B) [Linville, R. M., et al., Biomaterials, 2019. 190-191: p. 24-37] 150 μm diameter and 1 cm long channels were patterned in 7 mg mL⁻¹ type I collagen (Corning) using nitinol wire (Malin Co.) housed within polydimethylsiloxane (PDMS; Dow Corning). Channels were cross-linked by incubation in 20 mM genipin (Wako Biosciences) for 2 hours to increase cell adhesion. 2dhBMECs were suspended in BBB induction medium at a concentration of 5×10⁶ cells mL⁻¹ and seeded into channels. Microvessels were perfused using BBB induction medium for 24 hours and then switched to BBB maintenance medium. During cell seeding and the first day of perfusion, 10 μM ROCK inhibitor Y27632 (ATCC) was supplemented to increase adhesion. An average shear stress of ˜2 dyn cm⁻² was maintained with a reservoir height difference (Δh) of 3 cm, corresponding to a volumetric flow rate of ˜250 μL h⁻¹ (FIG. 11D). This shear stress was estimated from the Poiseuille equation: τ=μQ)/2πd3 where μ is the media dynamic viscosity, Q is the volumetric flow rate, and d is the microvessel diameter.

I. Live-Cell Imaging of Tissue-Engineered Microvessels

Live-cell imaging was conducted to study dynamic changes in in vitro BBB permeability and microvessel structure. Microvessels were imaged using a 10×objective equipped on an inverted microscope (Nikon Eclipse TiE) maintained at 37° C. and 5% CO₂ (FIG. S2C). Epifluorescence illumination was provided by an X-Cite 120LEDBoost (Excelitas Technologies). Phase contrast and fluorescence images were acquired every two minutes with the focal plane set to the microvessel midplane. The entire image collected consists of ten adjacent imaging frames. Microvessels were imaged for 20 minutes before perfusion with fluorescent solutes, and 90 minutes after addition of fluorescent solutes and melittin. A dextran solute panel comprised of 200 μM Cascade Blue-conjugated 3 kDa dextran (Cat. No. D7132. Thermo Fisher Scientific), 16 μM Alexa Fluor647-conjugated 10 kDa dextran (Cat. No. D22914, Thermo Fisher Scientific), and 1.2 μM fluorescein-conjugated 500 kDa dextran (Cat. No. D7136, Thermo Fisher Scientific) was prepared in BBB maintenance medium. Fluorophores were independently excited and the respective emissions were collected using: Nikon UV-2E/C for 3 kDa dextran (50 ms exposure), Chroma 41008 for 10 kDa dextran (20 ms exposure), and Nikon B-2E/C for 500 kDa dextran (50 ms exposure).

J. Analysis of BBB Opening

Image processing was performed using ImageJ (NIH) to quantify BBB opening (FIGS. 11A-11D). First, images were cropped to a standard size of 6 mm×0.6 mm. To determine the spatial heterogeneity of BBB permeability, microvessels were sectioned into 10 regions of interest (ROIs), each a 0.6 mm×0.6 mm square (FIG. 11A). For each ROI, the integrated pixel density was plotted over time (FIG. 11A). The permeability (cm s⁻¹) was calculated using P=(r/2)(1/ΔI)(dI/dt), where r is the vessel radius, ΔI is the increase in fluorescence intensity upon perfusion of the fluorophore, and (dI/dt) is the rate of increase in fluorescence intensity as solute exits into the gel. The average permeability (P_{avg}) was calculated from a linear least squares fit to dI/dt over 90 minutes, while the instantaneous permeability (Pᵢₙₛₜ) was determined from a linear least squares fit to dI/dt in 10-minute increments upon melittin perfusion (FIG. 11A). To visually display permeability dynamics, heatmaps were generated from 90 instantaneous permeability values across the 10 ROIs (x-axis) and nine 10-minute periods (y-axis). To study the dynamics of solute leakage induced by melittin, fluorescence images were converted to binary images using the Auto Threshold function (plugin v1.17; Huang2 method) [Huang, L. K. and M. J. J. Wang, Pattern Recognition, 1995. 28(1): p. 41-51 in ImageJ (FIG. 11B). Under baseline conditions, thresholding results in the lumen designated as white (1) and extracellular matrix (ECM) as black (0). Focal leaks due to melittin exposure appear as white circular plumes into the ECM that emerge (or disappear) over time. From binarized images, individual focal leaks were manually counted over time. Leaks emerging from the sides of the image frame were not counted. To quantify the duration of a BBB opening effect, we identified the time at which the last focal leak appeared and termed the focal leak reversibility time constant (τ_{focal leaks}).

K. Analysis of Cell Behavior

To monitor cell dynamics, cell loss and proliferation events were counted manually. Rates of cell loss (% h⁻¹), division (% h⁻¹), turnover (% h⁻¹), and cell loss at the site of a focal leak (%) were quantified from phase contrast imaging sequences, as reported previously. Briefly, the total number of endothelial cells was counted manually for each microvessel plane, utilizing a region approximately 100 μm wide corresponding to about a fifth of the channel circumference. Cell loss was identified by observing the removal of a cell from the microvessel. Cell loss from the monolayer occurred through two processes: (1) apoptotic loss, in which a cell constricts, releases its contents into the vessel lumen, which is then removed from the monolayer over a period of 20-60 minutes, and (2) non-apoptotic loss, in which a cell swells and disappears from the monolayer over a period of 2-6 minutes. Cell division was identified by observing cell compression, alignment of chromosomes, and the formation of daughter cells. The rate of turnover (% h⁻¹) is the difference between the rate of cell division and the rate of cell loss. A cell loss event was identified to be at the site of a focal leak if it occurred within <50 μm and <4 minutes of the occurrence of a focal leak. For a limited set of experiments, the WTC iPSC line [Kreitzer, F. R., et al., Am J Stem Cells, 2013. 2(2): p. 119-31] with GFP-tagged zona occludens-1 (Allen Cell Institute) was used to facilitate live-cell monitoring of tight junction organization.

L. Confocal Imaging of Tissue-Engineered Microvessels

Confocal z-stacks (0.4 μm in thickness) at 40× magnification were obtained on a swept field confocal microscope system (Prairie Technologies). Illumination was provided by an MLC 400 monolithic laser combiner (Keysight Technologies). Complete reconstruction of the microvessel lumen required approximately four hundred slices. Confocal imaging was conducted before, during, and after exposure to 50 μM·minute melittin treatment.

M. Intra-Arterial Injection of Melittin in Mice

Animal procedures were approved by The Johns Hopkins Animal Care and Use Committee and were performed in accordance with ARRIVE guidelines. The surgical procedures for carotid artery catheterization were performed as described previously [Chu, C., et al., Front Neurol, 2018. 9: p. 921].

Briefly, male C57BL/6J mice (n=43, 6-8 weeks old, 20-25 g, Jackson Laboratory) were anesthetized with 2% isoflurane. The common carotid artery (CCA) bifurcation was exposed and the occipital artery branching off from the external carotid artery (ECA) was coagulated. The ECA and the pterygopalatine artery (PPA) were temporarily ligated with 4-0 silk sutures. A temporary ligature using a 4-0 suture was placed on the carotid bifurcation and the proximal CCA was permanently ligated. A microcatheter (PE-8-100, SAI Infusion Technologies) was flushed with 2% heparin (1,000 U mL⁻¹, heparin sodium, Upjohn), inserted into the CCA via a small arteriotomy, and advanced into the internal carotid artery (ICA).

To explore the optimal concentration of melittin for blood-brain barrier opening (BBBO), 1 μM, 3 μM and 5 μM melittin prepared in saline was infused through the ICA catheter by a syringe pump (11 PLUS, Harvard Apparatus Inc.). For each animal, 150 μL of solution was infused at a rate of 150 μL minute⁻¹. To evaluate BBB status, 0.2 mL of 2% w/v Evans Blue in saline was subsequently administrated through tail vein injection immediately post-melittin infusion and in a separate cohort after 24 hours and 7 days. The brains were harvested immediately after Evans Blue injection, sectioned in brain slicer matrix (Zivic Instruments) to produce 1 mm slabs. After identification of the lowest concentration of melittin that was effective for BBBO, an additional group of animals (n=5) was observed for 30 days to comprehensively assess safety of the procedure. Neurological deficits were assessed by visual inspection to confirm that mice displayed no paralysis, seizure or rotation.

N. MRI and Histological Assessment of BBBO

Animal anesthesia was maintained at 1.5-2% during the MRI scan. Mice were placed on a water-heated animal bed equipped with temperature and respiratory control. Respiration was monitored and maintained at 30-60 minutes⁻¹. All MRI experiments were performed on a Bruker 11.7T MRI scanner. Baseline T2 (TR/TE=2,500/30 ms), susceptibility-weighted imaging (SWI) (TR/TE=378/8 ms), T1 (TR/TE 350/6.7 ms)-weighted, dynamic gradient echo echo-planar imaging (GE-EPI, TR/TE 1250/9.7 ms, field of view (FOV)=14×14 mm, matrix=128×128, acquisition time=60 s and 24 repetitions) and dynamic T1 imaging (TR/TE 2000/3. 5ms, field of view=14×14 mm, acquisition time=8 seconds and 20 repetitions) images of the brain were acquired. The microcatheter was connected to a syringe mounted on an MRI compatible programmable syringe pump (PHD 2000, Harvard Apparatus Inc.). Gadolinium (Gd; Prohance) dissolved in saline at 1:50 was infused intra-arterially at the rate of 150 μL minute⁻¹ under dynamic GE-EPI MRI for visualization of perfusion territory. Then 1, 3 or 5 μm melittin mixed with Gd (50:1) was administered intra-arterially for 1 minute at an injection speed of 150 μL minutes⁻¹. For dynamic monitoring of BBBO, T1 scans were acquired during and after a 1-minute infusion of melittin. For assessment of the BBBO status, T1-weighted image was collected after melittin infusion and repeated at 24 hours and 7 days. To quantify BBB status, T1 intensities in cortex, hippocampus and thalamus/hypothalamus were compared between ipsilateral and contralateral hemispheres. T1 hyperintensity was corrected for possible asymmetry using pre-contrast T1 images and normalized as the ratio of ipsilateral to contralateral intensity. Additionally, the area of BBBO (mm²) was calculated by conversion of T1 images to binary using the Auto Threshold function (plugin v1.17; Maximum Entropy method) [Kapur, J. N., P. K. Sahoo, and A. K. C. Wong, Computer Vision, Graphics, and Image Processing, 1985, 29(3): p. 273-285] in ImageJ. T2-weighted and SWI images were acquired at the same timepoints for detection of brain damage and microhemorrhage. Subsequently, animals were sacrificed for further histological assessment.

To determine the area and region of perfusion territory, we first calculated the contrast enhancement maps at the time where contrast enhancement was at its maximum. As Gd causes negative contrast under T2 imaging, we calculated the contrast enhancement as (SIpre-SIpost)/SIpre, where SI is the signal intensity of an MR image. Histogram was plotted for all pixels in the brain region in a contrast enhancement map and further fitted into two Gaussian distributions, assuming there were two dominant populations for non-contrast enhanced and contrast-enhanced pixels, respectively. The cutoff value to separate the two populations was then determined. The fraction of contrast enhanced areas was calculated by the ratio of the number of pixels, whose values are larger than the cutoff value, to the total number of pixels in the brain. A similar method was used to determine the BBBO territory.

In brief, each post-contrast image was first fitted into two Gaussian distributions, and the cutoff of signal intensity was determined. Data analysis and calculation of area fraction was performed using custom-written MATLAB scripts. 24 hours and 7 days after melittin treatment, animals were trans-cardially perfused with 5% sucrose in deionized water and then 4% paraformaldehyde in phosphate buffered saline. The brains were cryopreserved in 30% sucrose and cryo-sectioned in 30 μm slices. Primary antibodies used in the study are described in Table 2. Goat anti-rabbit Alexa Fluor-488 or Alexa Fluor-594 (1:250, Life Technologies) were used as secondary antibodies for fluorescence assessment using an inverted microscope (Zeiss, Axio Observer Z₁). For demyelination assessment, eriochrome cyanine R staining was performed as described previously [Golubczyk, D., et al., Mol Neurobiol, 2019. 56(8): p. 5740-5748] with assessment using bright-field microscopy. Quantitation of immunohistochemistry (based on relative fluorescence intensity) and MRI analysis (based on intensity) were performed using Image J. For each mouse, three ROIs of the ipsilateral and contralateral BBBO regions were drawn, respectively.

### Example 2. Results

A. Melittin Causes Chemically-Induced BBBO

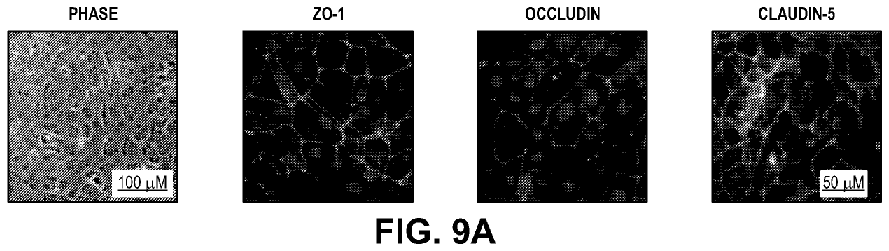

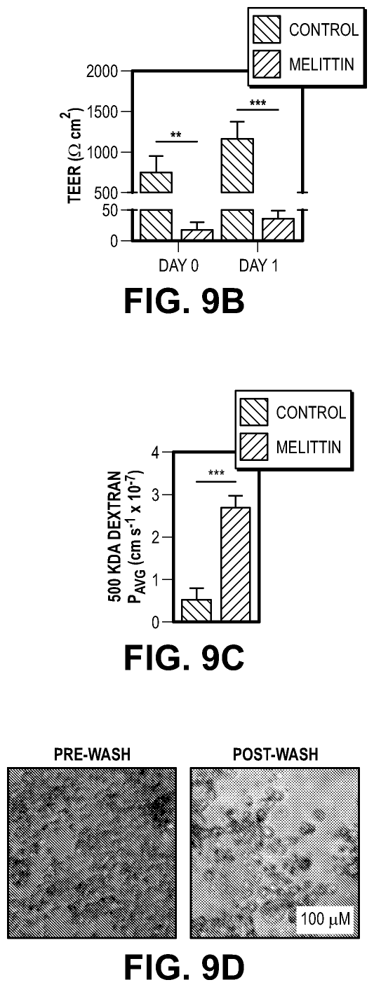

To assess the potential for BBBO, most experiments were performed with melittin with a free carboxy C-terminus (melittin-COO⁻) to, for example, maximize water solubility and hence the accessible concentration range. We subsequently show identical results with natural melittin-CONH₂ as well as two synthetic melittin variants. Melittin induces disruption in confluent monolayers of stem-cell derived brain microvascular endothelial cells (dhBMECs) at concentrations above about 1 μM (FIGS. 9A-9D). In contrast, mannitol induces BBBO above a concentration of about 1.1 M, close to the solubility limit of around 1.4 M. Therefore, even with intra-arterial injection, the concentration window for mannitol-induced BBBO is extremely limited and small decreases in concentration resulting from mixing with the blood due to collateral circulation can affect reproducibility and territories of BBBO. The solubility of melittin is about 350 μM, more than 30-fold larger than the concentrations for membrane activity (1-10 μM), providing a broad window for establishing reversible and non-cytotoxic conditions.

B. Cell Viability in Response to Melittin Dosing

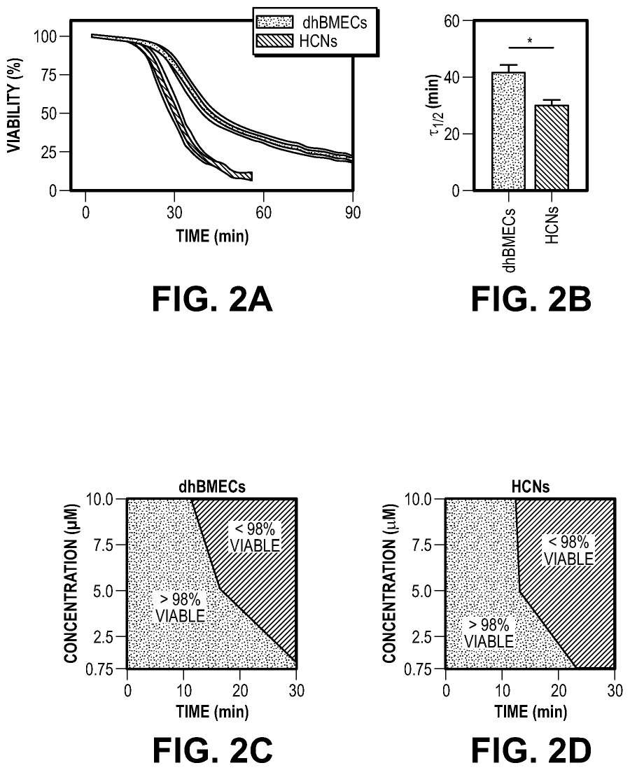

Chemically-induced BBBO relies on exposing brain microvascular endothelial cells (BMECs) to molecules that induce cell stress. Therefore, reversible BBBO is a balance between inducing disruption of cell-cell junctions to enable paracellular transport, while minimizing cell death and maximizing the ability of the BMECs to recover and reform junctions after exposure. We hypothesized that the upper limit of the concentration range for reversible BBBO is dependent, in part, on the limit of viability of BMECs exposed to membrane-active peptides, such as melittin. We first determined the viability of stem-cell derived brain microvascular endothelial cells (dhBMECs) and human cortical neurons (HCNs) exposed to melittin. To accurately assess the limits of viability, we developed a sensitive imaging-based live-cell assay to measure the fraction of ethidium homodimer 1 (EthD1)-positive cells (FIGS. 2A-2D and 10A-10E). The assay consists of time-course imaging of cells in medium containing melittin, nuclear stain (to count all cells), and EthD1 (to count dead cells) and automated image analysis to count the total number of cells and dead cells over time. Upon exposure to 5 μM melittin, the viability of dhBMECs and HCNs is largely unaffected for about 20 minutes, but then decreases sigmoidally (FIG. 2A). This suggests that there is a delay in the cytotoxic or membrane-permeabilizing effect on cells after addition of melittin. The time at which the viability reached 50% (τ_{1/2}) was ˜30 minutes for HCNs compared to ˜40 minutes for dhBMECs (p=0.015) (FIG. 2B). Similarly, we observed shorter τ_{1/2} for HCNs when compared to dhBMECs, across other melittin concentrations (FIGS. 10A-10E). Although cortical neurons appear to be more sensitive to melittin exposure than dhBMECs, in the brain they are shielded from compounds in circulation by the BBB and thus are expected to be exposed to lower melittin concentration than dhBMECs.

To identify the limit of cell viability on exposure to melittin (defined as the highest concentration where the viability is greater than 98%), we constructed phase diagrams (FIGS. 2C, D). The phase diagrams show melittin concentrations and perfusion times, which result in lower or greater than 98% cell viability; n=5 dhBMECs and n=3 for HCNs; statistical significance was calculated by a student's unpaired t-test, * p<0.05; data presented as means±SEM.

High cell viability can be maintained by short exposure (≤10 min) to 10 μM melittin or longer exposure (≥20 min) to 0.75 μM melittin. The relationship between concentration and exposure time that leads to 98% viability is not linear. Decreasing the concentration from 10 μM to 5 μM melittin provides only a slight extension in exposure time for maintaining low cytotoxicity. A short perfusion time is more clinically relevant for intra-arterial injection, and hence we proceeded to investigate the effect of melittin on BBBO for short perfusion times, as they result in low toxicity. Since a dose of 100 μM·minute decreases dhB MEC and HCN viability by <2%, we hypothesized that below this dose, enhancements in permeability could be reversible.

C. BBBO in a Tissue-Engineered Microvessel Model

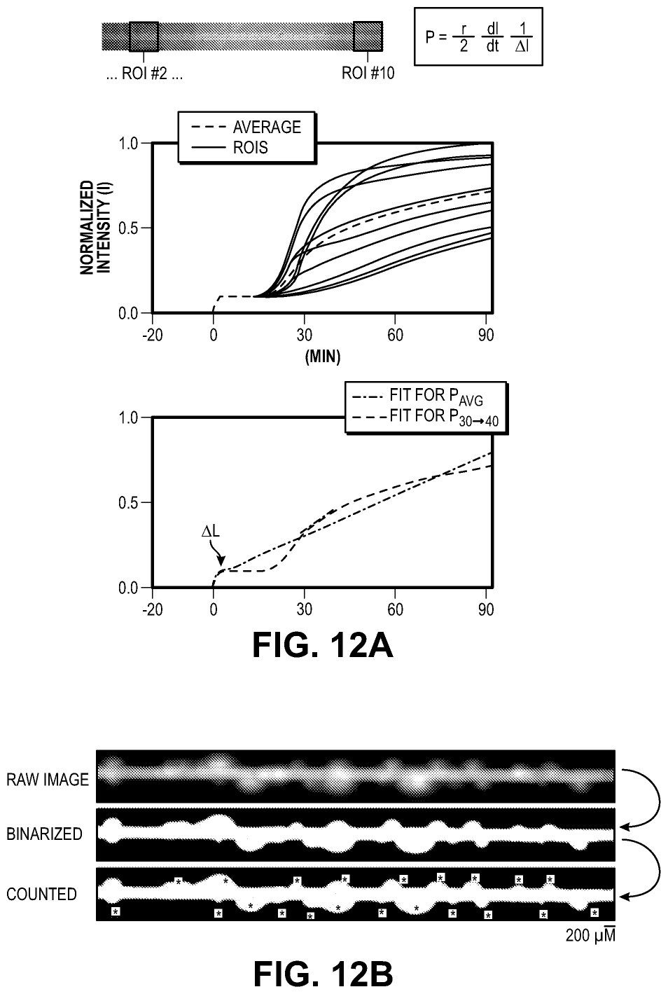

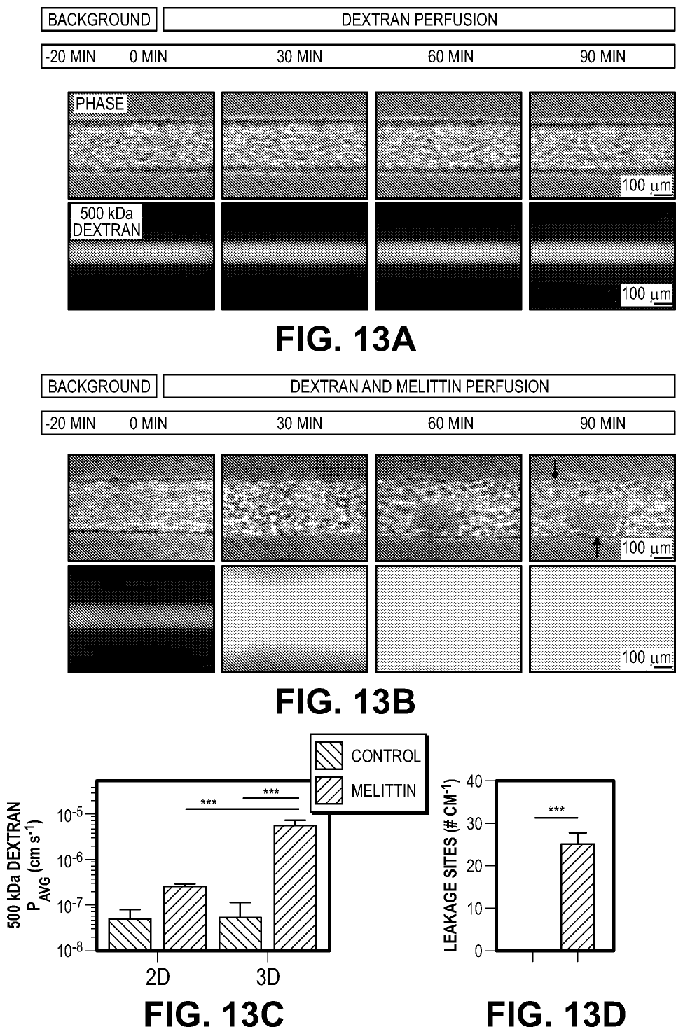

To assess the extent of BBBO and potential for recovery, we used a tissue-engineered BBB model developed in our lab. 150 μm diameter channels patterned in cross-linked collagen I were seeded with dhBMECs and maintained under a shear stress of 2 dyne cm⁻² (FIGS. 11A-11D). Microvessels were perfused with melittin and fluorescently-labeled dextran under live-cell imaging to monitor changes in permeability and cell behavior (FIGS. 12A-12B). This model recapitulates the physiological permeability of the human BBB by restricting paracellular permeability of 500 kDa dextran under baseline conditions. During melittin exposure, barrier function is transiently lost and leakage of 500 kDa dextran is observed (FIGS. 13A-13D).

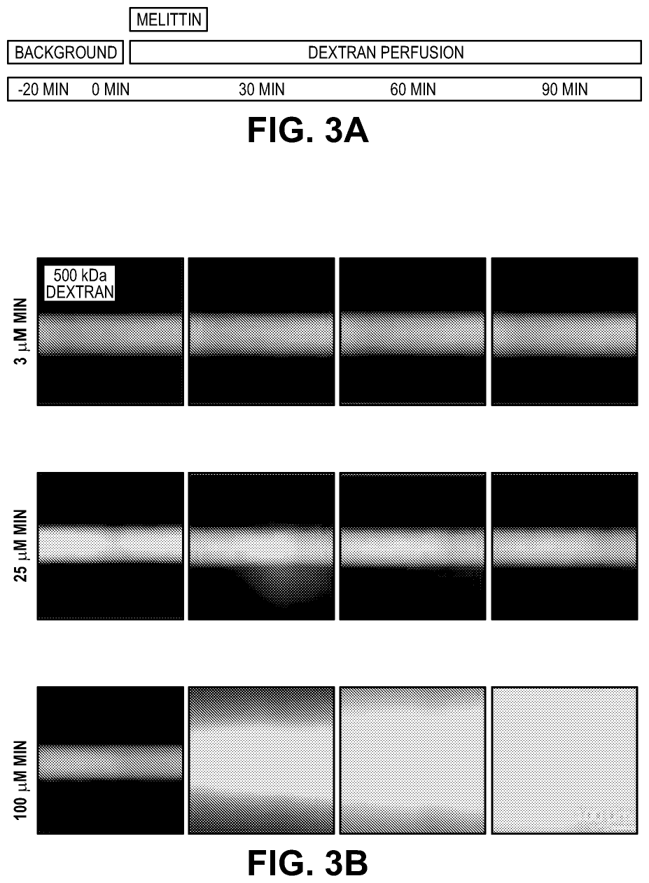

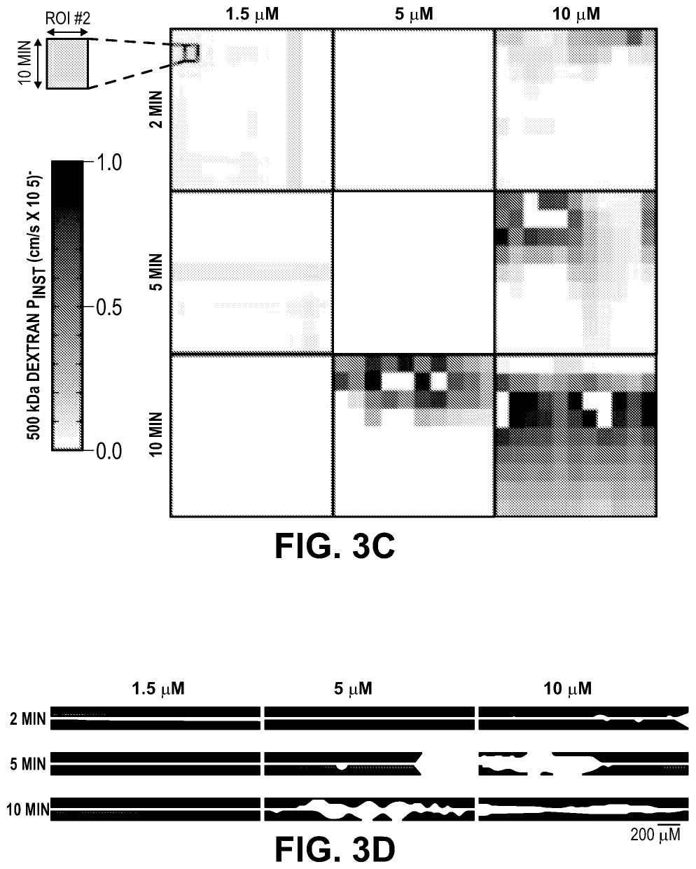

Having identified a melittin dose of 100 μM·minute as an upper limit to maintain neuronal and endothelial viability, we perfused BBB microvessels with various melittin concentrations (1.5, 5 and 10 μM) for different exposure durations (2, 5 and 10 minutes), representing a wide range of doses from 3 to 100 μM·minute (FIGS. 3A-3D). To monitor changes in barrier function, dyes were perfused during and after melittin dosing for a total of 90 minutes (FIG. 3A). Low doses of melittin did not induce BBBO as evident from images recorded during perfusion with 500 kDa dextran, which resembled those of control BBB microvessels (FIGS. 3B and 13A-13D). Intermediate doses of melittin induced BBBO and resulted in recovery during the 90 minute imaging period (FIG. 3B). High doses of melittin induced sustained BBBO, but did not recover during the 90 minutes of imaging (FIG. 3B), although barrier function was regained 24 hours later.

To visualize the spatial and temporal heterogeneity of BBBO, heatmaps were generated for each melittin dose (FIGS. 3C and 12A-12B). The images were split into 10 regions of interest (ROI) along the length of the microvessel. Heatmaps were comprised of 90 instantaneous permeabilities of 500 kDa dextran at different locations along the microvessel length (ROI 1-10, x-axis) and in ten-minute increments (y-axis). These results show that: (1) melittin-induced BBBO is heterogenous in space and time, and (2) the extent and duration of BBBO is dose dependent.

Intermediate and large doses of melittin resulted in the formation of punctate focal leaks, similar to those observed during continuous melittin exposure (FIGS. 3B and 13A-13D). Imaging revealed that the threshold dose for inducing focal leaks was 20 μM·minute (FIG. 3D). At intermediate doses (20-50 μM·minute), focal leaks were transient indicating highly reversible BBBO.

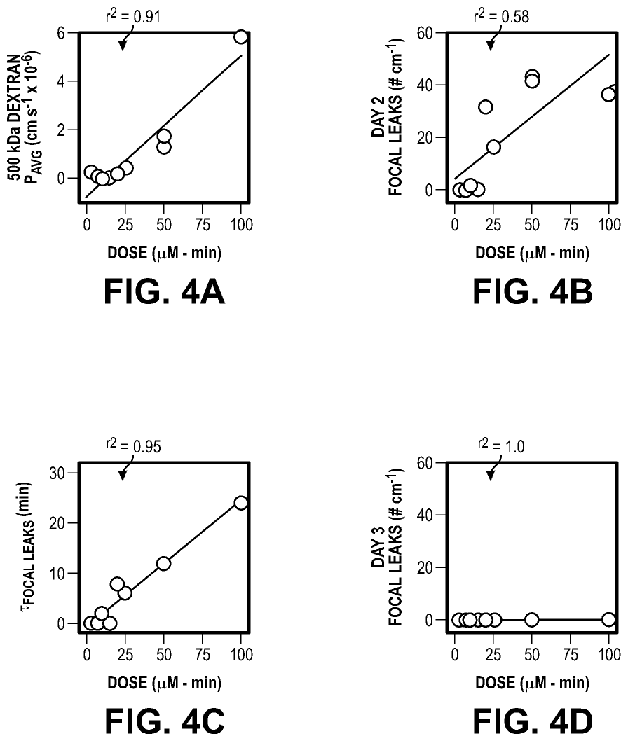

While focal leak formation resulted in heterogeneous spatial and temporal permeability along microvessels, the average permeability of 500 kDa dextran in melittin-exposed microvessels, which is a measure of the extent of opening, was dose dependent (FIG. 4A). The melittin dose (in μM·minute) is defined as the product of melittin concentration and melittin perfusion time. There was a strong positive linear relationship between exposure time and average permeability (r2=0.91, p<0.001 significantly non-zero slope). For example, 50 μM·minute doses increased the average permeability of 500 kDa dextran in a 200 μm segment to approximately 1.5×10−6 cm s-1, ˜25-fold higher than baseline. Additionally, the focal leak density was dose dependent (FIG. 4B) (r2=0.58, p=0.017 significantly non-zero slope). At intermediate melittin doses, there was a linear relationship between the dose and focal leak density, however, at high melittin doses a similar focal leak density was observed. The linear increase in dextran permeability and the plateau in focal leak density suggested that BBBO is not only related to the number of disruption sites but also the size and duration of opening at each site. Focal leaks typically emerged immediately after melittin administration; however, at longer exposure times focal leaks were also observed during dosing.

The duration of BBBO is an important metric to guide drug delivery strategies since it provides a window of opportunity for enhancing drug delivery. As a metric for the duration of opening we identified the time at which the last focal leak appeared, which is defined as the focal leak reversibility time constant (τfocal leaks). There was a strong linear relationship between melittin dose and τfocal leaks (r=0.98, p<0.001 significantly non-zero slope) (FIG. 4C). Additionally, for clinical use, BBBO must be reversible with recovery of normal BBB function after drug delivery. We have previously shown that dhBMEC microvessels display stable permeability over six days with no focal leaks observed (Linville et al., Biomaterials 190-191, 24-37 (2019)).

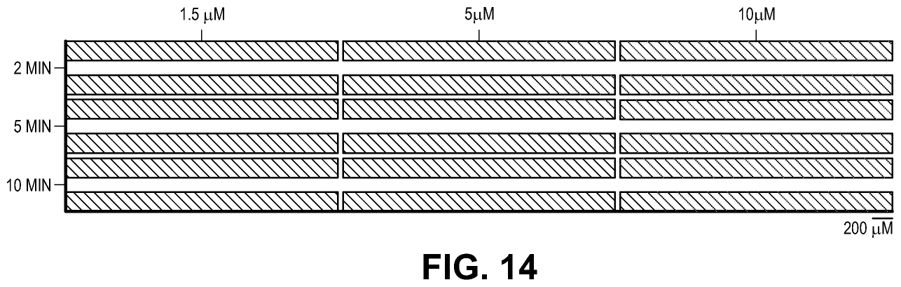

To confirm that melittin-induced BBBO was fully reversible, microvessels were perfused with 500 kDa dextran 24 hours after melittin exposure. For all melittin doses (3-100 μM·minute range), focal leaks were not observed, indicating complete recovery of barrier function (FIG. 4D and FIG. 14).

D. Melittin Variants also Support Reversible BBBO and Highlight Possible Structure Function Relationships Required for Activity

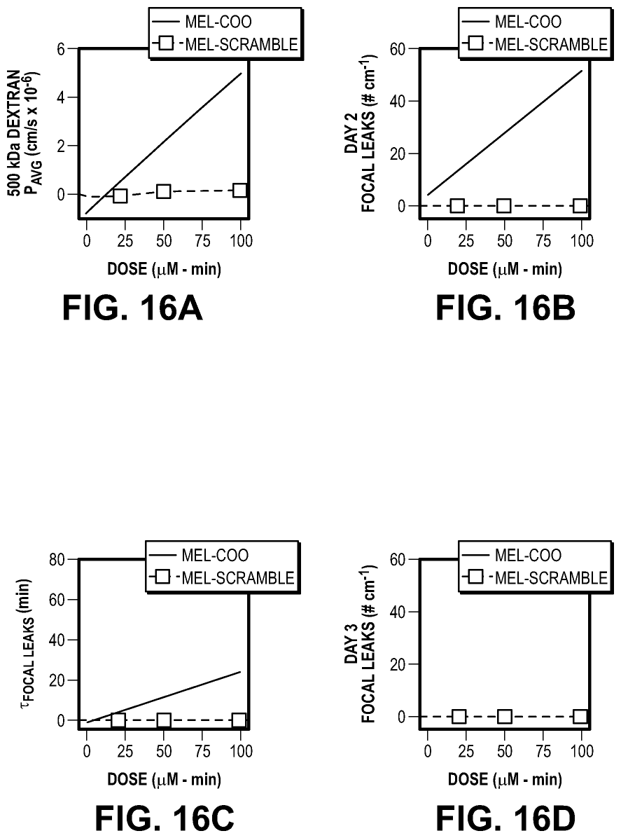

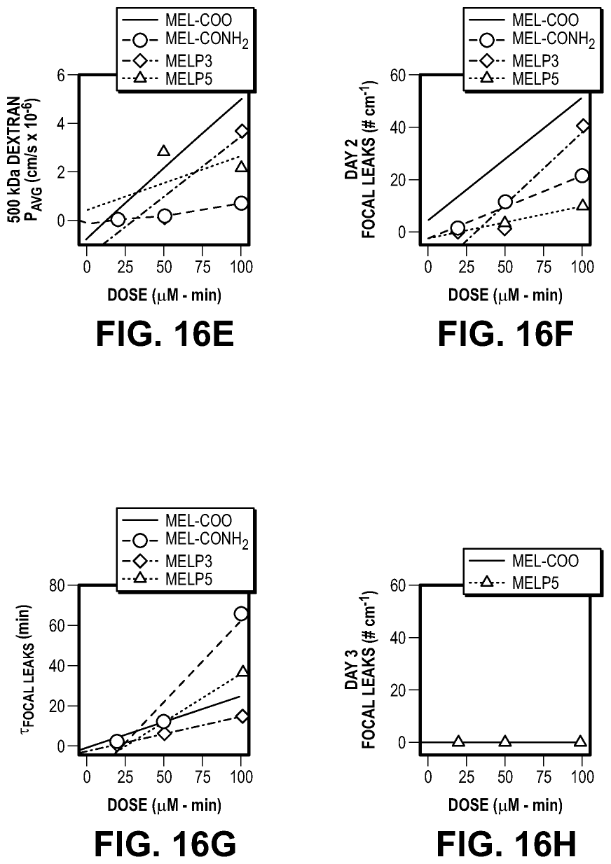

To determine whether the effects of melittin on BBBO are sequence-specific, we characterized the activity of a scrambled version of melittin (Mel-Scramble) (Table 2, below and FIGS. 15A-15E). The melittin structure contains a high number of cationic amino acids (R, K) at the carboxy terminus contributing to its primary amphipathicity. In addition, melittin is largely alpha helical with hydrophobic and charged amino acids separated on different faces, giving it secondary amphipathicity. We redistributed the amino acids in Mel-Scramble to have no primary amphipathicity and minimal secondary amphipathicity. Mel-Scramble did not induce BBBO across 25, 50, and 100 μM·minute doses. There was no change in the permeability of 500 kDa dextran following perfusion with Mel-Scramble and no focal leaks were observed during or 24 hours after perfusion (FIGS. 16A-16H). There was a non-statistically significant relationship between dose and 500 kDa dextran permeability (p=0.335), focal leak density (p=not calculable), and focal leak reversibility (p=not calculable) (FIGS. 16A-16D).

Having established that melittin works in a sequence-specific manner, we assessed the effects of small sequence modifications on BBBO. In particular, we assessed the ability of melittin sequence variants that are known to be membrane active (i.e. Melittin-CONH₂, MelP3, and MelP5) to induce reversible BBBO in microvessels (Table 1 and FIGS. 15A-15E). Thus far, all BBBO experiments were performed with melittin containing a carboxy terminus (COO—). The melittin-CONH₂ variant is a natural form of melittin in bee venom with a carboxyamide (CONH₂) terminus.

At physiological pH, the carboxyamide is neutral and can facilitate insertion into lipid bilayers, contributing to enhanced membrane binding compared to the carboxy terminus variant. MelP3 and MelP5 were originally discovered in a high-throughput in vitro screen for their potent ability to form pores in lipid membranes (Krauson et al., J Am Chem Soc 134,12732-12741 (2012)).

We observed that these three melittin variants at a concentration of 10 μM and at 2-, 5- and 10-minute exposures induced reversible BBBO (FIG. 16E). Across the closely related four variants, there were non-statistically significant differences in the dose-dependence of 500 kDa dextran permeability (p=0.051) and focal leak density (p=0.415) (FIG. 16E, 16F). However, the reversibility of focal leaks was variant-dependent as there were statistically significant differences in the slope of focal leaks versus dose (p<0.001) (FIG. 16G). Specifically, melittin-CONH₂ displayed the highest slope (FIG. 16G), suggesting that disruptions in barrier function may occur over longer time periods than for melittin-COO—, despite less overall disruption (FIG. 16E). We hypothesized that the neutral C-terminus of melittin-CONH₂ had greater binding to dhBMEC membranes, resulting in extended barrier disruption. Across all variants, focal leaks were not observed 24 hours after dosing, suggesting that barrier disruption was reversible (FIG. 16H).

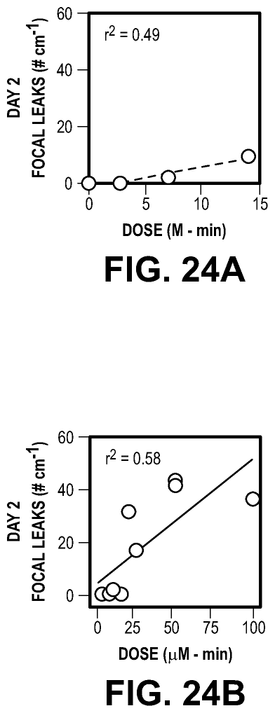

Additionally, we conducted a retrospective analysis to compare mannitol and melittin BBBO within the tissue-engineered BBB microvessels (FIG. 24A and 24B). For both agents a threshold dose must be exceeded to induce opening; for mannitol this threshold was 7 M·minutes, while for melittin it was 20 μM·minutes. Melittin may be able to more potently alter BBB permeability, as above these thresholds melittin resulted in a 3-fold higher focal leak density.

E. Transient Melittin Dosing Decreases Cell Turnover

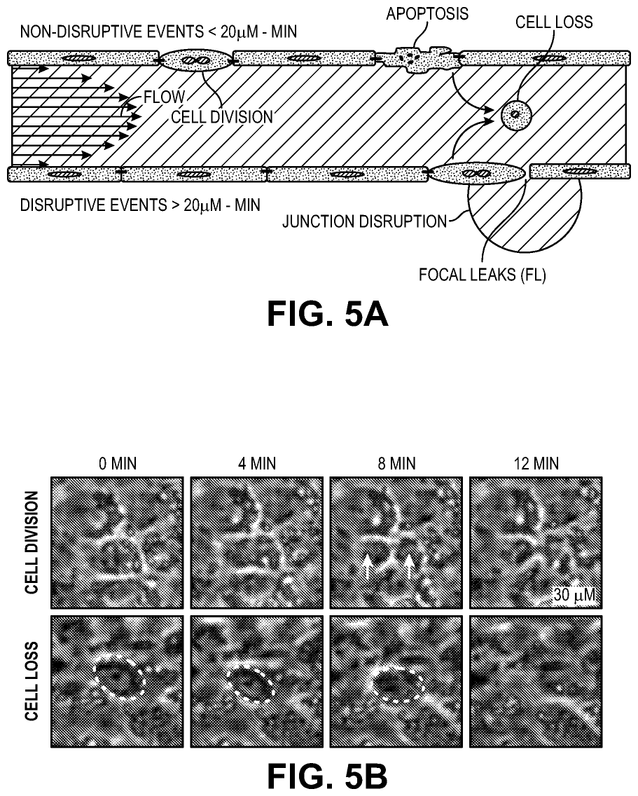

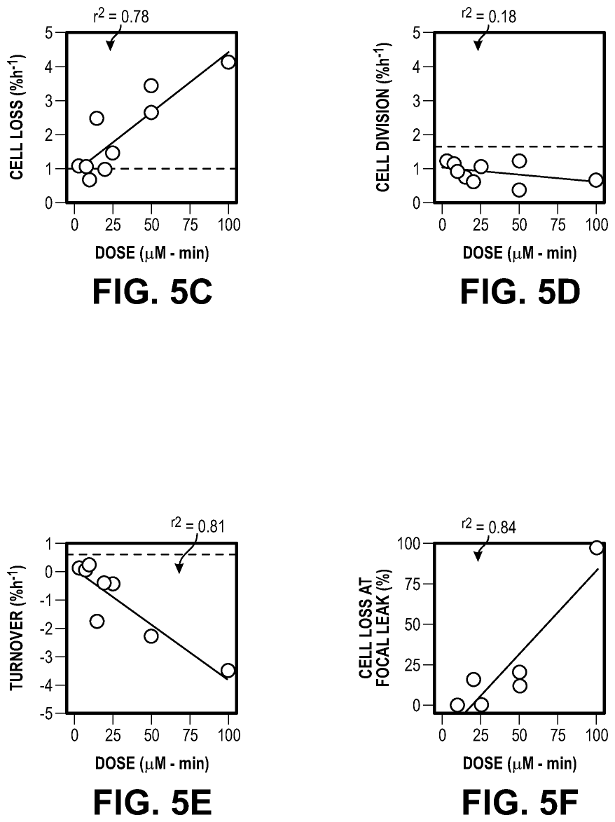

To explore the mechanisms of melittin-induced BBBO, we quantified dhBMEC behavior in microvessels using live-cell imaging (FIG. 5A). At melittin doses below 20 μM·minute, non-disruptive events including cell division and apoptosis were observed. These events were not associated with loss of barrier function or formation of focal leaks under baseline conditions as surrounding endothelial cells are able to dynamically retain cell-cell contacts. However, at melittin doses above 20 μM·minute, junctional disruption was observed resulting in the formation of focal leaks.

To evaluate the effect of melittin dosing on dhBMEC behavior, we manually traced cell loss and cell division events in phase contrast images (FIG. 5B). Under baseline conditions, the loss of cells from dhBMEC microvessels was previously found to be ˜1% h⁻¹. The cell loss rate following melittin exposure was dose-dependent (r²=0.78, p=0.002 significantly non-zero slope) (FIG. 5C). However, we did not detect focal leaks when the cell loss rates were similar to baseline conditions, suggesting that apoptosis can occur without disruptions in barrier function. The division rate was about 1% h⁻¹ but was not melittin dose-dependent (r²=0.18, p=0.260 not significantly non-zero slope) (FIG. 5D), and was lower than under control conditions (˜1.7% h⁻¹), suggesting that melittin inhibits proliferation across tested melittin doses. The turnover of dhBMECs in microvessels, defined as the cell division rate minus the cell loss rate, was negative and displayed a strong dose dependence (r²=0.81, p<0.001 significantly non-zero slope) (FIG. 65E). Under baseline conditions, BBB microvessels displayed a turnover of ˜0.6% h⁻¹, suggesting a net gain of cell density over time. The strong correlation between cell turnover and dose suggested that mechanisms related to cell loss may be responsible for BBBO. Interestingly, the loss of cells from microvessels was spatially correlated with focal leaks (r²=0.84), further suggesting a shared mechanism (FIG. 5F).

F. Transient Melittin Dosing Increases Paracellular Permeability via Disruption of Cell Junctions

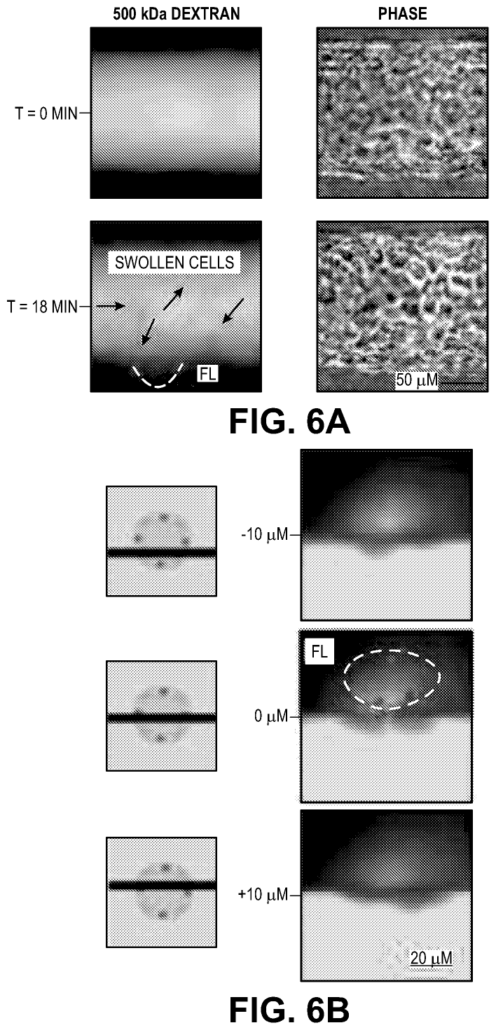

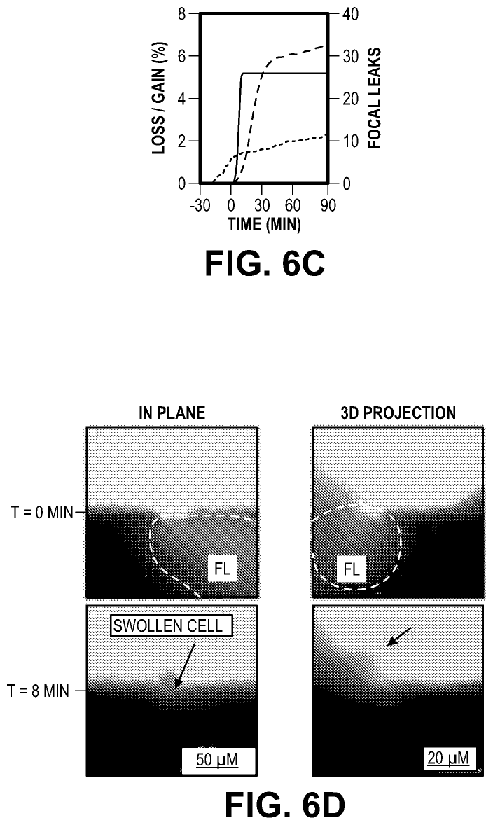

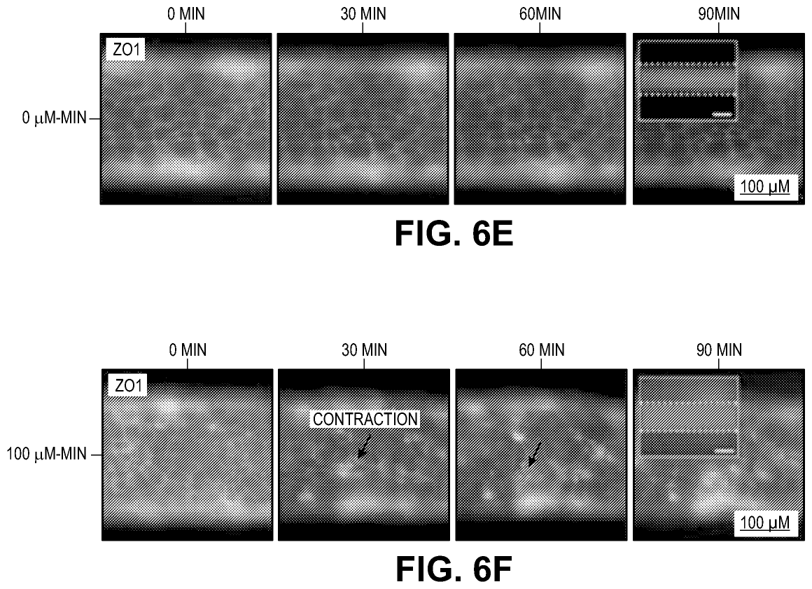

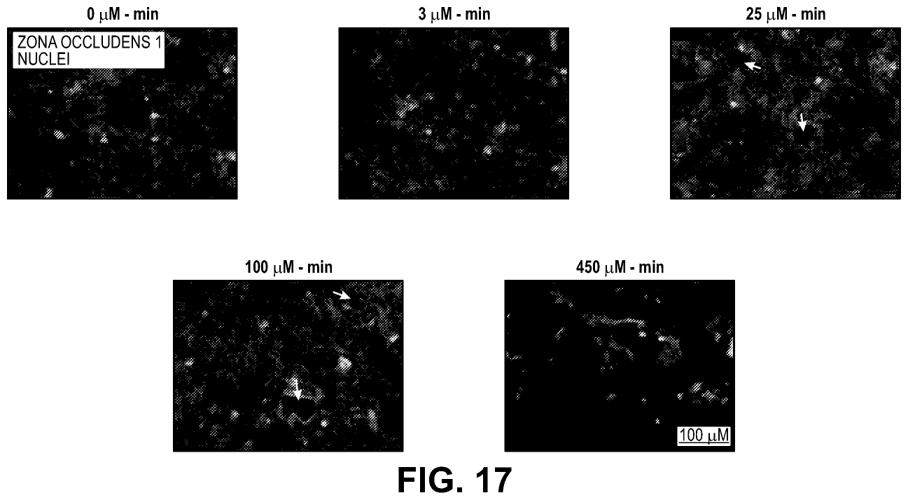

We further investigated the mechanism of melittin-induced BBBO using epifluorescence and confocal microscopy. Epifluorescence images at the microvessel mid-plane showed that melittin exposure resulted in cell swelling, as revealed by the exclusion of 500 kDa dextran (FIG. 6A, (white arrows)). Dextran exclusion from the endothelium suggested that leakage was due to disruption of paracellular barrier function. This was confirmed using confocal microscopy (FIG. 6B). Disruption was spatially localized as the endothelium was intact above and below each site, suggesting that focal leaks originated from discrete disruptions of cell-cell junctions. To further assess this hypothesis, we performed immunohistochemistry for the tight junction (TJ) scaffolding protein zona occludens-1 (ZO1) after melittin treatment of 2D dhBMEC monolayers across doses from 3 μM·minute to 450 μM·minute (matching the doses used in the cell viability assay). Melittin doses of 3-100 μM·minute, within the range of reversible BBBO, did not result in large changes in ZO1 protein expression compared to controls (FIG. 17). However, above the limit of reversibility at a 450 μM·minute dose, significant cell detachment was observed, similar to the results from the viability assay and microvessels at high melittin doses (FIGS. 10A-10E and 13A-13D). Interestingly, cells that bordered the defects in the monolayer frequently showed ZO1 staining (FIG. 17; marked with white arrows). Thus, we hypothesized that BBB disruption occurs by mechanisms related to local disruption of TJs rather than a global decrease in TJ integrity.

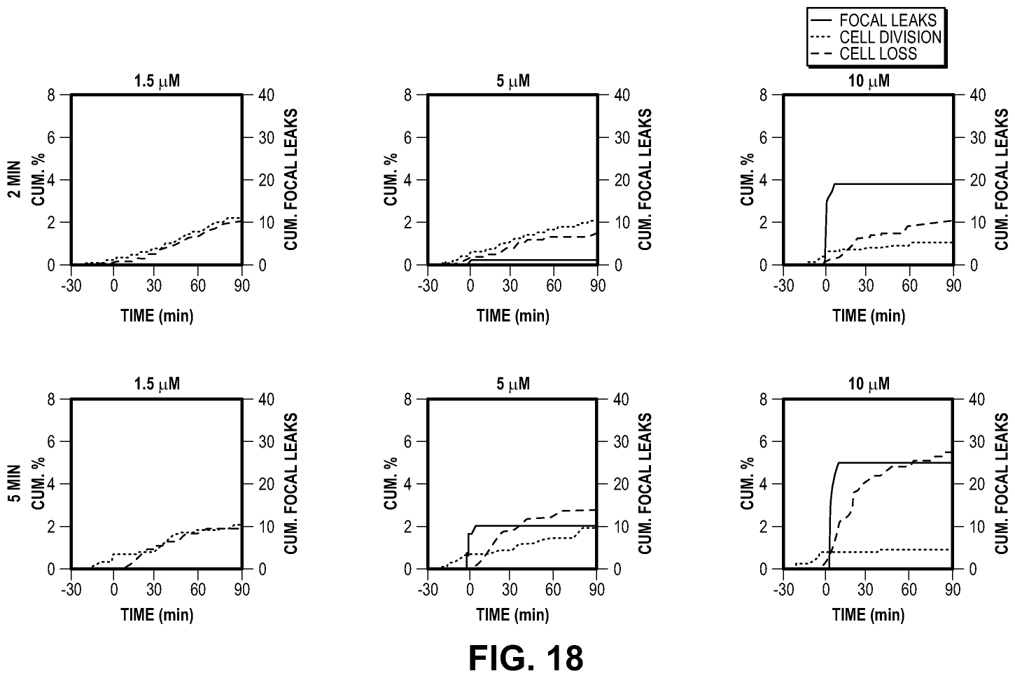

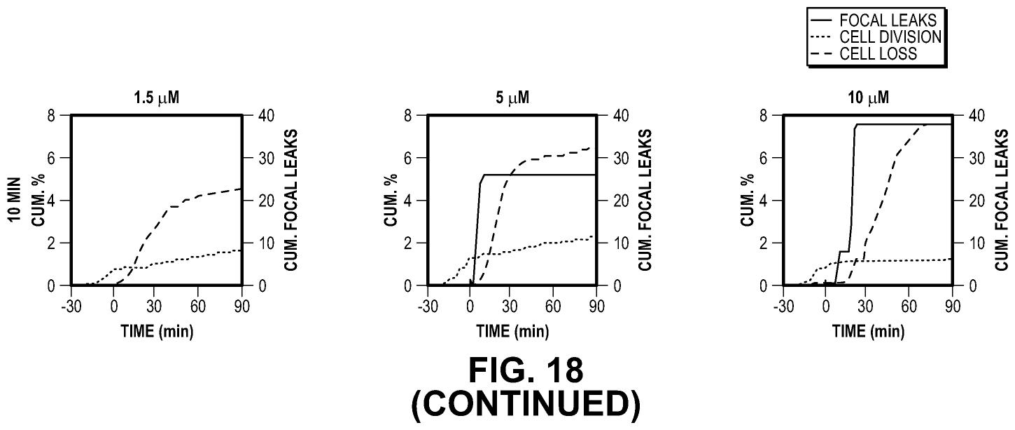

To better understand if focal leak formation during melittin exposure was associated with cell loss, we plotted the cumulative number of cell loss events, cell division events, and focal leaks over two hours following dosing in microvessels (FIG. 18). For a representative 50 μM·minute dose, the number of cell loss events increased over background after a plateau in the number of focal leaks (FIG. 6C). Across other melittin doses, increases in cell loss were consistently observed after focal leak formation (FIG. 18). Thus, the removal of a cell from the endothelium was not responsible for focal leak formation. Instead, time-course confocal imaging showed that focal leaks emerged before cell swelling and with high spatial confinement (FIG. 6D).

To understand the process that leads to the formation of focal leaks, we performed additional experiments on microvessels comprised of dhBMECs with GFP-tagged zona occludens-1 (ZO1). This cell line allowed us to visualize tight junction localization during imaging. Under baseline conditions (no melittin), tight junctions maintained stable localization over 90 minutes with no leakage of 10 kDa dextran (FIG. 6E). However, upon exposure to high melittin doses (100 μM·min), cells contracted, as evident by condensation of ZO1 at discrete locations in the monolayer (FIG. 6F). We speculate that melittin induced cell contraction which transiently disrupted cell-cell junctions and resulted in the formation of focal leaks. We observed that cells that swelled into the microvessel lumen following contraction were eventually removed by the shear flow. These results are consistent with the observed spatial correlation between the loss of cells and focal leaks (r²=0.84) (FIG. 5F).

G. Profile of Melittin-Induced BBBO In Vivo

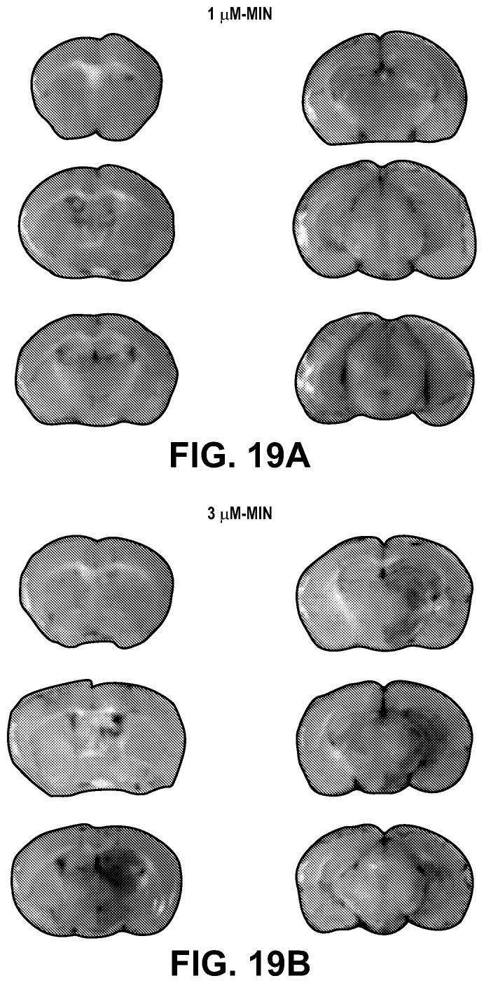

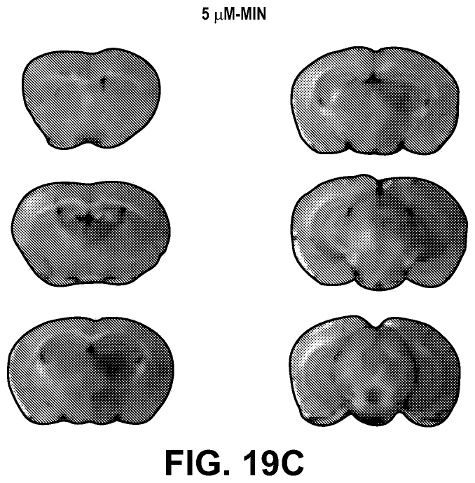

To assess BBBO in vivo, melittin was infused into an internal carotid artery in a mouse model following catheterization. The internal carotid artery predominately supplies the ipsilateral hemisphere of the mouse brain, while other catherization protocols could be used to distribute BBB downstream of any major human artery. In preliminary experiments, we studied three intra-arterial administrations: 1, 3, or 5 μM melittin for 1 minute (1-5 μM·minute). To determine the minimum effective dose, we perfused Evans Blue immediately following the injection to determine BBB status. 3 μM·minute melittin was identified as the minimum effective dose by reproducible leakage of Evans Blue within the downstream ipsilateral hemisphere following injection (FIGS. 19A-19C). No leakage of Evans Blue was observed following 1 μM·minute melittin, indicating that intra-arterial administration itself does not compromise BBB status as previously found. Widespread leakage of Evans Blue was observed following a 5 μM·minute melittin dose, however, mice displayed rotational behavior indicating neurological damage to one hemisphere of the cortex. Thus, 3 μM·minute melittin was further explored for efficacy and safety using MRI and histopathology.

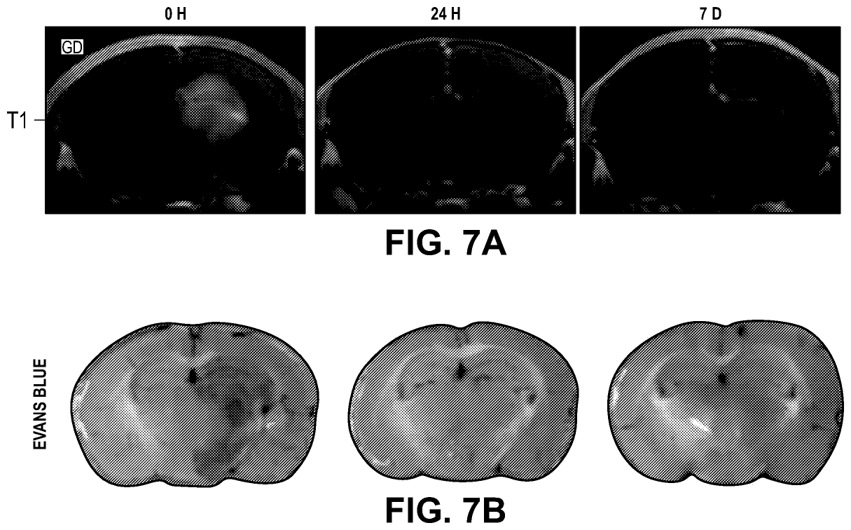

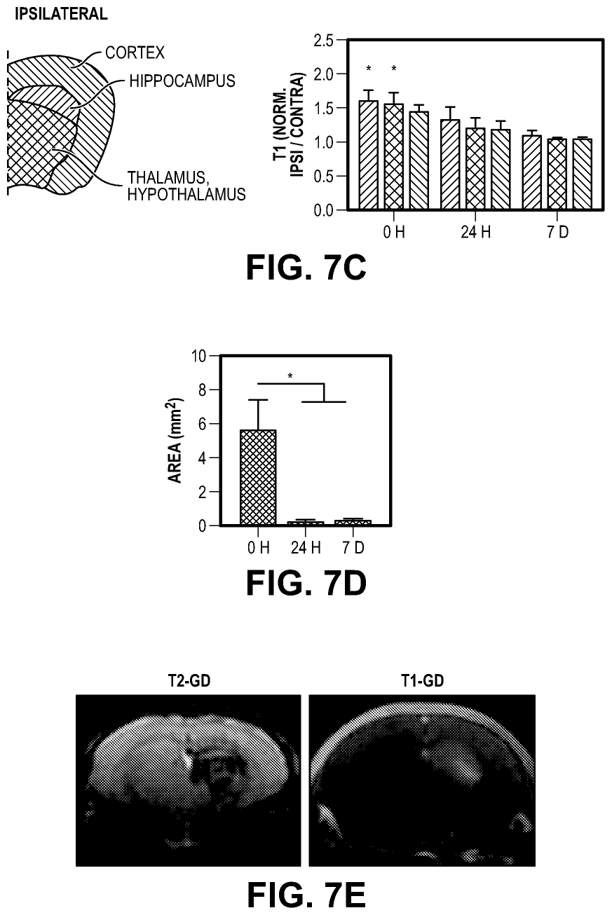

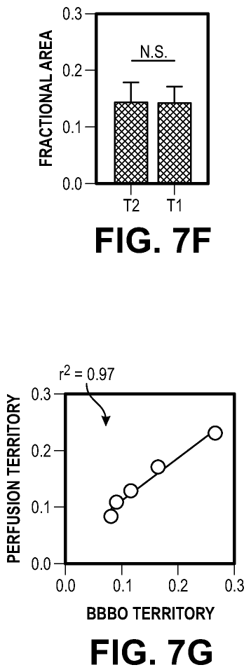

Utilizing dynamic T1-weighted MRI, we found that BBBO occurred during injection of 3 μM·minute melittin, indicating nearly instantaneous disruption of brain capillaries. Injection resulted in Gadolinium (Gd) T1 hyperintensities and Evans Blue leakage immediately following dosing, which recovered at 24 hours and 7 days (FIG. 7A, 7B). Following dosing there was a significant increase in T1 intensity in the ipsilateral hippocampus and thalamus/hypothalamus compared to the contralateral counterpart (p=0.021 and 0.033, respectively) (FIG. 7C). T1 hyperintensity in the cortex was much lower indicating limited BBBO (p=0.134). Follow-up T1 MRI showed marginal leakage of Gd at 24 hours (p=0.154) and no Evans Blue extravasation, indicating rapid recovery of bather function. On day 7, both Gd-enhanced MRI (p=0.169) and Evans Blue confirmed full recovery of the BBB after 3 μM·minute melittin exposure. These observations were not brain-region specific; no statistically significant changes in T1 intensity were found in any brain regions at 24 hours and 7 days (p>0.05). Additionally, using thresholding of T1 intensity we found that a 3 M·minute dose opened a brain territory of 5.70±1.73 mm² within the hippocampus, which reverses to ˜20-fold less in area at 24 hours and 7 days (p=0.015 and 0.035, respectively) (FIG. 7D).

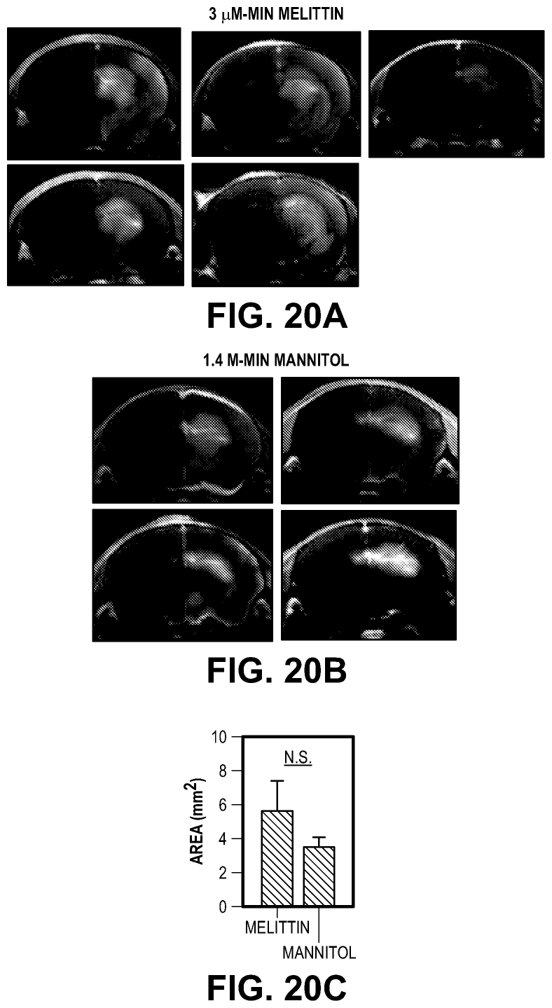

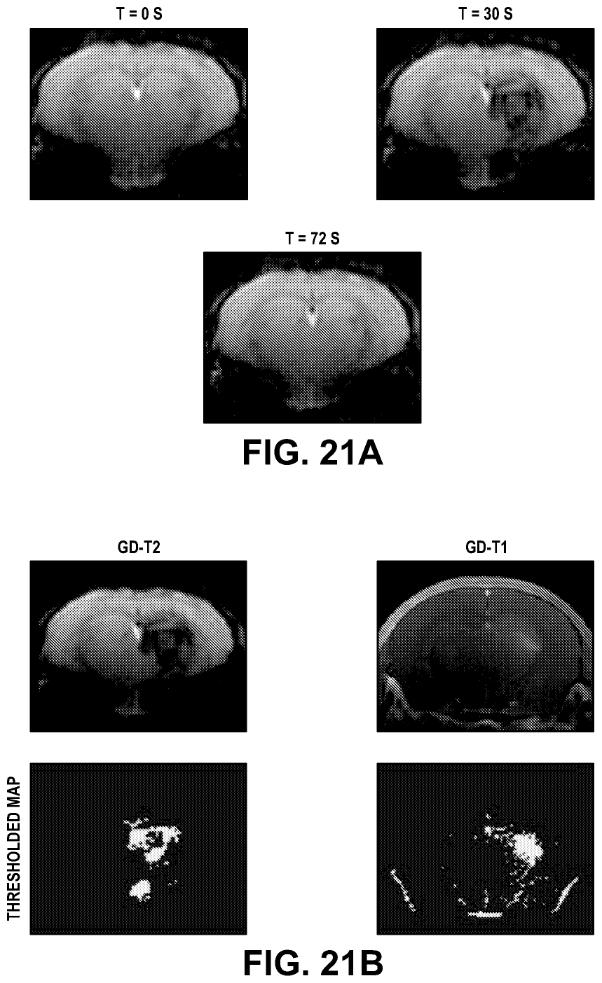

While 3 μM·minute melittin consistently induced BBBO, there was variability in the extent of opening, ranging from small regions limited to the hippocampal formation to large areas encompassing the majority of the ipsilateral hemisphere (FIG. 20A). However, this variability was not melittin-specific as hyperosmotic approaches utilizing injection of 1.4 M mannitol for 1 minute displayed similar variability patterns (FIG. 20B). Additionally, the extent of BBBO was similar between both approaches as measured by similar territories of opening (p=0.333) (FIG. 20C). Variability in opening stems from the highly integrated and collateralized cerebral circulation from the four major cerebral arteries and microregulation dynamically routing cerebral blood flow. This is a well-established phenomenon and a major driver of variability in clinical outcomes with intra-arterial drug delivery into the brain. See also D. S. Liebeskind, “Collateral circulation”, Stroke 34(9) (2003) 2279-84, which teaches that variability in opening territory is also observed using hyperosmotic BBBO and is likely due to differences in blood supply and collateral flow.

To address this challenge and improve the predictability of the territory of BBBO, we have developed a technique based on interventional MRI where injection of the BBB-opening agent is preceded by dynamic contrast-enhanced MRI during intra-arterial infusion of a contrast agent (FIG. 7D) as previously described. Intra-arterial infusion of saline supplemented with 50 mM gadolinium with dynamic T2-weighted MR imaging facilitates detection of a hypointense signal from a brain region selectively perfused by the microcatheter. The infusion rate can be manipulated to achieve effective perfusion of a desired brain territory, and at that point, melittin may be infused intra-arterially to the same brain region. With that approach we were able to predict BBBO territory as there is excellent agreement between contrast-perfusion territory and BBBO territory (FIGS. 7E, F and 21A-21B) (r²=0.97, p=0.002 significantly non-zero slope).

H. Safety of Melittin-Induced BBBO In Vivo

In 11 mice treated with 3 μM·minute melittin, all animals except one woke up from anesthesia without apparent neurological deficits (paralysis, seizure, or rotation). One mouse in this group died within 12 hours, possibly due to complications of surgery. In the remaining mice, there was no evidence of neurological deficits and the cohort dedicated for long-term survival (30 days) reached that endpoint without complications.

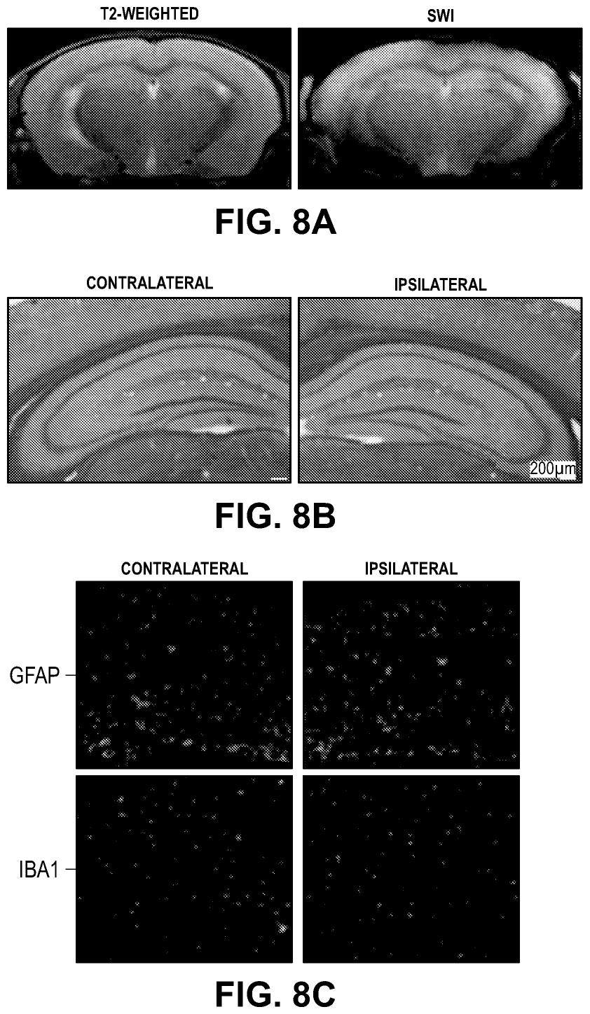

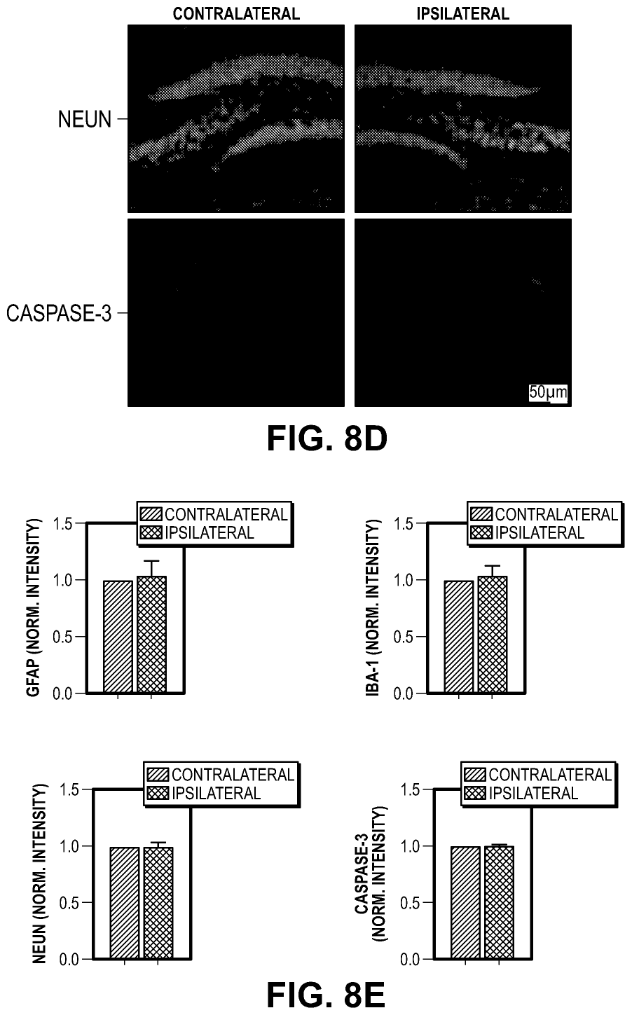

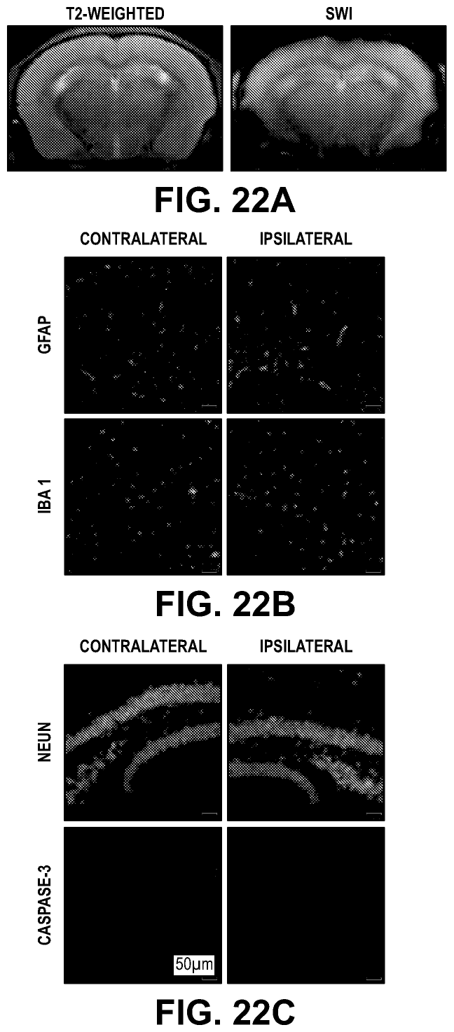

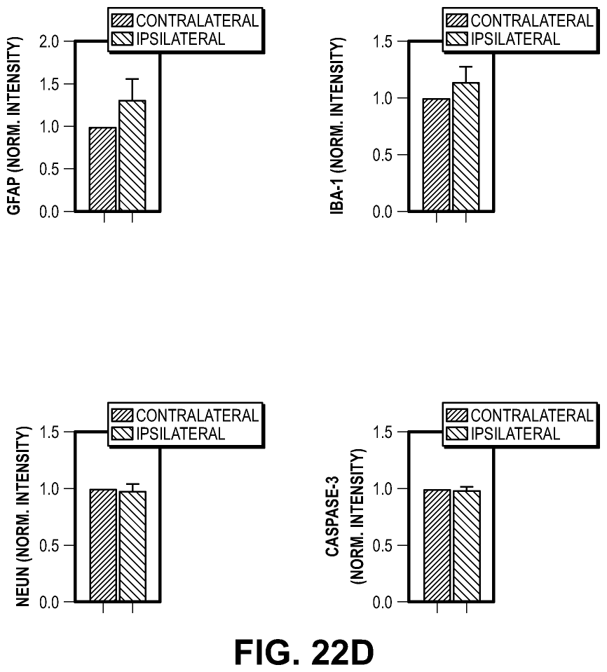

The effects of melittin-induced BBBO on edema and inflammation were studied for a 3 μM·minute dose at acute (24 hours) and chronic (7 days) time points. Follow-up MRI at 24 hours following a 3 μM·minute dose showed no evidence of edema or inflammation on T2 scans or micro-hemorrhages on susceptibility weighted imaging (SWI) scans (FIG. 22A). Similarly, no abnormalities were detected in MRI at 7 days (FIG. 8A), providing compelling evidence of neurological safety. Since the hippocampus was the region with the most consistent BBBO, we focused on that region for histopathological assessment. Staining for eriochrome cyanine revealed normal myelin at 7 days (FIG. 8B). The activation of astrocytes and microglia was assessed from immunohistochemistry images of glial fibrillary acidic protein (GFAP) and ionized calcium binding adaptor molecule 1 (Iba1) (FIG. 8C). Both sensitive markers of neuroinflammation showed no statistically significant differences in intensity between the ipsilateral and contralateral hemispheres 7 days after treatment (p=0.628 and 0.372, respectively) (FIG. 8E). Neuronal damage was assessed from immunohistochemistry images of the neuron-specific marker NeuN and the apoptotic marker caspase-3 (FIG. 8D). There was no statistically significant change in the intensity of both markers in the ipsilateral hemisphere 7 days after treatment (p=0.933 and 0.562, respectively) (FIG. 8E). We also evaluated neuroinflammation and neuronal damage at the acute stage, 24 hours after intra-arterial injection (FIG. 22B-D). Both T2 and SWI appeared normal, and GFAP, Iba1, NeuN, and caspase-3 intensities were not statistically different between hemispheres (p>0.05 for all cases).

I. Summary

We demonstrate that membrane-active peptides, such as melittin and variants thereof, induce reversible blood-brain barrier opening via enhanced paracellular permeability, e.g., in a tissue-engineered blood-brain barrier microvessel model. A scrambled melittin peptide sequence did not induce blood-brain barrier opening, demonstrating sequence specificity. Accordingly, membrane-active peptides of the disclosure can be engineered with customizable pharmacological properties via amino acid substitutions and the ability to incorporate unnatural amino acids with a variety of properties (e.g., charge, hydrophobicity, amphipathicity).

Further, when administered as an intra-arterial injection into the mouse cerebrovasculature, membrane-active peptides, such as melittin, support reversible blood-brain barrier opening without neurological damage. These results support that membrane-active peptides can be used to enhance brain penetration of diverse therapeutic agents.

More particularly, we have shown that membrane-active peptides, such as melittin, dramatically enhanced delivery of 500 kDa dextran across the blood-brain barrier in vitro (up to a 25-fold increase). This demonstrates the potential to deliver a wide range of therapeutic agents, including antibodies (MW ˜150 kDa), which display negligible penetration into the healthy brain. Further, combining membrane-active peptides with intra-arterial delivery as described herein minimizes systemic toxicity and dramatically improves brain accumulation by several orders of magnitude. This is in contrast to MR-guided focused ultrasound (FUS), which, as noted previously, confers risk for off-target effects and is highly inefficient as only a small fraction of therapeutics enter the brain (as low as 0.009% of injected dose) due to systemic drug administration.

Comparison of mapBBBO as described herein and hyperosmotic blood-brain bather opening in vivo showed that the dynamics were similar; however, mapBBBO occurred well below the solubility limit of e.g., melittin. Intra-arterial injection with 3 μM·min melittin (˜120-fold below solubility limit) resulted in robust opening of the hippocampus and thalamus/hypothalamus. This suggests that membrane-active peptide blood-brain barrier opening could be implemented without complete displacement of blood volume as required for mannitol which only induces opening close to its solubility limit.

The variability in opening territory following mapBBBO is also observed using hyperosmotic BBBO and is likely due to differences in blood supply and collateral flow. To address this variability, we utilized real-time imaging of the catheter perfusion territory as described herein to monitor mapBBBO, which indicates feasibility for clinical use. Previously, as described herein, we have shown with interventional dynamic susceptibility contrast-enhanced MRI that the local trans-catheter perfusion territory is indeed highly variable (U.S. Patent Publication No. 2017/0079581). In addition to visualizing trans-catheter perfusion, interventional MRI is useful for improving the precision and predictability of the blood-brain barrier opening territory (U.S. Patent Publication No. 2017/0079581).

Further, as previously reported, in mice, the LD50 dose for intraperitoneal administration of an exemplary membrane-active peptide, melittin, is 3.2 mg kg⁻¹. While the regional concentration of melittin in the brain was high after intra-arterial administration as demonstrated in the above examples, the systemic dose was ˜50-fold lower than the LD50 (0.64 mg kg⁻¹ for 3 μM·min melittin, assuming a mouse weight of 20 g). Additionally, assuming dilution in the bloodstream upon exiting the brain, melittin concentrations would be reduced ˜10-fold (0.33 μM for 3 μM·min melittin, assuming a 150 μL injection is diluted into 1.2 mL of whole blood). Thus, utilizing intra-arterial delivery of the present membrane-active peptides into the arteries can avoid systemic toxicity.

Although the majority of enhanced permeability into the brain occurs after the membrane-active peptide doses, e.g. a melittin dose (as observed at intermediate doses in tissue-engineered microvessels), blood-brain barrier disruption during perfusion indicates that membrane-active peptides, such as melittin (MW 2.7 kDa) can be transported into the surrounding tissue during the perfusion. Nevertheless, in our cytotoxicity assay we showed that HCNs maintained high viability (≥98%) for ˜12 minutes in 5 μM and 10 μM melittin, and ˜23 minutes for 0.75 μM melittin. For in vivo experiments we used a 1-minute dose and hence neuronal exposure to melittin in the vicinity of focal leaks is expected to be short. Therefore, membrane-active peptides, such as melittin, are unlikely to cause significant neurotoxicity, even at relatively high local doses. This conclusion was further supported by in vivo toxicity studies, finding limited neurotoxicity, demyelination or neuroinflammation after 7 days or neurological deficits for up to 30 days.

The safe dose range for intra-arterial injection was relatively narrow in mice since 1 μM·minute melittin did not result in blood-brain barrier opening and 5 μM·minute melittin resulted in neurological deficits. A narrow regime of neurological safety is also observed in mice for hyperosmotic blood-brain barrier opening, which requires high injection speeds to achieve complete blood displacement.

Given interspecies differences between mice and humans, we took an unconventional approach for development of a drug delivery system—utilization of a tissue-engineered model to motivate in vivo experiments and elucidate mechanisms. We maximized physiological relevance by using human-derived cells and minimized use of animal models. Our tissue-engineered microvessel model recapitulated features of the human blood-brain barrier, including physiological permeability, cylindrical geometry, cell-matrix interactions, and shear stress. This platform provides real-time resolution to assess the dynamics of transport (dextran molecule leakage) and endothelial cell behavior (contraction, proliferation, apoptosis) following melittin treatment. Animal models may not always predict clinical translation. Thus, human in vitro models provide an alternative approach for accelerating clinical translation. Our model mimics intra-arterial delivery of drugs injected into the cerebrovasculature, thus avoiding complexities of pharmacokinetics due to systemic delivery, which have been the focus of other human-on-a-chip in vitro systems.

Utilizing our tissue-engineered microvessels, we identified mechanistic features of membrane-active peptide blood-brain barrier opening: (1) increased paracellular (but not transcellular) permeability, (2) focal leaks associated with cell contraction, and (3) reversible junction disruption based on dose. Membrane-active peptides, such as melittin did not globally change the localization or expression of tight junctions (as observed using immunocytochemical detection of zona occludens-1). Instead, we observed that membrane-active peptides, such as melittin, induced local cell contraction. This matches previous studies which find that melittin causes calcium influx due to pore formation in cell membranes. Calcium flux into the endothelium is a regulator of cytoskeletal organization and barrier function and has been shown to induce disruption of tight junctions; other chemical blood-brain barrier opening agents are reported of function via a similar mechanism.

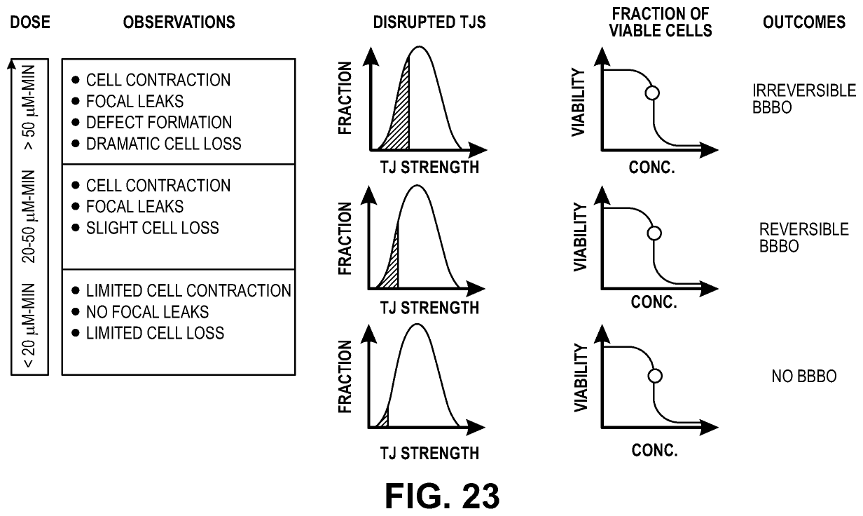

We propose, without being limited by theory, that cell contraction induces strain on surrounding cells that, if sufficiently large, transiently disrupts cell-cell junctions resulting in a focal leak (FIG. 23). At low doses (below 20 μM·minute in microvessels), the tensile forces on cell-cell junctions are insufficient to induce focal leak formation and the monolayer is able to maintain integrity. However, at intermediate doses (20 μM·minute-50 μM·minute) the tensile forces resulting from cell contraction were sufficiently large to induce local disruption of weak cell junctions resulting in the formation of focal leaks. The density of contraction and disruption events was sufficiently low that they are isolated, and recovery of normal barrier function occurred rapidly. At high doses (100 μM·minute) the density of contraction events was sufficiently high that clusters of disrupted cell-cell junctions can result in cell loss; however, this regime is not observed in vivo as hemorrhaging does not occur.

We identified an approximately 7-fold difference in melittin dose required for blood-brain barrier opening of human microvessels (20 μM·minute) compared to the mouse brain (3 μM·minute), which is likely due to species-to-species differences in the BBB, as other brain permeability enhancers appear to display a dose mismatch between rodents and humans. Nevertheless, the findings indicate that the present membrane-active peptides have clinical applications for delivery of therapeutics into the brain.

1. A composition or combination comprising:
at least one membrane-active peptide comprising an amino acid sequence represented by Formula (I):
X₁X₂X₃X₄X₅X₆Z₁X₇X₈Z₂Z₃X₉X₁₀X₁₁X₁₂X₁₃X₁₄Z₄X₁₅X₁₆U₁U₂U₃U₄U₅U₆-B₁ (I),
wherein X₁-X₁₆ are each independently selected from a natural or a non-natural hydrophobic amino acid residue;
wherein Z₁-Z₄ are each independently selected from a natural or a non-natural hydrophilic or hydrophobic amino acid residue, and wherein at least two of Z₁-Z₄ is independently selected from a natural or a non-natural hydrophilic amino acid residue;
wherein U₁-U₆ are each independently selected from a natural or a non-natural hydrophobic or hydrophilic amino acid residue, and wherein at least three of U₁-U₆ is a natural or a non-natural hydrophilic amino acid residue,
wherein B₁ is a terminus selected from COO— or CONH₂;
at least one therapeutic and/or diagnostic agent; and
wherein the composition or combination is optionally formulated for intraarterial injection.

2. The composition or combination of claim 1, wherein at least one of Z₁, X₁-X₁₆ and/or U₁-U₆ is proline or a non-natural analog thereof.

3. The composition or combination of claim 1, wherein at least one of Z₁, X₁, X₂, X₇, X₈, Z₂, Z₃, X₉, X₁₀, X₁₁, X₁₅ and/or X₁₆ is proline or a non-natural analog thereof and wherein X₃-X₆, X₁₂-X₁₄, Z₄ and U₁ to U₆ are not proline or a non-natural analog thereof.

4. The composition or combination of claim 1, wherein at least one of Z₁, X₇, X₈, Z₂, Z₃, X₉, X₁₀ and/or X₁₁ is proline or a non-natural analog thereof and wherein X₁-X₆, X₁₂-X₁₄, X₁₅, X₁₆, Z₄ and U₁ to U₆ are not proline or a non-natural analog thereof.

5. The composition or combination of claim 1, wherein Formula I does not contain proline or a non-natural analog thereof.

6. The composition or combination of claim 1,
wherein X₁, X₃ and X₉ are glycine, alanine or a non-natural analog thereof,
wherein X₂, X₄, X₅, X₆, X₈, X₁₀, X₁₂, X₁₃, X₁₄ X₁₆ are each independently selected from the group consisting of isoleucine, alanine, glycine, valine, leucine and a non-natural analog thereof;
wherein Z₁ is a natural or non-natural basic amino acid residue;
wherein X₇ is selected from the group consisting of valine, leucine, isoleucine, glycine, alanine and a non-natural analog thereof;
wherein Z₂ is selected from the group consisting of threonine, serine, alanine and a non-natural analog thereof;
wherein Z₃ and Z₄ are each independently selected from the group consisting of threonine, serine, alanine and a non-natural analog thereof;
wherein X₁₁ is proline or a non-natural analog thereof;
wherein X₁₅ is selected from the group consisting of tryptophan, phenylalanine, tyrosine, and a non-natural analog thereof;
wherein U₁, U₄ and U₅ are each independently selected from a natural or non-natural hydrophilic amino acid residue and
wherein U₂, U₃ and U₆ are each independently selected from a natural or a non-natural hydrophilic or hydrophobic amino acid residue.

7. The composition or combination of claim 6,
wherein the at least one membrane-active protein is represented by Formula II:
GIGAVLZ₁X₇LZ₂TGLPALISWIU₁U₂U₃U₄U₅U₆-B₁ (II),
wherein Z₁ is selected from the group consisting of lysine, arginine, histidine and a non-natural analog thereof;
wherein X₇ is selected from valine, leucine, isoleucine, glycine, alanine or a non-natural analog thereof;
wherein Z₂ is selected from threonine, alanine, serine or a non-natural analog thereof; and
wherein U₁-U₆ are each independently selected from the group consisting of lysine, arginine, histidine, alanine, threonine, serine, leucine, valine, isoleucine, glycine, glutamine and a non-natural analog thereof.

8. The composition or combination of claim 1,
wherein X₁, X₃ and X₉ are glycine or a non-natural analog thereof,
wherein X₂, X₄, X₅, X₆, X₈, X₁₀, X₁₂, X₁₃, X₁₄ X₁₆ are each independently selected from the group consisting of isoleucine, alanine, valine, leucine and a non-natural analog thereof;
wherein Z₁ is a natural or non-natural basic amino acid residue;
wherein X₇ is selected from the group consisting of valine, glycine and a non-natural analog thereof;
wherein Z₂ is selected from the group consisting of threonine, alanine and a non-natural analog thereof;
wherein Z₃ and Z₄ are each independently selected from the group consisting of threonine, serine and a non-natural analog thereof;
wherein X₁₁ is proline or a non-natural analog thereof;
wherein X₁₅ is selected from the group consisting of tryptophan, phenylalanine, tyrosine, and a non-natural analog thereof;
wherein U₁, U₄ and U₅ are each independently selected from a natural or non-natural hydrophilic amino acid residue and
wherein U₂, U₃ and U₆ are each independently selected from a natural or a non-natural hydrophilic or hydrophobic amino acid residue.

9. The composition or combination of claim 8,
wherein the at least one membrane-active protein is represented by Formula II:
GIGAVLZ₁X₇LZ₂TGLPALISWIU₁U₂U₃U₄U₅U₆-B₁ (II),
wherein Z₁ is selected from the group consisting of lysine, arginine and a non-natural analog thereof;
wherein X₇ is selected from valine, glycine or a non-natural analog thereof;
wherein Z₂ is selected from threonine, alanine or a non-natural analog thereof; and
wherein U₁-U₆ are each independently selected from the group consisting of lysine, arginine, alanine, leucine, glutamine and a non-natural analog thereof.

10. The composition or combination of claim 1, wherein the at least one active membrane peptide comprises SEQ ID NO: 1.

11. The composition or combination of claim 1, wherein the at least one active membrane peptide is selected from the group consisting of SEQ ID NOS: 2-11.

12. The composition or combination of claim 1, wherein an N-terminus of the membrane-active peptide is acetylated or conjugated to a fatty acid or a sterol.

13. The composition or combination of claim 1, wherein the at least one membrane-active peptide ranges from 26-100 amino acids in length.

14. The composition or combination of claim 1, wherein the at least one membrane-active peptide ranges from 26-50 amino acids in length.

15. The composition or combination of claim 1, wherein the at least one membrane-active peptide is 26 amino acids in length.

16. A method of opening a blood-brain barrier in a subject comprising:
administering to a subject the at least one membrane-active peptide according to claim 1.

17. The method of claim 16, wherein the opening of the blood-brain barrier is reversible.

18. The method of claim 16, further comprising adjusting an infusion rate of the at least one membrane-active peptide.

19. The method of claim 16, further comprising adjusting a length of time of perfusion of the at least one membrane-active peptide.

20. A method of delivering a therapeutic and/or a diagnostic agent to a central nervous system (CNS) of a subject in need thereof comprising:
administering to the subject the composition or combination according to claim 1.

21. The method of claim 20, wherein said administering further comprises:
administering the composition or combination comprising the at least one membrane-active peptide to an isolated region of a brain via a catheter, thereby opening a region of a blood-brain barrier;
administering a contrast agent to said isolated region via the catheter;
locating the regional opening in the blood-brain barrier, wherein said locating comprises non-invasive magnetic resonance imaging; and
administering a therapeutically effective amount of a therapeutic or diagnostic agent to the located regional opening in the blood-brain barrier.

22. The method of claim 21, wherein the catheter is an intraarterial catheter.

23. The method of claim 22, wherein the intraarterial catheter is located in an artery selected from a basilar artery or a carotid artery.

24. The method of claim 21, wherein the contrast agent is selected from the group consisting of gadolinium, feraheme, gadoterate, gadodiamide, gadobenate, gadopentetate, gadoteridol, gadoversetamide, gadoxetate, gadobutrol, gadofosveset and a combination thereof.

25. The method of claim 20, wherein the therapeutic or diagnostic agent is administered to the subject after administering the composition or combination comprising the at least one membrane-active peptide.

26. The method of claim 20, wherein the subject has a neurological disorder or a proliferative disorder.

27. The method of claim 26, wherein the proliferative disorder is cancer.

28. The composition or combination of claim 1, wherein the therapeutic agent is selected from the group consisting of an inorganic molecule, a peptide, a peptide mimetic, an antibody, a nucleic acid molecule and a combination thereof.

29. The method of claim 16, wherein the at least one membrane active peptide, composition or combination is administered by systemic intravenous administration.

30. The method of claim 16, wherein the at least one membrane active peptide, composition or combination is administered intra-arterially.
